(19) United States
(12) Patent Application Publication
(10) Pub. No.: US 2007/0248700 A1
(43) Pub. Date: Oct. 25, 2007

(54) Extractions and Methods Comprising Elder Species

(76) Inventors: Randall S. Alberte, Falmouth, ME (US); Robert T. Gow, Naples, FL (US); George W. Sypert, Naples, FL (US); Dan Li, Singapore (SG)

Correspondence Address: FOLEY HOAG, LLP;PATENT GROUP, WORLD TRADE CENTER WEST, 155 SEAPORT BLVD, BOSTON, MA, 02110, US

(21) Appl. No.: 11/687,897

(22) Filed: Mar. 19, 2007

Related U.S. Application Data
(60) Provisional application No. 60/783,453, filed on Mar. 17, 2006.
(60) Provisional application No. 60/846,412, filed on Sep. 22, 2006.
(60) Provisional application No. 60/873,473, filed on Dec. 7, 2006.

(51) Int. Cl. A61K 36/35; A61K 127/00; A61K 131/00; A61P 31/16; A61P 31/22; C07H 17/00; A61P 31/18; A61P 31/12; A61K 133/00

(52) U.S. Cl. 424/769; 536/8

(57) ABSTRACT

The present invention relates to extracts of elder species plant material prepared by supercritical CO₂ extractions methods, methods of treating viruses in a subject and methods of inhibiting viral infections in cells thereof.

### RELATED APPLICATIONS

This application claims the benefit of priority to U.S. Provisional Patent Applications Ser. Nos. 60/783,453, filed Mar. 17, 2006, 60/846,412, filed Sep. 22, 2006, and 60/873,473, filed Dec. 7, 2006, which are hereby incorporated by reference in their entirety.

### FIELD OF INVENTION

The present invention relates to extractions and methods thereof derived from Elder Sambuca species having uniquely elevated essential oil chemical constituents, phenolic acid chemical constituents, anthocyanidin or proanthocyanidin chemical constituents, or lectin-polysaccharide chemical constituents and extractions made by such methods, and methods for use of such extractions.

### BACKGROUND OF THE INVENTION

Elder, Sambuca nigra L., native to Europe, North Africa, and Western Asia, is a wild shrub. Elder further consists of over 20 Sambuca species, many of which have similar chemical constituents. Sambucus nigra L. is the species on which the majority of scientific research has been conducted. It is a deciduous tree growing to 10 m exhibiting cream white flowers and blue-black berries (elderberries). The flowers, leaves, and berries all contain chemical constituents of medical importance including essential oil compounds, phenolic acids, particularly the flavonoids and anthocyanidins, lectin protein compounds, and polysaccharide compounds.

The use of Sambuca species as medicines dates back to the fifth century BCE, included in the writings of Hippocrates, Dioscorides, and Pliny. Elder has a long history of traditional use among Native Americans and European herbalists. The traditional medical use and modern research activities have focused on the flower extracts.

The flowers are harvested in the spring and dried away from sunlight at below 40° C., to minimize loss of aroma. The berries are harvested in the fall when fully ripened. Most of the flowers and berries in commerce are imported from the Russian Federation, Poland, Hungary, Bulgaria, and Portugal. The berries are also used to add flavor and color, for wines, winter cordials, preserves, foods, and condiments. Both the flower and the berries have long histories as medicinal agents.

The chemical constituents of Sambucus nigra L. flowers and berries include the bioactive phenolic acids (flavonoids and anthocyanidins), proteins, polysaccharides, and vitamins (C, P, B1, B2, and B6). Although the information on the chemical constituents of Sambucas species flowers and berries are incomplete, the known chemical constituents are listed in Table 1. From a commercial and biological standpoint, the flavonoids and the anthocyanidins have been traditionally considered to be of greater importance than the other constituents.

TABLE 1
Chemical constituents of Sambucus nigra L. inflorescence and berries.

% mass weight

| Chemical Constituents | Flowers | Berries |
|---|---|---|
| Essential Oil | | |
| Volatile Oil | 0.04-0.31 | 0.01 |
| Linoleic acid | | |
| Linolenic acid | | |
| Palmitic acid | | |
| Triterpene | 1 | 1 |
| Alpha-amyrin | | |
| Beta-amyrin | | |
| Triterpene Acids | 0.85 | 0.9 |
| Ursolic acid | | |
| Oleanolic acid | | |
| Phenolic Acids | | |
| Flavonoid glycosides | 2-3 | 2-3 |
| Astragalin | | |
| Hyperoside | | |
| Isoquecitrin | | |
| Rutin | | |
| Aglycones | | |
| Quercetin | | |
| Kaempferol | | |
| Caffeic acid derivatives | 1-3 | 1-3 |
| Chlorogenic acid | | |
| Anthocyanidins | | |
| Cyanidin 3-sambubioside-5-glucoside | | |
| Cyanidin 3,5-diglucoside | | |
| Cyanidin 3-sambubioside | | |
| Cinanidin 3-glucoside | | |
| Tannins | | |
| Alkanes | | |
| Mucilage | | |
| Pectin | | |
| Protein (plastocynin) | | |
| Carbohydrates | | |
| Monosaccharides | | |
| Polysaccharides | | |
| Minerals | 8-9 | 3-9 |

The medicinal properties of elder species results in the presence of its pharmacologically active chemical constituents. As a general rule for chemical contents, the strongly colored berries contain high levels of anthocyanidins as pigments, as well as flavonol glycosides and aglycones (Espin J C et al. J Agric Food Chem 48:1588-1592, 2000; Kahkonen M P et al. J Agric Food Chem 49:4076-4082, 2001).

Anthocynidins are glycosylated-polyhydroxy and -polymethoxy derivatives of 2-phenylbenzopyrylium salts (Brouillard KaHSH. Chemical Structure of Anthocyanins. Academic Press, New York, 1982). Elderberries are one of the richest sources of these pigments, having contents of 0.2-1%, which is far higher than that found in grapes (Bronnum-Hansen K et al. J Food Technology 20:703-711, 1985). Elderberry contains several different anthocyanins of which cyanidin-3-sambubioside (compound 1) and cyanidin-3-glucoside (compound 2) are quantitatively the most important, accounting for more than 85% of the anthocyanidin content, whereas cyandin-3-sambucioside-5-glucoside (compound 3) and cyanidin-3,5-diglucoside (compound 4) are only present in minor amounts (Bronnum-Hansen K et al. J Chromatography 262:393-396, 1983; Drdak M & Daucik P. Acta Aliment 19:3-7, 1990). Anthocyanidins exhibit a range of biological activities. One of the best known attributes is the antioxidant activity, especially of the cyanidin derivatives (Drdak M & Daucik P. Acta Aliment 19:3-7, 1990; Tsuda T et al. J Agric Food Chem 42:2407-2410, 1994).

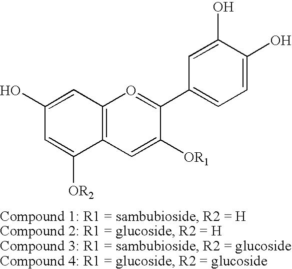

Testing different classes of bilberry phenolic acid compounds for their ability to inhibit colon cancer cell growth in vitro, it was found that the anthocyanidins are potent phenolics (Kamei H et al. Cancer Invest 13:590-594, 1995). Cyanidin in particular was very effective in inhibiting cell growth at a concentration as low as 2 μg/ml, which is only 1/10 of the concentration required for the potent anti-carcinogen genistein. Anti-cancer activity has also been noted for anthocyanins from blueberry (Smith M A L et al. J Food Sci 65:352-356, 2000).

Rutin and isoquercitrin are the main flavonol glycosides in elder species plant material (Pietta P & Bruno A. J Chromatography 593:165-170, 1992). These compounds have the capacity for acting as a potent radical scavenger (Shahidi F & Wannasundra P K. Crit Rev Food Sci Nutr 32:67-103, 1992; Rice-Evans C A et al. Free Radical Biol & Med 20:933-956, 1996), inhibiting a variety of enzymes (Formica J V & Regelson W. Food and Chem Toxic 33:1061-1080, 1995), and have an anti-hemorrhagic activity by tightening blood vessels (Dawidowicz A J et al. J Liquid Chromotog & Related Technologies 26:2381-2397, 2003). In studies using accelerated solvent extraction of Sambucus nigra flower, berry and leaf, rutin was found to be the major flavonoid. Flower had the highest amount of rutin and isoquercitrin in concentration of 2-3% and 0.1%. Elderberries and leaves have similar amount of rutin at concentration of about 0.2%. The results are shown in Table 2.

TABLE 2

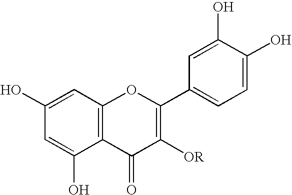

Extraction yield by 80% methanol of rutin and isoquercitrin from different
parts of S. nigra L.

| | Rutin (%) | Isoquercitrin (%) |
|---|---|---|
| Flower | 2-2.88 | 0.114 |
| Leaves | 0.14-0.2 | 0.003-0.005 |
| Berries | 0.16-0.19 | 0.02-0.03 |

Rutin: R = rutinoside
Isoquercitrin: R = glucoside

Elder species plant material possesses a pleasant strong smell due to its volatile constituents. Several alkanes have been identified in the elder leaves with heptacosane, nonacosane and hentriacontanes being quantitatively the most important ones. The essential oil of elder flowers is high in fatty acids (66%) and n-alkanes (7.2%). 79 compounds have been identified from steam distillation of elder flower essential oil (Toulemonde B et al. J Agric Food Chem 31:365-370, 1983). The major constituents of the essential oil were trans-3,7-dimethyl-1,3,7-octatrien-3-ol (13%), palmitic acid (11.3%), linalool (3.7%), cis-hexenol (2.5%) and cis- and trans-rose oxides (3.4% and 1.7% respectively).

The principal commercial elderberry extract contains an anthocyanidin concentration of 0.5% (Espin J C et al. J Agric Food Chem 48:1588-1592, 2000). The predominant anthocyanidins were cyanidin-3-monoglycoside (97%) and cyanidin-3,5-diglycoside (3%). The concentrate was also characterized by the presence a caffeic acid derivative (0.011%) and rutin (0.055).

The triterpenes and flavonoids have long been thought to be principal chemical constituents responsible for the biological activity of Sambucas species (Blumenthal M et al. Herbal Medicine: Expanded Commission E Monographs, Integrative Medicine Communications, Newton, Mass., 2000, pp. 103-105). However, the four major anthocyanidins appear to play a significant role in the anti-flu activity of Sambucas species. These anthocyanidins are incorporated into the plasma membrane and cytosol of endothelial cells following a 4-hour exposure to a Sambucas extract (Youdin K A et al. Free Radic Biol Med 29:51-60, 2000). Both human and animal endothelial cell enrichment with Sambucas species anthocyanidins appear to confer protective effects against oxidative stressors. Moreover, an extract of Sambucas species berries has been shown to have oxygen radical absorption capacity (Roy S et al. Free Radical Res 36:1023-1031, 2002). Sambucas species lectin and ribosomes-inactivating proteins also demonstrate anti-viral activity (Vanderbussche F et al. Eur J Biochem 27:1508-1515, 2004; de Benito F M et al. FEBS Lett 428:75-79, 1998; Fujimura Y et al. Virchows Arch 444:36-42, 2003). A standardized extract of S. nigra berries (Sambucol®. Razei Bar, Jerusalem) (4 g adult dose), contains 38% black elderberry extract with anthocyanidins combined with Echinacea angustifolica (rhizome) extract, Echinacea purpura (stem, leaf, & flower) extract, Vitamin C (100 mg) and zinc (10 mg) has been shown to exhibit the following properties: inhibition of hemagglutination produced by influenza viruses in humans (Zakay-Rones Z et al. J Alternative & Complementary Medicine 1:361-369, 1995); inhibition of viral replication in humans and in vitro (Zakay-Rones Z et al. J Alternative & Complementary Medicine 1:361-369, 1995); increased production of inflammatory and anti-inflammatory cytokines in humans (Barak V et al. Isr Med Assoc J 4 (suppl 11): 919-922, 2002); reduced hemagglutination and inhibition of replications of type A and type B human influenza viruses in vitro (Zakay-Rones Z et al. J Alternative & Complementary Medicine 1:361-369, 1995); reduction of infectivity of HIV in vitro (Zakay-Rones Z et al. J International Med Res 32:132-140, 2004); inhibition of HSV-1 strains replication in vitro (Zakay-Rones Z et al. J International Med Res 32:132-140, 2004); reduction of colitis in rat model (Bobek P et al. Biologia Bratislavia 56:643-648, 2002); reduction in influenza symptoms in chimpanzees (Gray A M et al. J Nutr 30:15-20, 2000); and a randomized clinical trial demonstrated reduction in influenza A and B symptoms in humans (Zakay-Rones Z et al. J International Med Res 32:132-140, 2004). Additional findings with other extraction compositions derived from S. nigra include: enhancement of lysosomal enzymes, reduction of production of lipoxygenation products, and reduction of myeloperoxidase activity in vitro (Bobek P et al. Biologia Bratislavia 56:643-648, 2002); protection against oxidative stress in vitro (Brouillard KaHSH. Chemical Structure of Anthocyanins. Academic Press, New York, 1982); increases in oxygen radical absorbing capacity in vitro (Bronnum-Hansen K et al. J Chromatography 262:393-396, 1983) and insulin-like and insulin-releasing actions in vivo (Gray A M et al. J Nutr 30:15-20, 2000).

To briefly summarize the therapeutic value of S. nigra's chemical constituents, scientific research and clinical studies have demonstrated the following therapeutic effects of the various chemical compounds, chemical groups, or extract compositions of Sambuca species which include: anti-viral, anti-common cold, anti-influenza, anti-HIV, anti-HSV (triterpenes, anthocyanidins, lectin proteins, polysaccharides, crude extracts); anti-oxidant and oxygen free radical scavenging (flavonoids, anthocyanidins, crude extract); anti-inflammatory activity (crude extract); anti-diabetes activity (polysaccharides, water soluble extract); regulation of bowel activity and moderation of diarrhea (extract); and reduction of agitation and restlessness (extract). In addition, S. nigra elder flower or elderberry extract compositions are generally considered safe with no known contraindications.

### SUMMARY OF THE INVENTION

In one aspect, the present invention relates to an elder species extract comprising a fraction having a Direct Analysis in Real Time (DART) mass spectrometry chromatogram of any of FIGS. 36 to 70. In a further embodiment, the fraction has a DART mass spectrometry chromatogram of any of FIGS. 46 to 50. In a further embodiment, the fraction has a DART mass spectrometry chromatogram of FIG. 48.

In one aspect, the present invention relates to an elder species extract comprising a fraction having an IC₅₀ of 150 to 1500 μg/mL as measured in a H1N1 influenza virus. In a further embodiment, the fraction has an IC₅₀ of 150 to 750 μg/mL. In a further embodiment, the fraction has an IC₅₀ of 150 to 300 μg/mL. In a further embodiment, the fraction has an IC₅₀ of at least 195 μg/mL.

In a further embodiment the present invention relates to an elder species extract of the present invention, wherein the fraction comprises an anthocyanin; flavonoid; C16 or C18 saturated or unsaturated fatty acid, alcohol, or ester; and/or a polysaccharide. In a further embodiment, the anthocyanin is selected from the group consisting of cyanidin-3-glucoside and cyanidin-3-sambucioside. In a further embodiment, the amount of anthocyanins is greater than 10, 20, 30, 40 or 50% by weight. In a further embodiment, the flavonoid is rutin. In a further embodiment, the C16 or C18 saturated or unsaturated fatty acid, alcohol, or ester is selected from the group consisting of hexadecanol, hexadecanoic acid, hexadecanoic acid methyl ester, hexadecanoic acid ethyl ester, hexadecanoic acid butyl ester, octadecanoic acid, octadecanoic acid ethyl ester, octadecanoic acid butyl ester, 9-octadecen-1-ol, 9,12-octadecanienoic acid, and combinations thereof. In a further embodiment, the amount of the C16 or C18 saturated or unsaturated fatty acid, alcohol, or ester is 2, 4, 6, 8, or 10% by weight. In a further embodiment, the polsaccharide is selected from the group consisting of dextran, glucose, arabinose, galactose, rhamnose, xylose, uronic acid, and combinations thereof. In a further embodiment, the amount of polysaccharide is 10, 15, 20, 25, 30, 35, or 40% by weight.

In a further embodiment, the present invention relates to an elder extract of the present invention wherein the fraction comprises an anthocyanin; C16 or C18 saturated or unsaturated fatty acid, alcohol, or ester; and a polysaccharide. In a further embodiment, the anthocyanin is selected from the group consisting of cyanidin-3-glucoside and cyanidin-3-sambucioside. In a further embodiment, the amount of anthocyanin is greater than 10, 20, 30, 40 or 50% by weight. In a further embodiment, the C16 or C18 saturated or unsaturated fatty acid, alcohol, or ester is selected from the group consisting of hexadecanol, hexadecanoic acid, hexadecanoic acid methyl ester, hexadecanoic acid ethyl ester, hexadecanoic acid butyl ester, octadecanoic acid, octadecanoic acid ethyl ester, octadecanoic acid butyl ester, 9-octadecen-1-ol, 9,12-octadecanienoic acid, and combinations thereof. In a further embodiment, the amount of the C16 or C18 saturated or unsaturated fatty acid, alcohol, or ester is 2, 4, 6, 8, or 10% by weight. In a further embodiment, the polysaccharide is selected from the group consisting of dextran, glucose, arabinose, galactose, rhamnose, xylose, uronic acid, and combinations thereof. In a further embodiment, the amount of polysaccharide is 10, 15, 20, 25, 30, 35, or 40% by weight.

In another aspect, the present invention relates to a food or medicament comprising the elder species extract of the present invention.

In another aspect, the present invention relates to a method of treating a subject for a viral infection comprising administering to the subject in need thereof an effective amount of the elder species extract of the present invention. In a further embodiment, the viral infection is caused by an envelope virus. In a further embodiment, the envelope virus is a flavie virus. In a further embodiment, the viral infection is caused by a non-envelope virus. In a further embodiment, the viral infection is caused by aninfluenza viruses, human flu viruses A and B, avian flu viruses, H1N1, H5N1, human immunodeficiency virus (HIV), SARs, herpes simplex viruses (HSV), flaviviruses, dengue, yellow fever, West Nile, and encephalitis viruses. In a further embodiment, the viral infection is caused by the Norwalk virus, hepatitis A, polio, andoviruses or a rhinoviruses. In a further embodiment, the subject is a primate, bovine, aviary, ovine, equine, porcine, rodent, feline, or canine. In a further embodiment, the subject is a human.

In another embodiment, the present invention relates to a method of inhibiting viral infection of cells comprising contacting the cells with the elder species extract of present invention. In a further embodiment, the viral infection is an envelope virus infection. In a further embodiment, the envelope virus infection is a flavie virus infection. In a further embodiment, the viral infection is a non-envelope virus infection. In a further embodiment, the viral infection is an influenza viruses, human flu viruses A and B, avian flu viruses, H1N1, H5N1, human immunodeficiency virus (HIV), SARs, herpes simplex viruses (HSV), flaviviruses, dengue, yellow fever, West Nile, and encephalitis viruses infection. In a further embodiment, the viral infection is a Norwalk virus, hepatitis A, polio, andoviruses or rhinoviruses infection.

In another aspect, the present invention relates to a method of preparing an elder species extract having at least one predetermined characteristic comprising: sequentially extracting an elder species plant material to yield an essential oil fraction, a polyphenolic fraction and a polysaccharide fraction by a) extracting an elder species plant material by supercritical carbon dioxide extraction to yield the essential oil fraction and a first residue; b) extracting either an elder species plant material or the first residue from step a) with water at about 40° C. to about 70° C. or a hydro-alcoholic extraction to yield the polyphenolic fraction and a second residue; and c) extracting the second residue from step b) by water at about 70° C. to about 90° C. extraction to yield the polysaccharide fraction. In another embodiment the extraction process can be carried out with any species rich in anthocyanidins and/or proanthocyanidins such as, for example, black currant berries, red currant berries, gooseberries, bilberries, blackberries, blueberries, cherries, cranberries, hawthorn berries, loganberries, raspberries, chokeberries, apples, pomegranates, quince, and plums.

In a further embodiment, obtaining the essential oil fraction comprises: 1) loading in an extraction vessel ground elder species plant material; 2) adding carbon dioxide under supercritical conditions; 3) contacting the elder species plant material and the carbon dioxide for a time; and 4) collecting an essential oil fraction in a collection vessel.

In a further embodiment, methods of the present invention further comprise the step of altering the essential oil chemical constituent compound ratios by fractionating the essential oil extraction with a supercritical carbon dioxide fractional separation system.

In a further embodiment, the polyphenolic fraction is obtained by 1) contacting ground elder species plant material or the residue from step a) with water at about 40° C. to about 70° C. or a hydro-alcoholic solution for a time sufficient to extract polyphenolic chemical constituents; 2) passing the hydro-alcoholic solution of extracted polyphenolic chemical constituents from step a) through an affinity adsorbent resin column wherein the polyphenolic acids including the anthocyanidins, are adsorbed; and 3) eluting the purified polyphenolic chemical constituent fraction(s) from the affinity adsorbent resin.

In a further embodiment, the method of obtaining the polysaccharide fraction comprises: 1) contacting the second residue from step b) with water at about 70° C. to about 90° C. for a time sufficient to extract polysaccharides; and 2) precipitating the polysaccharides from the water solution by ethanol precipitation.

In another aspect, the present invention relates to an elder species extract prepared by any of the methods of the present invention.

In another aspect, the present invention relates to an elder species extract comprising pyrogallol, methyl cinnamic acid at 15 to 25% by weight of the pyrogallol, cinnamide at 1 to 4% by weight of the pyrogallol, 2-methoxyphenol at 5 to 10% by weight of the pyrogallol, benzaldehyde at 1 to 2% by weight of the pyrogallol, cinnamaldehyde at 5 to 10% by weight of the pyrogallol, and cinnamyl acetate at 5 to 15% by weight of the pyrogallol.

In another aspect, the present invention relates to an elder species extract comprising rutin, ferulic acid at 20 to 30% by weight of the rutin, cinnamic acid at 25 to 35% by weight of the rutin, shikimic acid at 15 to 25% by weight of the rutin, and phenyllacetic acid at 55 to 65% by weight of the rutin.

In another aspect, the present invention relates to an elder species extract comprising rutin, taxifolin at 1 to 10% by weight of the rutin, ferulic acid at 1 to 5% by weight of the rutin, cinnamic acid at 1 to 5% by weight of the rutin, shikimic acid at 0.5 to 5% by weight of the rutin, phenyllacetic acid at 1 to 5% by weight of the rutin, cyanidin at 5 to 15% by weight of the rutin, and petunidin at 15 to 25% by weight of the rutin.

In another aspect, the present invention relates to an elder species extract comprising rutin, cyanidin at 30 to 40% by weight of the rutin, petunidin at 75 to 85% by weight of the rutin, vanillic acid at 5 to 10% by weight of the rutin, ferulic acid at 1 to 5% by weight of the rutin, and cinnamic acid at 1 to 10% by weight of the rutin.

In another aspect, the present invention relates to an elder species extract comprising p-coumaric acid/phenylpyruvic acid, rutin at 65 to 75% by weight of the p-coumaric acid/phenylpyruvic acid, vanillic acid at 65 to 75% by weight of the p-coumaric acid/phenylpyruvic acid, ferulic acid at 35 to 45% by weight of the p-coumaric acid/phenylpyruvic acid, cinnamic acid at 65 to 75% by weight of the p-coumaric acid/phenylpyruvic acid, and shikimic acid at 45 to 55% by weight of the p-coumaric acid/phenylpyruvic acid.

In another aspect, the present invention relates to an elder species extract comprising rutin, hesperidin at 20 to 30% by weight of the rutin, vanillic acid at 70 to 80% by weight of the rutin, and cinnamic acid at 40 to 50% by weight of the rutin.

In another aspect, the present invention relates to an elder species extract comprising petunidin, rutin at 85 to 95% by weight of the petunidin, vanillic acid at 55 to 65% by weight of the petunidin, and cinnamic acid at 30 to 40% by weight of the petunidin.

In another aspect, the present invention relates to an elder species extract comprising rutin, cyanidin at 5 to 15% by weight of the rutin, taxifolin at 1 to 10% by weight of the rutin, caffeic acid at 5 to 15% by weight of the rutin, ferulic acid at 1 to 10% by weight of the rutin, shikimic acid at 1 to 10% by weight of the rutin, petunidin at 25 to 35% by weight of the rutin, and eriodictyol or fustin at 1 to 5% by weight of the rutin.

In another aspect, the present invention relates to an elder species extract comprising rutin, cyanidin at 10 to 20% by weight of the rutin, eriodictyol or fustin at 1 to 5% by weight of the rutin, naringenin at 10 to 20% by weight of the rutin, and taxifolin at 1 to 10% by weight of the rutin.

These embodiments of the present invention, other embodiments, and their features and characteristics, will be apparent from the description, drawings and claims that follow.

### BRIEF DESCRIPTION OF DRAWINGS

FIG. 1 depicts an exemplary schematic diagram of elder species extraction processes in accordance with the present invention.

FIG. 2 depicts an exemplary schematic diagram of elder species extraction processes in accordance with the present invention.

FIG. 3 depicts an exemplary schematic diagram of elder species extraction processes in accordance with the present invention.

FIG. 4 depicts an exemplary schematic diagram of elder species extraction processes in accordance with the present invention.

FIG. 5 depicts viral entry assay system using human type A H1N1. MDCK cells were incubated with virus only (top left; 10-4 Flu A), no virus (bottom left; PBS), virus mixed with an anti-influenza virus antibody at a 1:1,000 concentration (top right; 1:1000 Ab) and a 1:500 concentration (bottom right; 1:500 Ab). Each experiment was done in triplicate. Each brownish red spot indicates one virus infection event. Virus inhibition or reduction in the number of colored spots is detected in the antibody controls.

FIG. 6 depicts an example of an inhibition assay using elderberry B anthocyanin fraction ADS5 desorption F4 and human influenza type A H1N1 virus. Serial dilutions (undiluted to 1:32 dilutions) of the elderberry B anthocynin fraction ADS5 desorption F4 fraction were pre-incubated with virus prior to incubating with MDCK cells. Each experimental was done in triplicate. Spots correspond to one virus infection event. Virus inhibition is indicated by a reduction in the number of spots.

FIG. 7 depicts an inhibition assay using elderberry B anthocynin fraction ADS5 desorption F4 and human influenza type A H1N1 virus. Serial dilutions (undiluted to 1:32 dilutions) of the elderberry B anthocynin fraction ADS5 desorption F4 fraction were pre-incubated with virus prior to incubating with MDCK cells. Each experimental was done in triplicate. Brownish red spots correspond to one virus infection event. Virus inhibition is indicated by a reduction in the number of colored spots.

FIG. 8 depicts an inhibition assay using elderberry B anthocynin fraction ADS5 desorption F4 and human influenza type A H₅N₁ virus. Serial dilutions (undiluted to 1:32 dilutions) of the elderberry B anthocynin fraction ADS5 desorption F4 fraction were pre-incubated with virus prior to incubating with MDCK cells. Each experimental was done in triplicate. Brownish red spots correspond to one virus infection event. Virus inhibition is indicated by a reduction in the number of colored spots.

FIG. 9 depicts the inhibition assay for chimeric HIV-1 SG3 (genome) subtype C (envelope). +, is the positive infection control; F4, is the elderberry extract fraction F4; and T is titration of virus used in the assay.

FIG. 10 depicts MTT viability assay for elderberry B anthocynin fractions ADS5 desorption F2 fraction in 293 T cells.

FIG. 11 depicts MTT viability assay for elderberry B anthocynin fractions ADS5 desorption F2 fraction in MDCK cells.

FIG. 12 depicts MTT viability assay for elderberry B anthocynin fractions ADS5 desorption F4 fraction in 293 T cells after 24 hours.

FIG. 13 depicts MTT viability assay for elderberry B anthocynin fractions ADS5 desorption F4 fraction in 293 T cells after 44 hours.

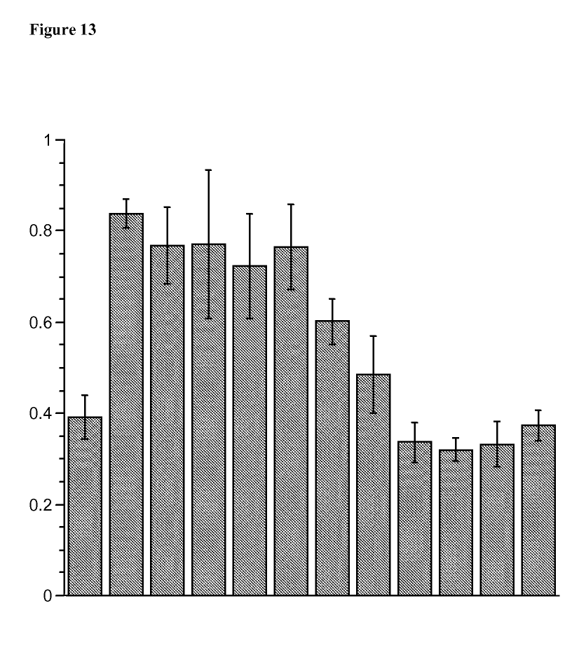

FIG. 14 depicts the infectivity inhibition dose response curve and 50% inhibitory concentration for elderberry B anthocynin fraction ADS5 desorption F2 fraction.

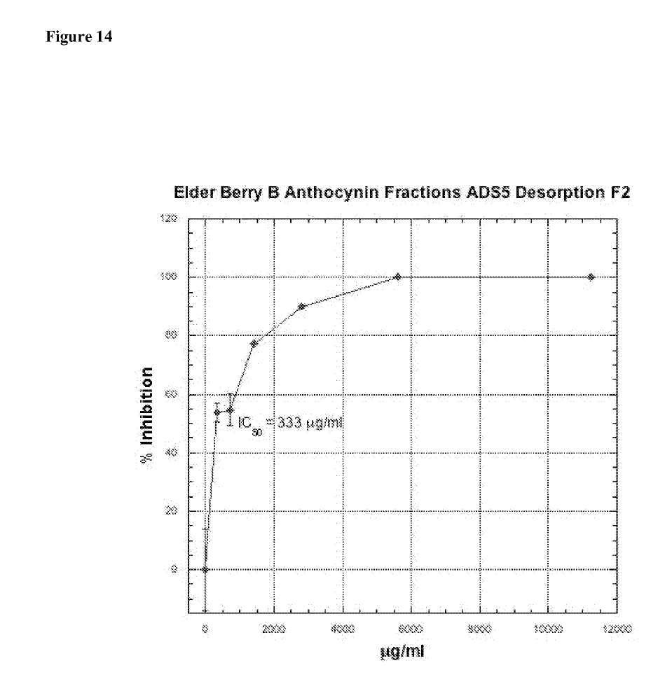

FIG. 15 depicts the infectivity inhibition dose response curve and 50% inhibitory concentration for elderberry B anthocynin fraction ADS5 desorption F3 fraction.

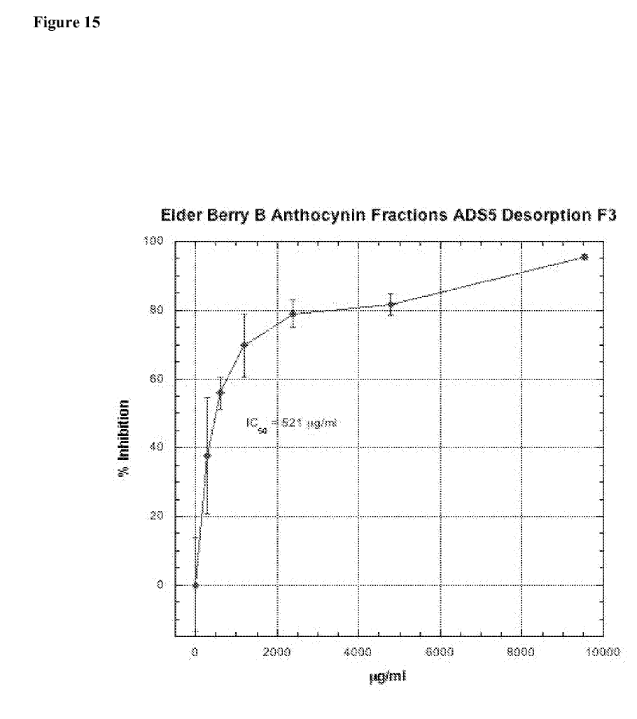

FIG. 16 depicts the infectivity inhibition dose response curve and 50% inhibitory concentration for elderberry B anthocynin fraction ADS5 desorption F4 fraction.

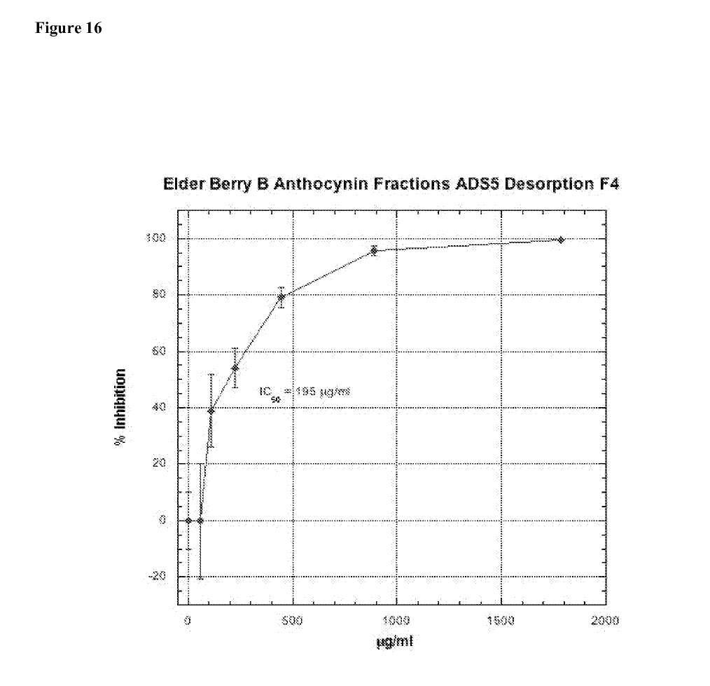

FIG. 17 depicts the infectivity inhibition dose response curve and 50% inhibitory concentration for elder flower XAD 7HP desorption F2 fraction.

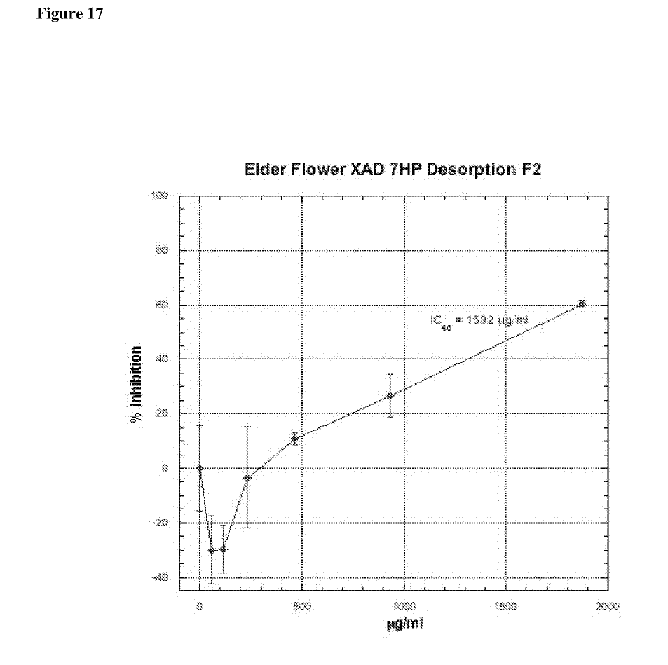

FIG. 18 depicts the infectivity inhibition dose response curve and 50% inhibitory concentration for elder flower XAD 7HP desorption F3 fraction.

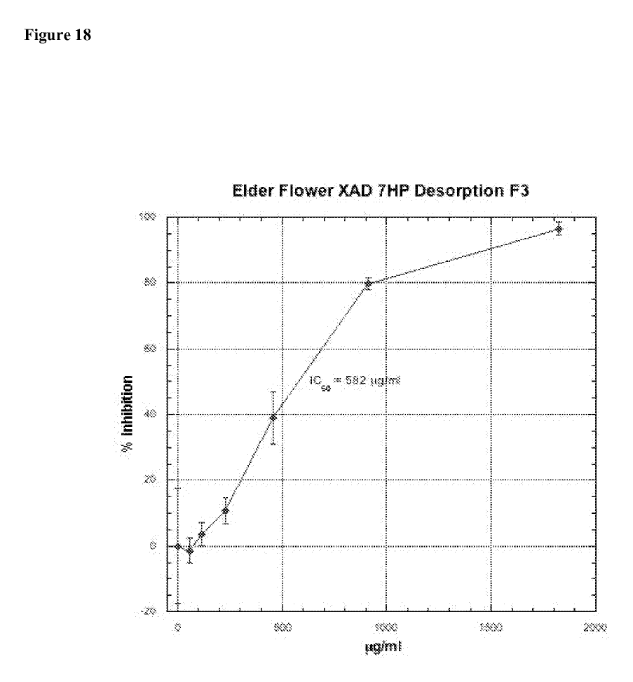

FIG. 19 depicts the infectivity inhibition dose response curve and 50% inhibitory concentration for unbuffered elderberry B anthocynin fraction ADS5 desorption F3 fraction using H1N1 virus.

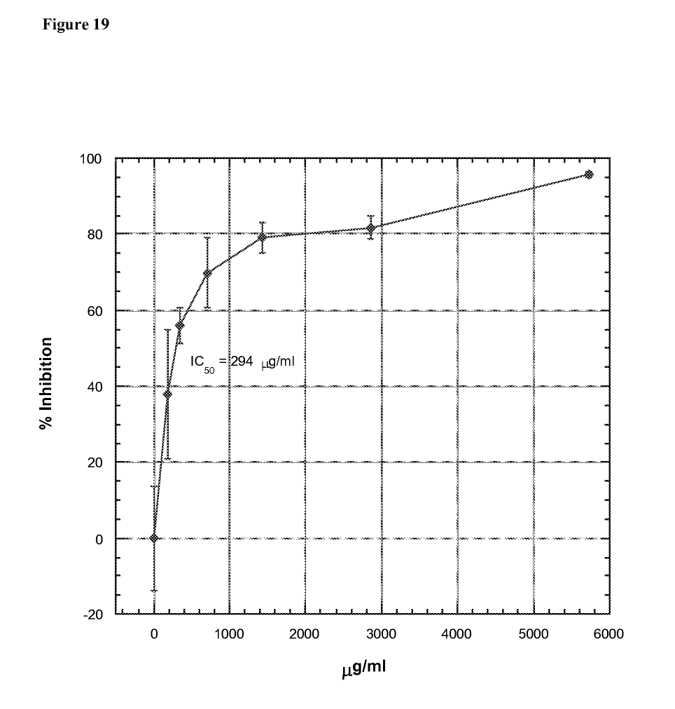

FIG. 20 depicts the infectivity inhibition dose response curve and 50% inhibitory concentration for unbuffered elderberry B anthocynin fraction ADS5 desorption F3 fraction using H1N1 virus.

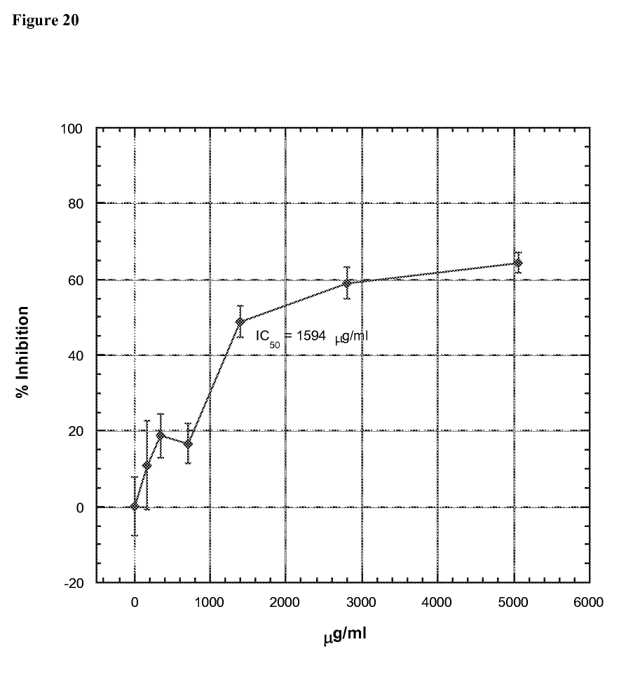

FIG. 21 depicts the infectivity inhibition dose response curve and 50% inhibitory concentration for unbuffered elderberry B anthocynin fraction ADS5 desorption F2 fraction using H1N1 virus.

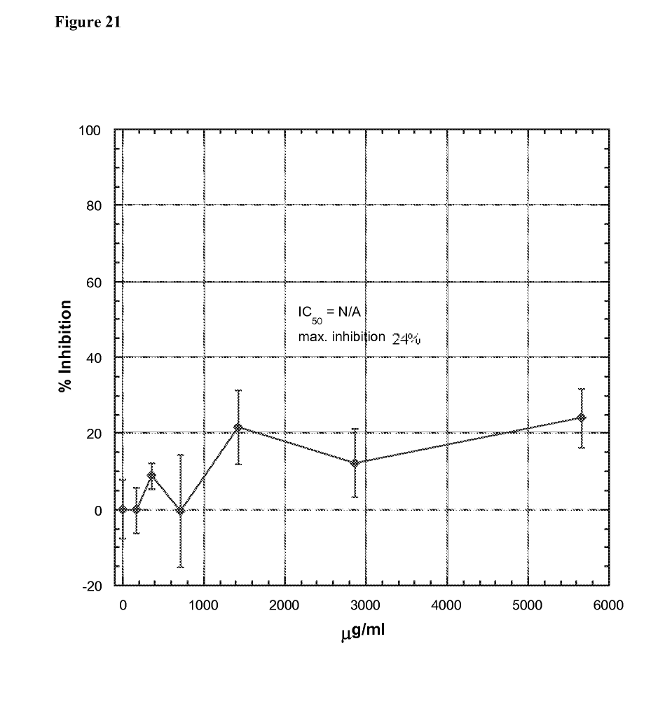

FIG. 22 depicts the infectivity inhibition dose response curve and 50% inhibitory concentration for unbuffered elderberry B anthocynin fraction ADS5 desorption F4 fraction using H1N1 virus.

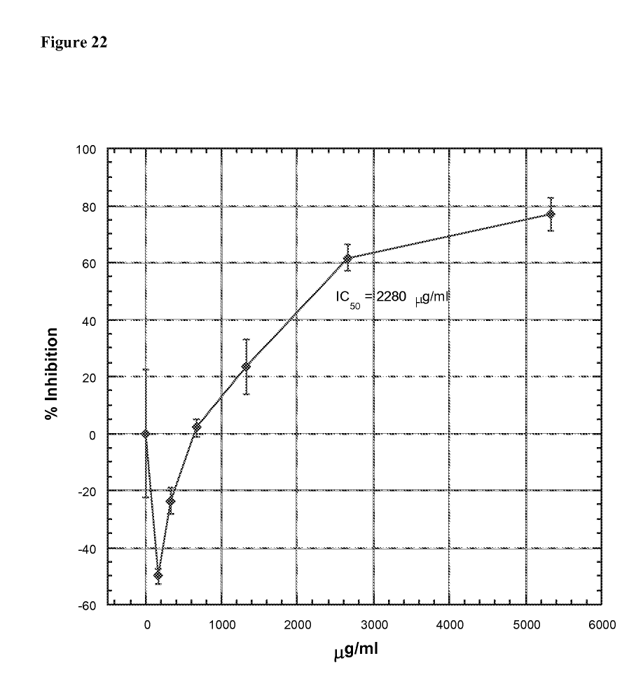

FIG. 23 depicts the infectivity inhibition dose response curve and 50% inhibitory concentration for buffered elderberry B anthocynin fraction ADS5 desorption F4 fraction using H1N1 virus.

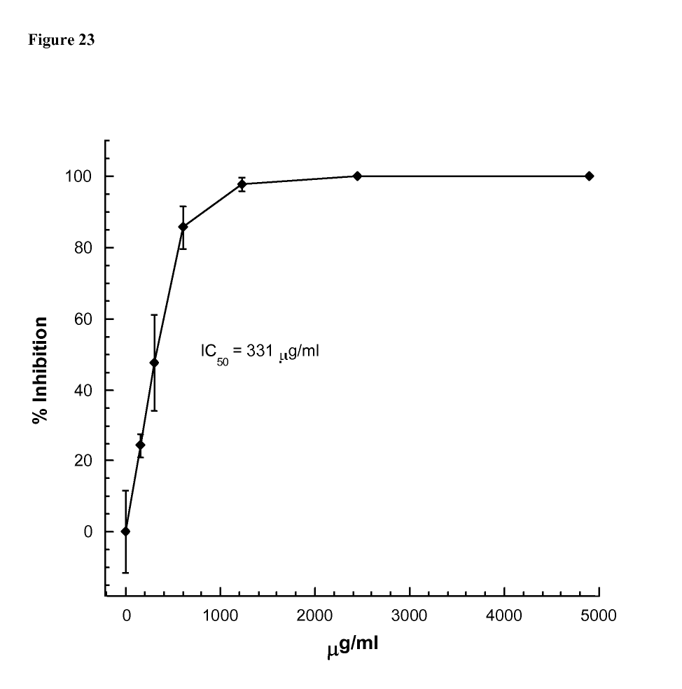

FIG. 24 depicts the infectivity inhibition dose response curve and 50% inhibitory concentration for unbuffered elderberry B anthocynin fraction ADS5 desorption F4 fraction using H1N1 virus.

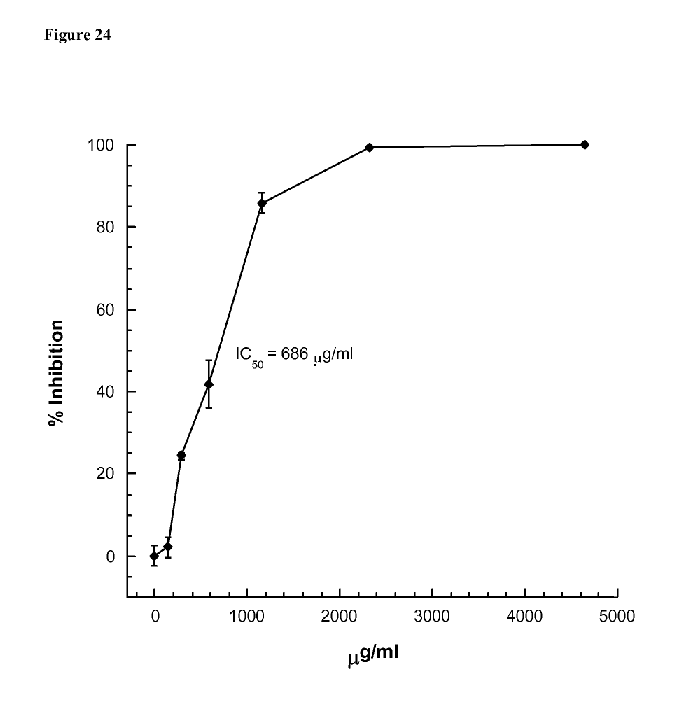

FIG. 25 depicts the infectivity inhibition dose response curve and 50% inhibitory concentration for buffered elderberry B anthocynin fraction ADS5 desorption F4 fraction using H5N1 virus.

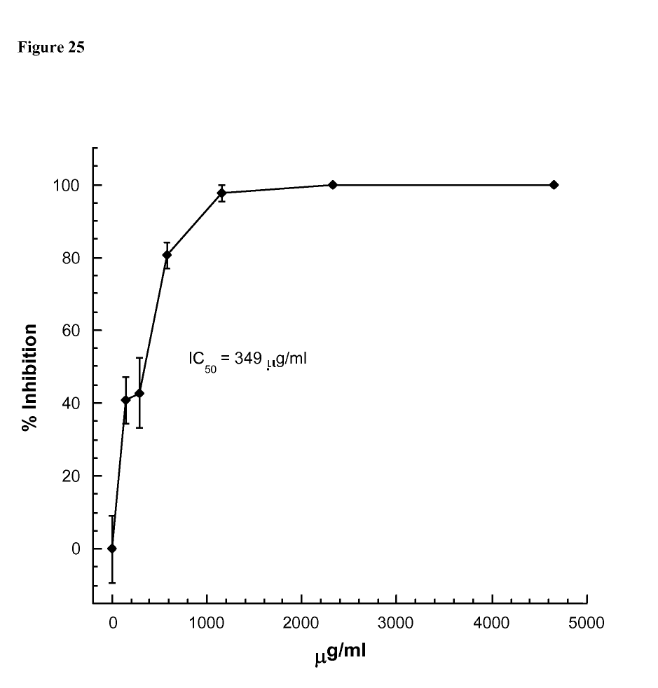

FIG. 26 depicts the infectivity inhibition dose response curve and 50% inhibitory concentration for buffered elderberry B anthocynin fraction ADS5 desorption F4 fraction using H5N1 virus.

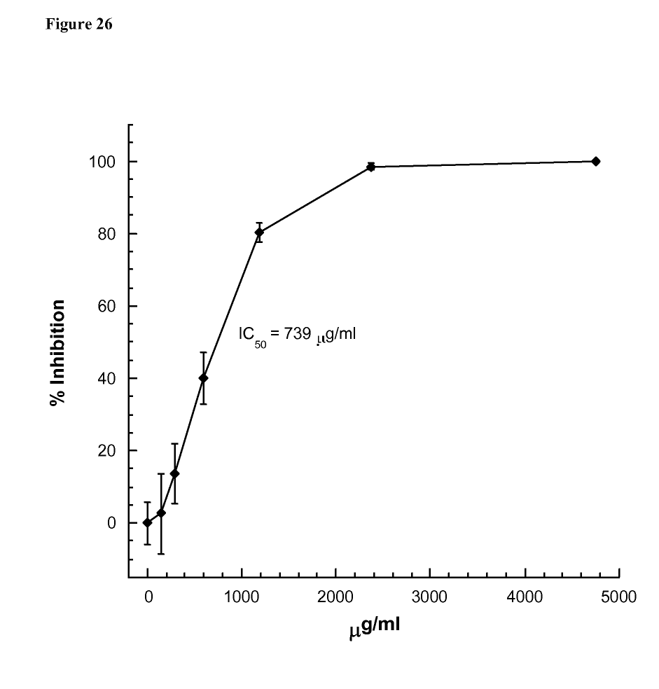

FIG. 27 depicts the combined infectivity inhibition dose response curves for tested extracts.

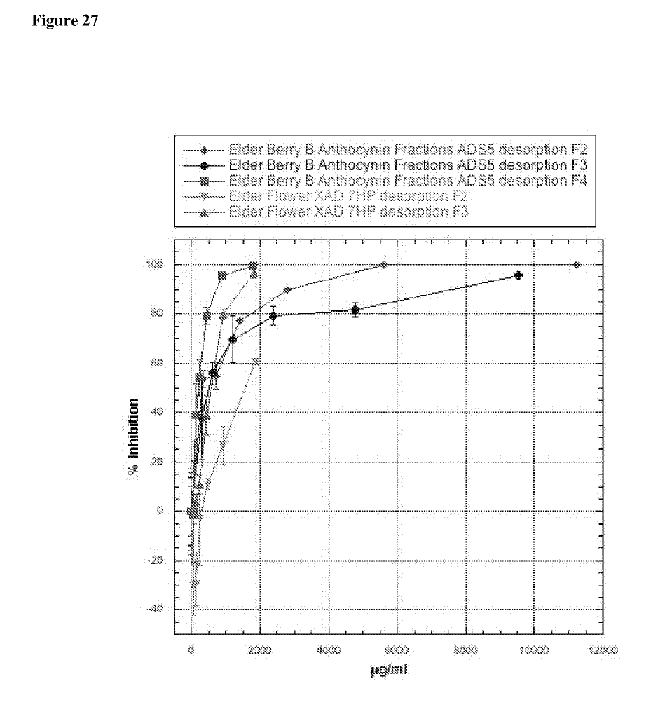

FIG. 28 depicts the infectivity inhibition dose response curve and 50% inhibitory concentration for buffered elder flower ADS5 desorption F2 fraction using H1N1 virus.

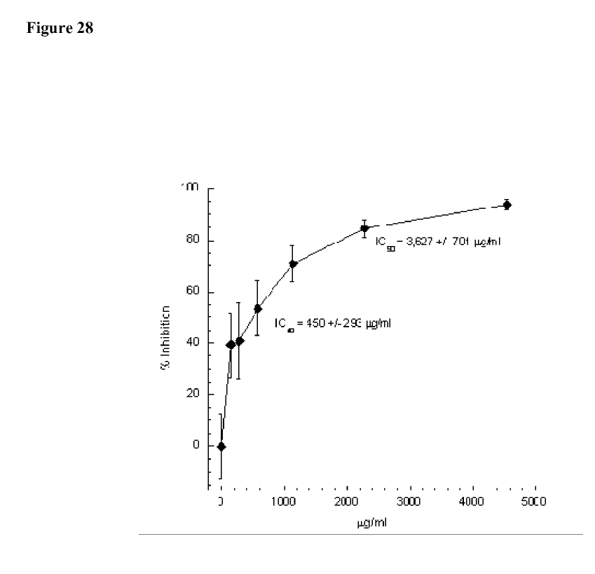

FIG. 29 depicts the calculated IC₅₀ H1N1 for elder flower F2 fraction.

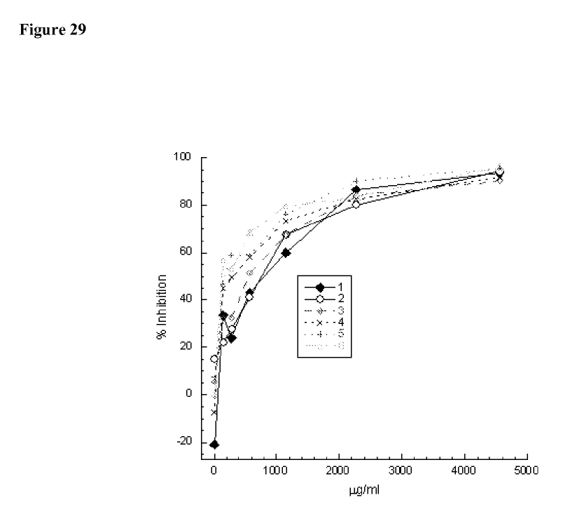

FIG. 30 depicts a comparison of IC₅₀ H1N1 for elderberry F4 fraction and elder flower F2 fraction.

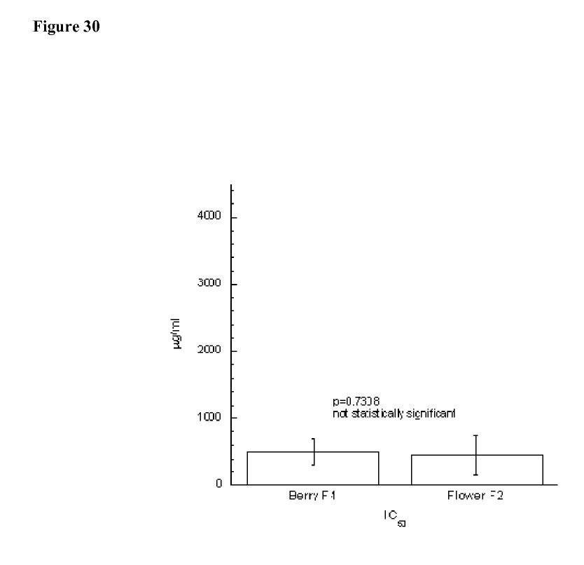

FIG. 31 depicts a comparison of IC₉₀ H1N1 for elderberry F4 fraction and elder flower F2 fraction.

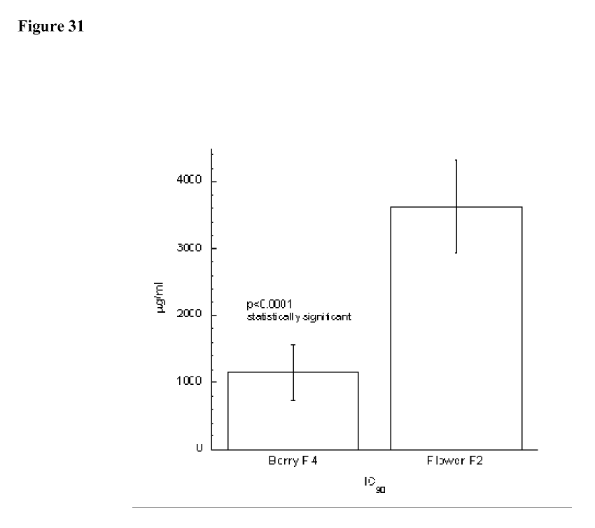

FIG. 32 depicts the infectivity inhibition dose response curve and 50% inhibitory concentration for elderberry B anthocynin fraction ADS5 desorption F2 using dengue virus type 2.

FIG. 33 depicts the infectivity inhibition dose response curve elderberry B anthocynin fraction ADS5 desorption F4 fraction using HIV virus. The curve shows 100% inhibition at the concentrations indicated.

FIG. 34 depicts the infectivity inhibition dose response curve elderberry B anthocynin fraction ADS5 desorption F4 fraction using HIV virus. The curve shows 100% inhibition at the concentrations indicated.

FIG. 35 depicts the infectivity inhibition dose response curve and 50% inhibitory concentration for elderberry B anthocynin fraction ADS5 desorption F4 fraction using HIV virus.

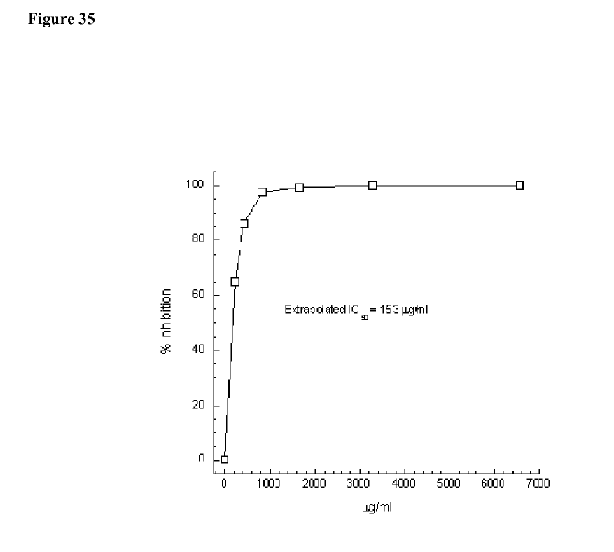

FIG. 36 depicts AccuTOF-DART Mass Spectrum for elderberry polysaccharide (positive ion mode).

FIG. 37 depicts AccuTOF-DART Mass Spectrum for elderberry polysaccharide (negative ion mode).

FIG. 38 depicts AccuTOF-DART Mass Spectrum for elder flower polysaccharide (positive ion mode).

FIG. 39 depicts AccuTOF-DART Mass Spectrum for elder flower polysaccharide (negative ion mode).

FIG. 40 depicts AccuTOF-DART Mass Spectrum for whole elderberry feedstock with plausible structures depicted (positive ion mode). Methyl cinnamic acid (163.0688) (abund.=19.47), cinnamide (148.0826) (abund.=2.63), 2-methoxyphenol (125.0599) (abund.=7.34), 3-methoxy-1-tyrosine (212.0985) (abund.=17.42), benzaldehyde (107.0422) (abund.=1.10), cinnamaldehyde (133.0568) (abund.=6.56), cinnamyl acetate (177.0956) (abund.=8.51), and pyrogallol (127.0344) (abund.=93.67) were detected. Unidentified compounds were also detected as C₆H₈O₄+H⁺ (at 145.0469) and C₆H₆O₃+H⁺ (at 127.0344).

FIG. 41 depicts AccuTOF-DART Mass Spectrum for whole elderberry feedstock with plausible structures depicted (negative ion mode). Cinnamic acid (147.0385) (abund.=5.57), cinnamaldehyde (131.04) (abund.=5.57), pyrogallol (125.024) (abund.=3.54), quercetin (301.0253) (abund.=0.73), ursolic acid (455.3518) (abund.=10.99), and shikimic acid (173.0454) (abund.=7.18) were detected.

FIG. 42 depicts AccuTOF-DART Mass Spectrum for an extraction of whole elderberry feedstock with an 80% EtOH solution (positive ion mode). Unidentified compounds were detected as C₆H₁₀O₅+H⁺ (163.0601) (abund.=17.19) and C₁₄H₁₅NO+H⁺ (214.1266) (abund.=24.06).

FIG. 43 depicts AccuTOF-DART Mass Spectrum for an F2 column chromatography fraction using ADS 5 desorption packing material (positive ion mode). Rutin or delphinidin (303.0541) (abund.=59.28), ferulic acid (195.0755) (abund.=13.54), cinnamic acid (149.0572) (abund.=19.55), shikimic acid (175.0699) (abund.=11.72), and phenyllacetic acid (167.0793) (abund.=36.17) were detected. Unidentified compounds were also detected as C₆H₆O₃+H⁺ (127.0348) (abund.=100) and C₇H₆O₄+H⁺ (155.0335) (abund.=59.18).

FIG. 44 depicts AccuTOF-DART Mass Spectrum for an F3 column chromatography fraction using ADS 5 desorption packing material (positive ion mode). Rutin or delphinidine (303.0521) (abund.=100), taxifolin (305.0693) (abund.=4.25), ferulic acid (195.075) (abund.=1.34), cinnamic acid (149.0552) (abund.=3.32), shikimic acid (175.0696) (abund.=0.96), phenyllacetic acid (167.0701) (abund.=3.97), cyanidin (287.0622) (abund.=8.36), and petunidin (317.0707) (abund.=21.71) were detected. Unidentified compounds were also detected as C₁₀H₁₂O₃+H⁺ (181.0854) (abund.=9.71) and C₁₃H₁₄N₂O₂+H⁺ (231.1163) (abund.=5.85).

FIG. 45 depicts AccuTOF-DART Mass Spectrum for an F4 column chromatography fraction using ADS 5 desorption packing material (positive ion mode). Rutin or delphinidine (303.0534) (abund.=100), ferulic acid (195.0744) (abund.=3.32), cinnamic acid (149.057) (abund.=6.36), cyanidin (287.0608) (abund.=36.44), petunidin (317.0691) (abund.=78.75), and vanillic acid (169.0524) (abund.=7.75) were detected. Unidentified compounds were also detected as C₂₉H₁₈O₇+H⁺ (479.1218) (abund.=22.62) and C₁₂H₁₄O₄+H⁺ (223.0994) (abund.=21.56).

FIG. 46 depicts AccuTOF-DART Mass Spectrum for an F2 column chromatography fraction using ADS 5 desorption packing material of elderberry B anthocyanin (positive ion mode). This fraction was used in an antiviral assay using H1N1 resulting in an IC₅₀=333 μg/mL. Rutin or delphinidine (303.0566) (abund.=18.33), ferulic acid (195.0724) (abund.=10.32), p-coumaric acid/phenylpyruvic acid (165.0639) (abund.=25.54), cinnamic acid (149.0573) (abund.=17.86), shikimic acid (175.0633) (abund.=12.62), and vanillic acid (169.0575) (abund.=18.01) were detected. Unidentified compounds were also detected as C₁₃H₁₁₀+H⁺ (183.0818) (abund.=43.33) and C₁₄H₁₇NO₃+H⁺ (248.1271) (abund.=60.28).

FIG. 47 depicts AccuTOF-DART Mass Spectrum for an F3 column chromatography fraction using ADS 5 desorption packing material of elderberry B anthocyanin (positive ion mode). This fraction was used in an antiviral assay using H1N1 resulting in an IC₅₀=294 μg/mL. Rutin or delphinidine (303.0553) (abund.=41.74), hesperin (287.0936) (abund.=10.41), cinnamic acid (149.0584) (abund.=17.85), and vanillic acid (169.0571) (abund.=31.09) were detected. Unidentified compounds were also detected as C₈H₈O+H⁺ (121.0586) (abund.=29.36) and C₁₄H₂₀N₂O₃+H⁺ or C₁₅H₂₀O₄+H⁺ (265.1469) (abund.=26.23).

FIG. 48 depicts AccuTOF-DART Mass Spectrum for an F4 column chromatography fraction using ADS 5 desorption packing material of elderberry B anthocyanin (positive ion mode). This fraction was used in an antiviral assay using H1N1 resulting in an IC₅₀=195 μg/mL. Rutin or delphinidine (303.0557) (abund.=20.27), cinnamic acid (149.0593) (abund.=7.94), petunidin (317.071) (abund.=22.09), and vanillic acid (169.0538) (abund.=12.82) were detected. Unidentified compounds were also detected as C₆H₁₀O₅+H⁺ (163.076) (abund.=63.28) and C₁₇H₁₈O+H⁺ (239.1531) (abund.=26.32).

FIG. 49 depicts AccuTOF-DART Mass Spectrum for an F2 column chromatography fraction using XAD 7HP desorption packing material of elder flower (positive ion mode). This fraction was used in an antiviral assay using H1N1 resulting in an IC₅₀=1,592 μg/mL. Cyanidin (287.0588) (abund.=10.92), rutin or delphinidine (303.0531) (abund.=100), taxifolin (305.0651) (abund.=4.69), caffeic acid/4-hydroxy phenylactic acid (181.0589) (abund.=9.45), ferulic acid (195.0741) (abund.=3.33), shikimic acid (175.0645) (abund.=3.11), petunidin (317.0689) (abund.=29.48), and eriodictyol or fustin (288.0709) (abund.=2.36) were detected. Unidentified compounds were also detected as C₁₀H₁₃NO₂+H⁺ (180.1024) (abund.=15.98) and C₈H₆N₂O+H or C₉H₆O₂+H⁺ (147.0545) (abund.=73.50).

FIG. 50 depicts AccuTOF-DART Mass Spectrum for an F3 column chromatography fraction using XAD 7HP desorption packing material of elder flower (positive ion mode). This fraction was used in an antiviral assay using H1N1 resulting in an IC₅₀=582 μg/mL. Cyanidin (287.0574) (abund.=17.16), rutin or delphinidine (303.0518) (abund.=100), taxifolin (305.0658) (abund.=5.54), naringenin/butein/phloretin (273.0797) (abund.=16.06), and eriodictyol or fustin (289.0795) (abund.=3.14) were detected. Unidentified compounds were also detected as C₁₀H₁₆O+H⁺ (153.1268) (abund.=30.96) and C₂₃H₁₄O₄+H⁺ (355.1048) (abund.=30.03).

FIG. 51 depicts AccuTOF-DART Mass Spectrum for #185 (positive ion mode). Cinnamic acid (149.0616) (abund.=3.82), shikimic acid (175.0613) (abund.=14.71), and phenyllacetic acid (167.074) (abund.=5.35) were detected. Unidentified compounds were also detected as C₃₀H₄₆O₂+H⁺ (439.3625) (abund.=16.49) and C₃₉H₆₈O₅+H⁺ (617.5151) (abund.=4.09).

FIG. 52 depicts AccuTOF-DART Mass Spectrum for #319 (positive ion mode). p-Coumaric acid/phenylpyruvic acid (165.0604) (abund.=3.96), cinnamic acid (149.0579) (abund.=0.48), 3,5-dimethoxy-4-hydroxy cinnamic acid (225.0816) (abund.=10.59), shikimic acid (175.0569) (abund.=5.37), and phenyllacetic acid (167.0773) (abund.=2.71) were detected. Unidentified compounds were also detected as C₆H₈O₄+H⁺ (145.0507) (abund.=100) and C₁₂H₁₂O₆+H⁺ (253.0708) (abund.=35.27).

FIG. 53 depicts AccuTOF-DART Mass Spectrum for #322 (positive ion mode). Delphinidin (304.0576) (abund.=8.75), rutin (303.057) (abund.=49.28), eriodictyol/fustin (289.0752) (abund.=13.50), taxifolin (305.0638) (abund.=3.41), ferulic acid (195.0745) (abund.=7.15), p-coumaric acid/phenylpyruvic acid (165.0613) (abund.=16.91), cinnamic acid (149.0695) (abund.=3.20), shikimic acid (175.067) (abund.=8.34), and phenyllacetic acid (167.0722) (abund.=8.84) were detected. Unidentified compounds were also detected as C₆H₆O₃+H⁺ (127.0413) (abund.=100) and C₁₁H₁₅O₅+H⁺ (227.0876) (abund.=29.26).

FIG. 54 depicts AccuTOF-DART Mass Spectrum for #324 (positive ion mode). Unidentified compounds were detected as C₃₇H₆₆O₄+H⁺ (575.51) (abund.=5.42) and C₅₉H₈₈O₅+H⁺ (877.67) (abund.=15.46).

FIG. 55 depicts AccuTOF-DART Mass Spectrum for #325 (positive ion mode). Shikimic acid (175.0658) (abund.=6.05) was detected. Unidentified compounds were also detected as C₁₆H₁₄O₄+H⁺ (271.0941) (abund.=22.24) and C₁₆H₁₆O₅+H⁺ (289.0983) (abund.=15.76).

FIG. 56 depicts AccuTOF-DART Mass Spectrum for #326 (positive ion mode). Cinnamic acid (149.0681) (abund.=2.67) was detected. Unidentified compounds were also detected as C₂₂H₄₂O₄+H⁺ (371.3196) (abund.=46.60) and C₁₈H₃₀O₂+H⁺ (279.2346) (abund.=20.28).

FIG. 57 depicts AccuTOF-DART Mass Spectrum for #327 (positive ion mode). Unidentified compounds were detected as C₈H₈O+H⁺ (121.0663) (abund.=66.34) and C₈H₈O₂+H⁺ (137.065) (abund.=20.16).

FIG. 58 depicts AccuTOF-DART Mass Spectrum for #328 (positive ion mode). Ferulic acid (195.0737) (abund.=4.04), p-coumaric acid/phenylpyruvic acid (165.0604) (abund.=3.67), cinnamic acid (149.0691) (abund.=3.49), 3,5-dimethoxy-4-hydroxy cinnamic acid (225.0817) (abund.=5.18), shikimic acid (175.0616) (abund.=4.88), and phenyllacetic acid (167.0786) (abund.=2.63) were detected. Unidentified compounds were also detected as C₆H₁₀O₅+H⁺ (163.0602) (abund.=10.84) and C₁₂H₁₄O₇+H⁺ (271.0829) (abund.=21.7).

FIG. 59 depicts AccuTOF-DART Mass Spectrum for #329 (positive ion mode). Cinnamic acid (149.0621) (abund.=1.43) and shikimic acid (175.0633) (abund.=3.23) were detected. Unidentified compounds were also detected as C₂₁H₃₆O₃+H⁺ (337.2763) (abund.=13.38) and C₃₉H₆₆O₄+H⁺ (599.507) (abund.=5.53).

FIG. 60 depicts AccuTOF-DART Mass Spectrum for #330 (positive ion mode). Ferulic acid (195.0747) (abund.=2.76), p-coumaric acid/phenylpyruvic acid (165.0608) (abund.=2.42), cinnamic acid (149.0616) (abund.=0.79), 3,5-dimethoxy-4-hydroxy cinnamic acid (225.0824) (abund.=2.98), shikimic acid (175.0604) (abund.=2.55), and phenyllacetic acid (167.078) (abund.=1.95) were detected. Unidentified compounds were also detected as C₁₄H₁₄O₄+H⁺ (247.0895) (abund.=4.28) and C₃₀H₄₆O₂+H⁺ (439.3619) (abund.=5.98).

FIG. 61 depicts AccuTOF-DART Mass Spectrum for #185 (negative ion mode). Hesperidin (285.0841) (abund.=0.44) and phloridzin (255.0711) (abund.=0.71) were detected. Unidentified compounds were also detected as C₄H₆O₅—H⁺ (133.0134) (abund.=100) and C₁₀H₈O₄—H⁺ (191.0325) (abund.=25.34).

FIG. 62 depicts AccuTOF-DART Mass Spectrum for #319 (negative ion mode). Cinnamic acid (147.0358) (abund.=0.67) was detected. Unidentified compounds were also detected as C₄H₆O₅—H⁺ (133.0135) (abund.=86.11) and C₁₀H₈O₄—H⁺ (191.0195) (abund.=100).

FIG. 63 depicts AccuTOF-DART Mass Spectrum for #322 (negative ion mode). Cyanidin (286.0502) (abund.=5.30), delphinidin (302.0388) (abund.=18.51), pelargonidin (270.0512) (abund.=0.34), myricetin (317.0315) (abund.=13.27), rutin (301.0324) (abund.=100), silybin/genistein (269.0399) (abund.=0.42), 3-OH flavone (237.0587) (abund.=0.89), eriodictyol/fustin (287.0592) (abund.=7.09), catechin/epitcatechin (289.0784) (abund.=5.29), taxifolin (303.0468) (abund.=5.31), phloridzin (255.0614) (abund.=0.81), vanillic acid (167.0416) (abund.=4.07), p-coumaric acid/phenylpyruvic acid (163.0307) (abund.=12.95), 3,5-dimethoxy-4-hydroxy cinnamic acid (223.054) (abund.=0.80), gallic acid (169.0166) (abund.=1.73), and shikimic acid (173.0475) (abund.=1.11) were detected. Unidentified compounds were also detected as C₁₀H₈O₄—H⁺ (191.0532) (abund.=31.51) and C₂₂H₂₂O₁₃—H⁺ (493.0955) (abund.=4.42).

FIG. 64 depicts AccuTOF-DART Mass Spectrum for #324 (negative ion mode). Eriodictyol/fustin (287.0655) (abund.=0.99), catechin/epitcatechin (289.0726) (abund.=0.92), ursolic acid (455.3465) (abund.=0.87), vanillic acid (167.0388) (abund.=1.89), ferulic acid (193.0478) (abund.=7.35), p-coumaric acid/phenylpyruvic acid (163.0404) (abund.=5.66), Cinnamic acid (147.0373) (abund.=5.97), and shikimic acid (173.0373) (abund.=10.00) were detected. Unidentified compounds were also detected as C₁₆H₁₄O₄—H⁺ (269.0878) (abund.=21.98) and C₂₃H₁₈O₃—H⁺ (341.1193) (abund.=12.27).

FIG. 65 depicts AccuTOF-DART Mass Spectrum for #325 (negative ion mode). Unidentified compounds were detected as C₄H₆O₅—H⁺ (133.0118) (abund.=100) and C₁₀H₈O₄—H⁺ (191.0183) (abund.=81.19).

FIG. 66 depicts AccuTOF-DART Mass Spectrum for #326 (negative ion mode). Rutin (301.0441) (abund.=31.62), 3-OH flavone (237.062) (abund.=0.74), catechin/epitcatechin (289.079) (abund.=2.70), phloridzin (255.0687) (abund.=2.24), ursolic acid (455.3556) (abund.=7.43), caffeic acid/4-hydroxyphenylactic acid (179.0398) (abund.=12.26), ferulic acid (193.051) (abund.=7.63), p-coumaric acid/phenylpyruvic acid (163.0405) (abund.=8.75), cinnamic acid (147.0414) (abund.=3.24), and shikimic acid (173.0452) (abund.=23.59) were detected. Unidentified compounds were also detected as C₅H₆O₄—H⁺ (129.0178) (abund.=100) and C₁₆H₁₆O₈—H⁺ (335.0807) (abund.=25.82).

FIG. 67 depicts AccuTOF-DART Mass Spectrum for #327 (negative ion mode). 3-OH flavone (237.0524) (abund.=0.26), hesperidin (285.0822) (abund.=0.63), catechin/epitcatechin (289.0732) (abund.=0.11), phloridzin (255.0706) (abund.=0.82), 3,5-dimethoxy-4-hydroxy cinnamic acid (223.0543) (abund.=0.09), and chorismic acid (225.0489) (abund.=0.10) were detected. Unidentified compounds were also detected as C₄H₆O₅—H⁺ (133.0117) (abund.=100) and C₂₀H₂₀O₇—H⁺ (371.1175) (abund.=2.39).

FIG. 68 depicts AccuTOF-DART Mass Spectrum for #328 (negative ion mode). Rutin (301.0446) (abund.=0.62), phloridzin (255.0744) (abund.=0.05), and p-coumaric acid/phenylpyruvic acid (163.0386) (abund.=0.36) were detected. Unidentified compounds were also detected as C₅H₈O₅—H⁺ (147.0293) (abund.=7.50) and C₆H₆O₆—H⁺ (173.0099) (abund.=7.84).

FIG. 69 depicts AccuTOF-DART Mass Spectrum for #329 (negative ion mode). Unidentified compounds were detected as C₆H₁₀O₅—H⁺ (161.04) (abund.=2.97) and C₈H₁₂O₇—H⁺ (219.05) (abund.=3.64).

FIG. 70 depicts AccuTOF-DART Mass Spectrum for #330 (negative ion mode). Unidentified compounds were detected as C₅H₄O₃—H⁺ (111.01) (abund.=12.32) and C₆H₁₂O₆—H⁺ (179.05) (abund.=1.20).

### DETAILED DESCRIPTION OF THE INVENTION

Definitions

The articles “a” and “an” are used herein to refer to one or to more than one (i.e. to at least one) of the grammatical object of the article. By way of example, “an element” means one element or more than one element.

The term “anthocyanidins” is art recognized and refers to compounds comprising flavylium cation derivatives.

The term “anthocyanins” is art recognized and refers to anthocyanidins with a sugar group. They are mostly 3-glucosides of the anthocyanidins. The anthocyanins are subdivided into sugar-free anthocyanidine aglycons and anthocyanin glycosides.

The term “capsid” is art recognized and refers to a protein coat that surrounds and protects the nucleic acid (DNA or RNA) of the virus.

The terms “comprise” and “comprising” are used in the inclusive, open sense, meaning that additional elements may be included.

The term “consisting” is used to limit the elements to those specified except for impurities ordinarily associated therewith.

The term “consisting essentially of” is used to limit the elements to those specified and those that do not materially affect the basic and novel characteristics of the material or steps.

The term “cyanidin” or “flavon-3-ol” is art recognized and refers to a natural organic compound classified as a flavonoid and an anthocyanin. It is a pigment found in many redberries including but not limited to bilberry, blackberry, blueberry, cherry, cranberry, elderberry, hawthorn, loganberry, acai berry and raspberry. It can also be found in other fruits such as apples and plums.

The term “effective amount” as used herein refers to the amount necessary to elicit the desired biological response. As will be appreciated by those of ordinary skill in this art, the effective amount of a composite or bioactive agent may vary depending on such factors as the desired biological endpoint, the bioactive agent to be delivered, the composition of the encapsulating matrix, the target tissue, etc.

As used herein, “elder” refers to the Sambucas plant material derived from the Sambucas species botanical. The term “elder” is also used interchangeably with elder species, Sambucas species, and elderberry and means these plants, clones, variants, and sports, etc.

As used herein, the term “elder constituents” shall mean chemical compounds found in elder species and shall include all such chemical compounds identified above as well as other compounds found in elder species, including but not limited to the essential oil chemical constituents, polyphenolic acids, and polysaccharides.

As used herein, the term “envelope virus” refers to a virus comprising a lipid bilayer containing viral glycoproteins derived from a host cell membrane. In an envelope viruse, viral proteins that mediate attachment and penetration into the host cell are found in the envelope. Examples of envelope viruses include influenza, both human and avian, HIV, SARs, HPV, herpes simplex virus (HSV), dengue, and flavie viruses, such as for example, yellow fever, West Nile, and encephalitis viruses.

As used herein, the term “essential oil fraction” comprises lipid soluble, water insoluble compounds obtained or derived from elder and related species including, but not limited to, the chemical compound classified as linoelaidic acid.

As used herein, the term “essential oil sub-fraction” comprises lipid soluble, water insoluble compounds obtained or derived from elder and related species including, but not limited to, the chemical compound classified as lineolaidic acid having enhanced or reduced concentrations of specific compounds found in the essential oil of elder species.

As used herein, “feedstock” generally refers to raw plant material, comprising whole plants alone, or in combination with on or more constituent parts of a plant comprising leaves, roots, including, but not limited to, main roots, tail roots, and fiber roots, stems, bark, leaves, berries, seeds, and flowers, wherein the plant or constituent parts may comprise material that is raw, dried, steamed, heated or otherwise subjected to physical processing to facilitate processing, which may further comprise material that is intact, chopped, diced, milled, ground or otherwise processed to affected the size and physical integrity of the plant material. Occasionally, the term “feedstock” may be used to characterize an extraction product that is to be used as feed source for additional extraction processes.

A “flavie virus” is a subset of envelope viruses. They are generally viruses found in animals that have infected humans by acquiring a lipid bilayer envelope. Examples of flavie viruses include yellow fever, dengue, West Nile, and encephalitis viruses.

As used herein, the term “fraction” means the extraction composition comprising a specific group of chemical compounds characterized by certain physical, chemical properties or physical or chemical properties.

The term “including” is used herein to mean “including but not limited to”. “Including” and “including but not limited to” are used interchangeably.

As used herein, the term “non-envelope virus” refers to a virus lacking a lipid bilayer. In non-envelope viruses the capsid mediates attachment to and penetration into host cells. Examples of non-envelope viruses include Norwalk virus, hepatitis A, polio, and rhinoviruses.

As used herein, the term “one or more compounds” means that at least one compound, such as, but not limited to, linoelaidic acid (a lipid soluble essential oil chemical constituent of elder species), or cyanidin-3-glucoside (a water soluble polyphenolic of elder species) or a polysaccharide molecule of elder species is intended, or that more than one compound, for example, linoelaidic acid and cyaniding-3-glucoside is intended. As known in the art, the term “compound” does not mean a single molecule, but multiples or moles of one or more compound. As known in the art, the term “compound” means a specific chemical constituent possessing distinct chemical and physical properties, whereas “compounds” refer to one or more chemical constituents.

A “patient,” “subject” or “host” to be treated by the subject method may be a primate (e.g. human), bovine, ovine, equine, porcine, rodent, feline, or canine.

The term “pharmaceutically-acceptable salts” is art-recognized and refers to the relatively non-toxic, inorganic and organic acid addition salts of compounds, including, for example, those contained in compositions of the present invention.

As used herein, the term “polyphenolic fraction” comprises the water soluble and ethanol soluble polyphenolic acid compounds obtained or derived from elder and related species, further comprising, but not limited to, compounds such as rutin, and cyaniding-3-glucoside.

As used herein, the term “polysaccharide fraction” comprises water soluble-ethanol insoluble lectin protein and polysaccharide compounds obtained or derived from elder and related species.

Other chemical constituents of elder may also be present in these extraction fractions.

The term “proanthocyanins” as used herein refers to dimers, trimers, and quadifers of anthocyanins.

As used herein, the term “profile” refers to the ratios by percent mass weight of the chemical compounds within an extraction fraction or sub-fraction or to the ratios of the percent mass weight of each of the three elder fraction chemical constituents in a final elder extraction composition.

As used herein, the term “purified” fraction or extraction means a fraction or extraction comprising a specific group of compounds characterized by certain physical-chemical properties or physical or chemical properties that are concentrated to greater than 10% by mass weight of the fraction's or extraction's chemical constituents. In other words, a purified fraction or extraction comprises less than 80% chemical constituent compounds that are not characterized by certain desired physical-chemical properties or physical or chemical properties that define the fraction or extraction.

The term “synergistic” is art recognized and refers to two or more components working together so that the total effect is greater than the sum of the components.

The term “treating” is art-recognized and refers to curing as well as ameliorating at least one symptom of any condition or disorder.

The term “virus” is art recognized and refers to non-cellular biological entities lacking metabolic machinery of their own and reproduce by using that of a host cell. Viruses comprise a molecule of nucleic acid (DNA or RNA) and can be envelope or non-envelope viruses.

Compositions

The present invention comprises compositions of isolated and purified fractions of essential oils (or essential oil sub-fractions), polyphenolic acids, and polysaccharides from one or more elder species. These individual fraction compositions can be combined in specific ratios (profiles) to provide beneficial combination compositions and can provide reliable or reproducible extract products that are not found in currently know extract products. For example, an essential oil fraction or sub-fraction from one species may be combined with an essential oil fraction or sub-fraction from the same or different species or with a polyphenolic acid fraction from the same or different species, and that combination may or may not be combined with a polysaccharide fraction from the same or different species of elder.

Extracted elder species composition may comprise any one, two, or all three of the concentrated extract fractions depending on the beneficial biological effect(s) desired for the given product. Typically, a composition containing all three elder species extraction fractions is generally desired as such novel compositions represent the first highly purified elder species extraction products that contain all three of the principal biologically beneficial chemical constituents found in the native plant material. Embodiments of the invention comprise methods wherein the predetermined characteristics comprise a predetermined selectively increased concentration of the elder species' essential oil chemical constituents, polyphenolic-anthocyanidins, and polysaccharides in separate extraction fractions.

In particular, the compositions of the present invention have elevated amounts of anthocyanins relative to known compositions including those found in nature. Anthocyanins are potent antioxidants, highly active chemicals that have been increasingly associated with a variety of health benefits, including protection against heart disease and cancer. In addition to their antioxidant properties, it has been reported that anthocyanins also may be used to treat diabetes, boosting insulin production by up to 50%. The compositions of the present invention may comprise elevated amounts of anthocyanins as the only active ingredient, or the compositions may contain other active ingredients associated with elder. Examples of other active ingredients include C16 or C18 fatty acids, alcohols, or esters found in the essential oil fraction, or a polysaccharide found in the polysaccharide fraction.

Anthocyanin and flavonoid can be concentrated and profiled by polymer adsorbent (PA) technology. Wide range of polymer adsorbent can be used in such application, such as Amberlite XAD4, XAD7HP (Rohm-Hass), Dialon HP20, HP21, SP825 (Mitsubishi), ADS 5, ADS17 (Naikai University). The operation principle of PA processing is based on “like attractive like” (whether the adsorbate will stay attached to the adsorbent or dissolve into the eluent depends upon the relative strength). Examples of using XAD7HP and ADS5 are presented herein. The results are shown in the following tables:

TABLE 3
Weight % of anthocyanin components post extraction.

| | | | | | | | ADS5 Polymer | | |
|---|---|---|---|---|---|---|---|---|---|
| | XAD7HP Polymer Adsorbent | | | | | | Adsorbent | | |
| | Feed | F2 | F3 | F4 | F5 | F6 | F2 | F3 | F4 |
| Total Anthocyanin | 0.06 | 2.43 | 2.99 | 2.92 | 1.29 | 0.80 | 2.2 | 0.03 | 0.003 |
| CY-3,5-GLU | 0.02 | 1.04 | 0.83 | 0.56 | 0.06 | 0.07 | 0.8 | 0 | |
| CY-3-SAM | 0.01 | 0.37 | 0.48 | 0.45 | 0.22 | 0.14 | 0.33 | 0 | |
| CY-3-GLU | 0.04 | 1.01 | 1.67 | 1.91 | 1.01 | 0.59 | 1.06 | 0.03 | 0.003 |
| Rutin | 0.27 | 0.23 | 2.60 | 5.74 | 16.28 | 17.01 | 0.41 | 29.12 | 11.2 |
| Total Phenolic Acid | 1.55 | 27.81 | 31.02 | 40.49 | 31.87 | 36.87 | 41.8 | 34.3 | 20.7 |

TABLE 4
Anthocyanin profile.

| | | | | | | | ADS5 Polymer | | |
|---|---|---|---|---|---|---|---|---|---|
| | XAD7HP Polymer Adsorbent | | | | | | Adsorbent | | |
| | Feed | F2 | F3 | F4 | F5 | F6 | F2 | F3 | F4 |
| Total | 88.1 | 100.0 | 100.0 | 100.0 | 100.0 | 100.0 | 100 | 100 | 100 |
| Antho- | | | | | | | | | |
| cyanin | | | | | | | | | |
| CY-3,5- | 25.8 | 43.0 | 27.9 | 19.2 | 4.8 | 9.1 | 36.6 | 11.5 | |
| GLU | | | | | | | | | |
| CY-3- | 9.0 | 15.3 | 16.1 | 15.5 | 16.8 | 16.9 | 15 | 11.1 | |
| SAM | | | | | | | | | |
| CY-3- | 53.2 | 41.8 | 56.0 | 65.3 | 78.3 | 73.9 | 48.4 | 77.4 | 100 |
| GLU | | | | | | | | | |

TABLE 5
Ratio of rutin to total anthocyanin.

| | | | | | | | ADS5 Polymer | | |
|---|---|---|---|---|---|---|---|---|---|
| | XAD7HP Polymer Adsorbent | | | | | | Adsorbent | | |
| | Feed | F2 | F3 | F4 | F5 | F6 | F2 | F3 | F4 |
| Ration | 3.8 | 0.10 | 0.87 | 1.96 | 12.58 | 21.31 | 0.20 | 885 | 3267 |
| or rutin | | | | | | | | | |
| to total | | | | | | | | | |
| antho- | | | | | | | | | |
| cyanin | | | | | | | | | |

TABLE 6
Profile %.

| | | | | | | | ADS5 Polymer | | |
|---|---|---|---|---|---|---|---|---|---|
| | XAD7HP Polymer Adsorbent | | | | | | Adsorbent | | |
| | Feed | F2 | F3 | F4 | F5 | F6 | F2 | F3 | F4 |
| Total | 4.6 | 8.7 | 9.6 | 7.2 | 4.1 | 2.2 | 5.3 | 0.1 | 0.02 |
| Anthocyanin | | | | | | | | | |
| CY-3,5-GLU | 1.2 | 3.8 | 2.7 | 1.4 | 0.2 | 0.2 | 1.9 | 0 | |
| CY-3-SAM | 0.4 | 1.3 | 1.5 | 1.1 | 0.7 | 0.4 | 0.8 | 0 | |
| CY-3-GLU | 2.5 | 3.6 | 5.4 | 4.7 | 3.2 | 1.6 | 2.5 | 0.1 | 0.02 |
| Rutin | 17.6 | 0.8 | 8.4 | 14.2 | 51.1 | 46.2 | 1 | 84.9 | 54.1 |

The weight percentage of compounds tell us how much the compounds has been purified (concentrated) during processing: cyanidin-3,5-glucoside has been purified to up to 56.2 fold of that in feedstock (F2, XAD7HP PA); cyanidin-3-sambubioside has been purified to up to 74 fold of that in feedstock (F3, XAD7HP PA); Cyanidin-3-glucoside has been purified to up to 50 fold of that in feedstock (F4, XAD7HP); total anthocyanin has been purified up to 46-47 fold of that in feedstock (F2 and F3, XAD 7HP PA); rutin has been purified to 107 fold of that in feedstock (F3, ADS5 PA) and total phenolic acid has been purified to 13-17 fold of that of feedstock.

The anthocyanin profile data show that the profile of anthocyanin can be tuned during processing: cyanidin-3-glucoside can be profiled between 42%-100%; cyanidin-3-sambubioside can be profiled between 9%-17%; and cyaniding-3,5-glucoside can be profiled between 4.8%-43%.

Rutin and anthocyanin are important pharmaceutical compounds in elder species. The ratio of rutin vs. total anthocyanin can be profiled between 0.10-3267 during processing.

Anthocyanin and rutin concentration in total phenolic acid can also be profiled during processing: cyanidin-3-glucoside can be profiled between 0.02-5.4%; cyanidin-3-sambubioside can be profiled between 0-1.5%; cyaniding-3,5-glucoside can be profiled between 0-3.8%; total anthocyanin can be profiled between 0.02-9.6%; and rutin can be profiled between 0.8-84.9%.

In one embodiment, the compositions of the present invention contain elevated amounts of anthocyanins and a pharmaceutical carrier as discussed below. In another embodiment, the compositions of the present invention comprise another elder species such as C16 and C18 saturated and unsaturated fatty acids, alcohols and esters from the essential oil fraction.

The comparison between literature data of volatile constituents of dry elder flowers (Toulemonde 1983) and current research are shown in the following table:

TABLE 7
Comparison of literature and experimental data.

| | Literature Data | | | | | | | | | |
|---|---|---|---|---|---|---|---|---|---|---|
| | | iso- | | | | | | | | |
| Fatty | essential | pentane | ethanol | Experimental Data | | | | | | |
| Acid | oil | extract | concentrate | T4P1 | T4P3 | T4P5 | T6P3 | T6P5 | T8P3 | T8P5 |
| undecanoic | 3 | | | 0.06 | 0.19 | 0.35 | 2.78 | 0 | 0.87 | 1.6 |
| dedecanoic | 2 | | | 0.07 | 0.31 | 0.3 | 1.02 | 0.87 | 0.74 | 0.92 |
| myristic | 2.1 | 0.4 | 1 | 0.17 | 0.15 | 0 | 0 | 0.12 | 0.29 | 0.5 |
| penta- | 0.8 | 0.2 | 1.2 | 0.13 | 0.29 | 0 | 0.92 | 0.42 | 0 | 0 |
| decanoic | | | | | | | | | | |
| palmitic | 37.8 | 16.6 | 19.4 | 20.57 | 15.04 | 11.91 | 11.79 | 22.36 | 22.64 | 21.39 |
| stearic | 0.4 | 0.7 | 0.7 | 6.36 | 5.95 | 4.63 | 3.53 | 5.58 | 7.51 | 4.56 |
| oleic | 5.7 | 7.9 | 18 | 5.91 | 8.82 | 6.08 | 8.54 | 9.14 | 12.71 | 8.01 |
| linoleic | 9 | 17.5 | 19 | 31.99 | 5.24 | 11.33 | 5.65 | 2.87 | 11.93 | 4.89 |
| linolenic | 9.1 | 24 | 16 | 2.73 | 2.12 | 0 | 0 | 1.88 | 2.3 | 1.31 |
| linoelaidic | | | | 22.11 | 8.5 | | 1.33 | 1.52 | 2.38 | 1.19 |
| Total Fatty | 69.9 | 67.3 | 75.3 | 71.41 | 17.81 | 17.54 | 9.77 | 8.82 | 2.62 | 10.08 |
| Acid | | | | | | | | | | |

The compositions of the present invention may comprise elevated amounts of anthocyanins and a polysaccharide. In the water crude extracts, the protein yield were 0.09% in elder flower and 0.59% in elderberry. 95% of protein in crude extract can be precipitate by 80% ethanol. Therefore, 80% precipitates are polysaccharide-protein complex. The average molecular weight of these complexes were ˜2000 KDa. In one embodiment, the composition comprises a lectin-polysaccharide fraction composition, having a purity of 100-170 mg/g dextran equivalence based on the colormetric analytical methods and lectin protein purity of greater than 4-50% by mass weight based on the Bradford protein assay as taught in the present invention.

The compositions of the present invention may comprise elevated amounts of anthocyanins, C16 or C18 saturated or unsaturated fatty acid, alcohol, and a polysaccharide.

Extractions Relative to Natural Elder Species

Compositions of the present invention may also be defined in terms of concentrations relative to those found in natural elder species. For example, concentration of essential oils is from 0.001 to 10000 times the concentration of native elder species, and/or compositions where the concentration of desired polyphenolic acids is from 0.001 to 40 times the concentration of native elder species, and/or compositions where the concentration of water soluble-ethanol insoluble polysaccharides is from 0.001 to 40 times the concentration of native elder species, and/or composition wherein the concentration of lectin proteins is from 0.001 to 100 times the concentration of native elder species plant material. Compositions of the present invention comprise compositions wherein the concentration of essential oils is from 0.01 to 10000 times the concentration of native elder species, and/or compositions wherein the concentration of desired polyphenolic acids is from 0.01 to 40 times the concentration of native elder species, and/or compositions wherein the concentration of polysaccharides is from 0.01 to 40 times the concentration of native elder species, and/or composition wherein the concentration of lectin proteins is from 0.01 to 100 times the concentration of native elder species plant material. Furthermore, compositions of the present invention comprise sub-fractions of the essential oil chemical constituents having at least one or more of chemical compounds present in the native plant material essential oil that is in amount greater than or less than that found in native elder plant material essential oil chemical constituents. For example, the chemical compound, lineolaidic acid, may have its concentration increased in an essential oil sub-fraction to 22% by % mass weight of the sub-fraction from its concentration of 2% by % mass weight of the total essential oil chemical constituents in the native elder plant material, a 10 fold increase in concentration. In contrast, lineolaidic acid may have it's concentration reduced in an essential oil sub-fraction to less than 0.01% by % mass weight of the sub-fraction from it's concentration of about 2% by % mass weight of the total essential oil chemical constituents in the native plant material, a 100 fold decrease in concentration. Compositions of the present invention comprise compositions wherein the concentration of specific chemical compounds in such novel essential oil sub-fractions is either increase by about 1.1 to about 10 times or decreased by about 0.1 to about 100 times that concentration found in the native elder essential oil chemical constituents.

Purity of the Extractions

In performing the previously described extraction methods, it was found that greater than 80% yield by mass weight of the essential oil chemical constituents having greater than 95% purity of the essential oil chemical constituents in the original dried berry or flower feedstock of the elder species can be extracted in the essential oil SCCO₂ extract fraction (Step 1A). Using the methods as taught in Step 1A and 1B, the essential oil yield may be reduced due to the sub-fractionation of the essential oil chemical constituents into highly purified essential oil sub-fractions having novel chemical constituent profiles. In addition, the SCCO₂ extraction and fractionation process as taught in this invention permits the ratios (profiles) of the individual chemical compounds comprising the essential oil chemical constituent fraction to be altered such that unique essential oil sub-fraction profiles can be created for particular medicinal purposes. For example, the concentration of the alcohol essential oil chemical constituents may be increased while simultaneous reducing the concentration of the fatty acid compounds or visa versa.

Using the methods as taught in Step 2 of this invention, a hydroalcoholic leaching fraction is achieved with a 35.6% mass weight yield from the original elder species feedstock having a 4.3% concentration of total phenolic acids, a yield of about 60% mass weight of the phenolic acid chemical constituents found in the native elderberry feedstock. Furthermore, this hydroalcoholic solvent extract also contains the valuable anthocyanidin chemical constituents.

Using the methods as taught in Step 3 of this invention (Affinity Adsorbent Extraction Processes or Process Chromatography), polyphenolic acid fractions with purities of greater than 40% by % dry mass of the extraction fraction with greater than 2.5% anthocyanidins by % mass weight may be obtained. It is possible to extract about 60% of the polyphenolic acids from the hydroalcoholic leaching extract feedstock. This equates to a 40% yield of the polyphenolic acid chemical constituents found in the native elder species plant material. It is also possible to produce purified phenolic acid sub-fractions that contain high concentrations of phenolic acids (>30% mass weight) with either relatively high concentrations of anthocyanidins (>2.9% mass weight) or low concentrations of anthocyanidins (<0.05% mass weight).

Using the methods as taught in Step 4 of the invention (water leaching and ethanol precipitation, it appears that greater than 90% yield by % mass weight of the water soluble-ethanol insoluble lectin protein and polysaccharide chemical constituents of the original dried elder species feedstock material can be extracted and purified in the lectin-polysaccharide fraction. Using 80% ethanol to precipitate the lectins and polysaccharides, a purified lectin-polysaccharide fraction may be collected from the water leaching extract. The yield of the lectin-polysaccharide fraction is about 3.45% by % mass weight based on the native elder plant material feedstock. Based on a colormetric analytical method using dextran as reference standards, a polysaccharide purity of 100-170 mg/gm dextran equivalents may be obtained. Based on the Bradford protein assay, a lectin purity of 16% by mass weight of the fraction may be obtained. Available evidence would indicate that the remaining compounds in the fraction are the polysaccharides (about 83% by mass weight). The purity of the lectin proteins can be reduced to about 5% using 60% ethanol precipitation or may be further increase to about 50% by mass weight of a sub-fraction using a staged 80% ethanol precipitation of the residue solution after a 60% ethanol precipitation and extraction of the polysaccharides.

Finally, the methods as taught in the present invention permit the purification (concentration) of the elder species essential oil chemical constituent fractions, novel polyphenolic fractions or sub-fractions, and novel lectin-polysaccharide fractions to be as high as 99% by mass weight of the desired chemical constituents in the essential oil fractions, as high as 41% by mass weight of the phenolic acids in the phenolic fraction, as high as 3% of the anthocyanidins in the polyphenolic fraction, as high as 50% of lectins by mass weight in the lectin-polysaccharide fraction, and as high as 90% polysaccharides by mass weight in the lectin-polysaccharide fraction. The specific extraction environments, rates of extraction, solvents, and extraction technology used depend on the starting chemical constituent profile of the source material and the level of purification desired in the final extraction products. Specific methods as taught in the present invention can be readily determined by those skilled in the art using no more than routine experimentation typical for adjusting a process to account for sample variations in the attributes of starting materials that is processed to an output material that has specific attributes. For example, in a particular lot of elder species plant material, the initial concentrations of the essential oil chemical constituents, the polyphenolic acids, the anthocyanidins, the lectins, and the polysaccharides are determined using methods known to those skilled in the art as taught in the present invention. One skilled in the art can determine the amount of change from the initial concentration of the essential oil chemical constituents, for instance, to the predetermined amounts or distribution (profile) of essential oil chemical constituents for the final extraction product using the extraction methods, as disclosed herein, to reach the desired concentration and/or chemical profile in the final elder species composition product.

Subfractions

A further embodiment of the invention is compositions comprising novel sub-fractions of the essential oil chemical constituents wherein the concentration of specific chemical groups such as, but not limited to, alcohols, aldehydes, esters or fatty acids have their respective concentrations increased for decreased in novel extraction composition products.

Another embodiment of the invention is compositions comprising novel sub-fractions of the purified polyphenolic chemical constituents wherein the concentration of specific chemical groups such as, but not limited to, anthocyanidins have their respective concentrations increased or decreased in novel extraction compositions.

An additional embodiment of the invention is compositions comprising novel sub-fractions of the purified lectin-polysaccharide chemical constituents wherein the concentration of specific chemical groups such as, but not limited to, lectins have respective concentrations increased or decreased in novel extraction compositions.

Methods of Extraction

Methods of the present invention provide novel elder compositions for the treatment and prevention of human disorders. For example, a novel elder species composition for treatment of influenza may have an increased polyphenolic fraction composition concentration, an increased polysaccharide composition concentration, and reduced essential oil fraction composition concentrations, by % weight, than that found in the elder species native plant material or conventional known extraction products. A novel elder species composition for anti-oxidant, anti-blood vessel damage, and ischemic cerebrovascular disease may have an increased essential oil and polyphenolic acid fraction composition and a reduced polysaccharide fraction composition, by % weight, than that found in the native elder species plant material or conventional known extraction products. Another example of a novel elder species composition, for treatment of diabetic disorders comprises a composition having an increased polyphenolic fraction composition concentration, a reduced polysaccharide composition, and a reduced essential oil fraction composition than that found in native elder species plant material or known conventional extraction products.

Additional embodiments comprise compositions comprising altered profiles (ratio distribution) of the chemical constituents of the elder species in relation to that found in the native plant material or to currently available elder species extract products. For example, the essential oil fraction may be increased or decreased in relation to the polyphenolic acids and/or polysaccharide concentrations. Similarly, the polyphenolic acids or polysaccharides may be increased or decreased in relation to the other extract constituent fractions to permit novel constituent chemical profile compositions for specific biological effects. By combining the isolated and purified fractions of one or more of essential oils, polyphenolics and/or polysaccharides, novel compositions may be made.

The following methods as taught may be used individually or in combination with the disclosed method or methods known to those skilled in the art.

The starting material for extraction is plant material from one or more elder species. The plant material may be the any portion of the plant, though the berry and flower are the most preferred starting material.

The elder species plant material may undergo pre-extraction steps to render the material into any particular form, and any form that is useful for extraction is contemplated by the present invention. Such pre-extraction steps include, but are not limited to, that wherein the material is chopped, minced, shredded, ground, pulverized, cut, or torn, and the starting material, prior to pre-extraction steps, is dried or fresh plant material. A preferred pre-extraction step comprises grinding and/or pulverizing the elder species plant material into a fine powder. The starting material or material after the pre-extraction steps can be dried or have moisture added to it. Once the elder species plant material is in a form for extraction, methods of extraction are contemplated by the present invention.

Supercritical Fluid Extraction of Elder

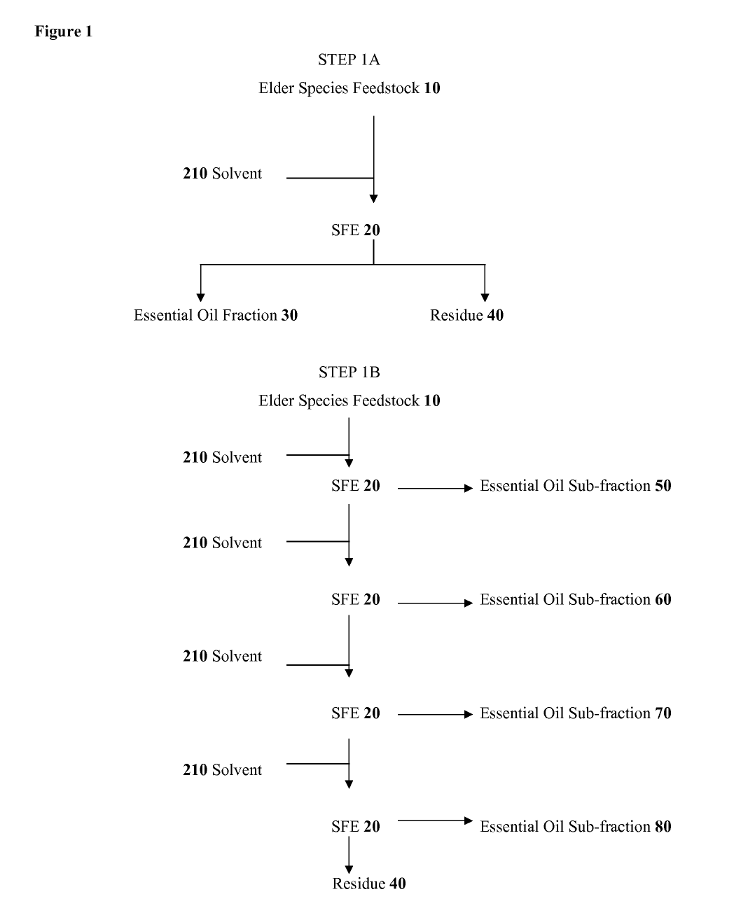

Methods of extraction of the present invention comprise processes disclosed herein. In general, methods of the present invention comprise, in part, methods wherein elder species plant material is extracted using supercritical fluid extraction (SFE) with carbon dioxide as the solvent (SCCO₂) that is followed by one or more solvent extraction steps, such as, but not limited to, water, hydroalcoholic, and affinity polymer absorbent extraction processes. Additional other methods contemplated for the present invention comprise extraction of elder species plant material using other organic solvents, refrigerant chemicals, compressible gases, sonification, pressure liquid extraction, high speed counter current chromatography, molecular imprinted polymers, and other known extraction methods. Such techniques are known to those skilled in the art. In one aspect, compositions of the present invention may be prepared by a method comprising the steps depicted schematically in FIGS. 1-4.

The invention includes processes for concentrating (purifying) and profiling the essential oil and other lipid soluble compounds from elder plant material using SCCO₂ technology. The invention includes the fractionation of the lipid soluble chemical constituents of elder into, for example, an essential oil fraction of high purity (high essential oil chemical constituent concentration). Moreover, the invention includes a SCCO₂ process wherein the individual chemical constituents within an extraction fraction may have their chemical constituent ratios or profiles altered. For example, SCCO₂ fractional separation of the chemical constituents within an essential oil fraction permits the preferential extraction of certain essential oil compounds relative to the other essential oil compounds such that an essential oil extract sub-fraction can be produced with a concentration of certain compounds greater than the concentration of other compounds. Extraction of the essential oil chemical constituents of the elder species with SCCO₂ as taught in the present invention eliminates the use of toxic organic solvents and provides simultaneous fractionation of the extracts. Carbon dioxide is a natural and safe biological product and an ingredient in many foods and beverages.

A schematic diagram of the methods of extraction of the biologically active chemical constituents of elder is illustrated in FIGS. 1-4. The extraction process is typically, but not limited to, 5 steps. The analytical methods used in the extraction process are presented in the Exemplification section.

Step 1: Supercritical Fluid Carbon Dioxide Extraction of Elder Essential Oil

Due to the hydrophobic nature of the essential oil, non-polar solvents, including, but not limited to SCCO₂, hexane, petroleum ether, and ethyl acetate may be used for this extraction process. Since some of the components of the essential oil are volatile, steam distillation may also be used as an extraction process.

A generalized description of the extraction of the essential oil chemical constituents from the rhizome of the elder species using SCCO₂ is diagrammed in FIG. 1. The feedstock 10 is dried ground elderberry or flower (about 140 mesh). The extraction solvent 210 is pure carbon dioxide. Ethanol may be used as a co-solvent. The feedstock is loaded into a into a SFE extraction vessel 20. After purge and leak testing, the process comprises liquefied CO₂ flowing from a storage vessel through a cooler to a CO₂ pump. The CO₂ is compressed to the desired pressure and flows through the feedstock in the extraction vessel where the pressure and temperature are maintained at the desired level. The pressures for extraction range from about 60 bar to 800 bar and the temperature ranges from about 35° C. to about 90° C. The SCCO₂ extractions taught herein are preferably performed at pressures of at least 100 bar and a temperature of at least 35<C, and more preferably at a pressure of about 60 bar to 500 bar and at a temperature of about 40° C. to about 80° C. The time for extraction for a single stage of extraction range from about 30 minutes to about 2.5 hours, to about 1 hour. The solvent to feed ratio is typically about 60 to 1 for each of the SCCO₂ extractions. The CO₂ is recycled. The extracted, purified, and profiled essential oil chemical constituents 30 are then collected a collector or separator, saved in a light protective glass bottle, and stored in a dark refrigerator at 4° C. The elder feedstock 10 material may be extracted in a one step process (FIG. 1) wherein the resulting extracted and purified elder essential oil fraction 30 is collected in a one collector SFE or SCCO₂ system 20 or in multiple stages (FIG. 1, Step 1B) wherein the extracted purified and profiled elder essential oil sub-fractions 50, 60, 70, 80 are separately and sequentially collected in a one collector SFE system 20. Alternatively, as in a fractional SFE system, the SCCO₂ extracted elder feedstock material may be segregated into collector vessels (separators) such that within each collector there is a differing relative percentage essential oil chemical constituent composition (profile) in each of the purified essential oil sub-fractions collected. The residue (remainder) 40 is collected, saved and used for further processing to obtain purified fractions of the elder species phenolic acids and polysaccharides. An embodiment of the invention comprises extracting the elder species feedstock material using multi-stage SCCO₂ extraction at a pressure of 60 bar to 500 bar and at a temperature between 35° C. and 90° C. and collecting the extracted elder material after each stage. A second embodiment of the invention comprises extracting the elder species feedstock material using fractionation SCCO₂ extraction at pressures of 60 bar to 500 bar and at a temperature between 35<C and 90<C and collecting the extracted elder material in differing collector vessels at predetermined conditions (pressure, temperature, and density) and predetermined intervals (time). The resulting extracted elder purified essential oil sub-fraction compositions from each of the multi-stage extractors or in differing collector vessels (fractional system) can be retrieved and used independently or can be combined to form one or more elder essential oil compositions comprising a predetermined essential oil chemical constituent concentration that is higher or lower than that found in the native plant material or in conventional elder extraction products. Typically, the total yield of the essential oil fraction from elder species berries using a single step maximal SCCO₂ extraction is about 9% (>95% of the essential oil chemical constituents) by % weight having an essential oil chemical constituent purity of greater than 95% by mass weight of the extract. In contrast, the total yield of the essential oil fraction from elder flowers using a single step maximal SCCO₂ extraction is about 1.5% (>95% of the essential oil chemical constituents) by % mass weight having an essential oil chemical constituent purity of greater than 95% by mass weight of the extract. These data demonstrate that the elderberries contain about 6 times the concentration of essential oil compounds than does the flowers. For examples of the present invention, the elderberries were used as the native elder species feedstock material. An example of this extraction process can be found in Example 1.

In this experimental example using elderberry as the feedstock, the extraction conditions were set wherein the temperatures ranges from 40-80<C and the pressures ranges from 80-500 bar. The CO₂ flow rate was 10 gm/min. The results are shown in Tables 8 and 9.

TABLE 8
Effects of temperature, pressure, and time on SCCO₂ essential oil
extraction yield using elderberry as feedstock.

| | T = 40 C. | | | T = 60 C. | | | T = 80 C. | | |
|---|---|---|---|---|---|---|---|---|---|
| | P (bar) | | | | | | | | |
| | 100 | 300 | 500 | 100 | 300 | 500 | 100 | 300 | 500 |
| | Density (g/cc) | | | | | | | | |
| Time | 0.64 | 0.915 | 1.00 | 0.297 | 0.834 | 0.94 | 0.227 | 0.751 | 0.88 |
| (min) | YIELD (%) | | | | | | | | |
| 5 | 0.00 | 3.68 | | | 1.49 | 1.34 | | 0.00 | 3.21 |
| 10 | 0.52 | 6.13 | 6.71 | | 4.68 | 5.57 | | 2.67 | 7.58 |
| 15 | 0.54 | 6.78 | | | 7.05 | 7.56 | | 4.34 | 8.69 |
| 20 | 0.67 | 7.92 | 7.00 | | 7.95 | 8.27 | | 5.93 | 9.57 |
| 30 | 1.11 | 8.42 | 7.12 | | 8.35 | 8.81 | | 8.19 | 9.79 |
| 60 | 1.53 | 8.63 | 7.51 | 0.60 | 8.53 | 9.39 | 0.45 | 8.85 | 9.86 |
| 90 | 2.09 | 8.98 | 7.63 | | 8.71 | 9.43 | | | |
| 120 | 2.10 | 9.31 | | | | | | | |

TABLE 9
GC-MS chemical compositions of elderberry SCCO₂ essential oil
extraction fractions extracted at different SFE conditions (temperature-T
and pressure in bar).

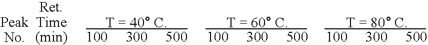

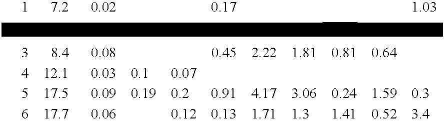

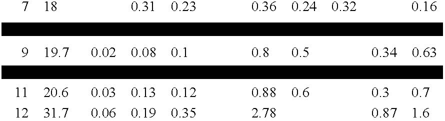

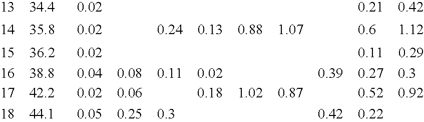

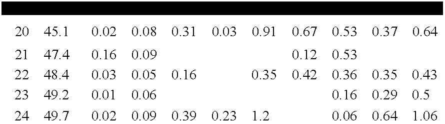

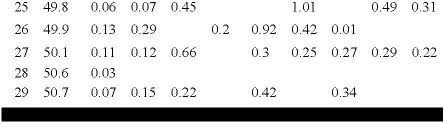

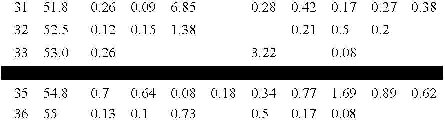

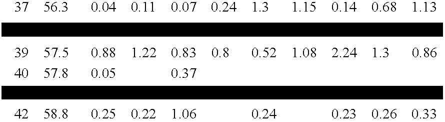

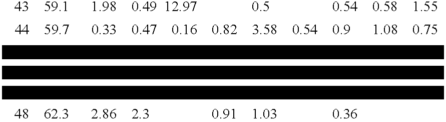

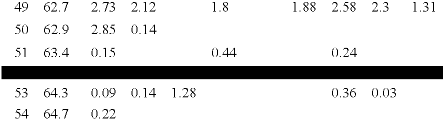

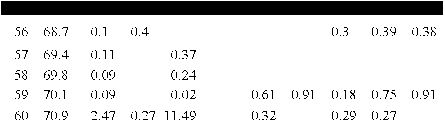

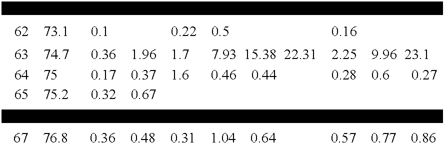

| | | | | | | | | | |
|---|---|---|---|---|---|---|---|---|---|
| fatty | 71.41 | 17.81 | 17.54 | 55.76 | 9.77 | 8.8 | 18.32 | 22.62 | 10.08 |
| acid | | | | | | | | | |
| C16 + | 70.55 | 17.08 | 17.46 | 55.58 | 9.43 | 7.91 | 16.1 | 21.73 | 9.46 |
| C18 | | | | | | | | | |
| ester | 5.14 | 18.3 | 12.21 | 13.69 | 25.11 | 33.39 | 7.15 | 17.76 | 36.06 |
| alcohol | 16.46 | 26.63 | 16.42 | 24.27 | 19.13 | 27.43 | 50.67 | 36.07 | 25.74 |
| hydro- | 5.66 | 36.16 | 46.14 | 0.48 | 5.83 | 4.21 | 2.04 | 3.85 | 5.32 |
| carbon | | | | | | | | | |
| alde- | 0.67 | 1.48 | 0.69 | 3.5 | 17.84 | 12.15 | 9.13 | 5.14 | 8.2 |
| hyde | | | | | | | | | |
| total | 99.34 | 98.9 | 92.31 | 97.7 | 77.68 | 85.98 | 87.31 | 85.44 | 85.4 |

TABLE 10
Elderberry essential oil compounds identified by GC-MS.

| Peak # | Ret time (min) | Compound name | CAS # | Formula |
|---|---|---|---|---|
| 1 | 7.1 | 2-heptenal, (E)- | 18829-55-5 | C7H12O |
| 2 | 7.2 | 2-Heptenal, (Z)- | 57266-86-1 | C7H12O |
| 3 | 8.4 | 2,4-heptadienal, (E,E)- | 4313-03-5 | C7H10O |
| 4 | 12.1 | nonanal | 124-19-6 | C9H18O |
| 5 | 17.5 | 1,3-bis(1,1-dimethylethyl)benzene | 1014-60-4 | C14H22 |
| 6 | 17.7 | 2-dodecenal | 20407-84-5 | C12H22O |
| 7 | 18.0 | 3-phenyl-2-propenal | 104-55-2 | C9H8O |
| 8 | 19.0 | 2,4-decanienal | 2363-88-4 | C10H16O |
| 9 | 19.7 | 8-methyl-1-undecene | 74630-40-3 | C12H24 |
| 10 | 20.1 | 2,4-decanienal, (E,E)- | 25152-84-5 | C10H16O |
| 11 | 20.6 | hexyl octyl ether | 17071-54-5 | C14H30O |
| 12 | 31.7 | 1-undecanol | 112-42-5 | C11H24O |
| 13 | 34.4 | 2,3,3-trimethyl-octane | 62016-30-2 | C11H24 |
| 14 | 35.8 | Unknown 1 | | |
| 15 | 36.2 | β-Farnesene | 18794-84-8 | C15H24 |
| 16 | 38.8 | Unknown 3 | | |
| 17 | 42.2 | 2-dodecanol, 2-methyl- | 1653-37-8 | C13H28O |
| 18 | 44.1 | 1-dodecanol | 112-53-8 | C12H26O |
| 19 | 44.8 | 2-propenoic acid, tridecyl ester | 3076-04-8 | C16H30O2 |
| 20 | 45.1 | 3.7-dimethyl-undecane | 17301-29-0 | C13H28 |
| 21 | 47.4 | tetradecanoic acid | 544-63-8 | C14H28O2 |
| 22 | 48.4 | Unknown 2 | | |
| 23 | 49.2 | Tetradecanal | 124-25-4 | C14H28O |
| 24 | 49.7 | 1,8-nonanediol, 8-methyl- | 54725-73-4 | C10H22O2 |
| 25 | 49.8 | caffeine | 58-08-2 | C8H10N4O2 |
| 26 | 49.9 | pentadecanal | 2765-11-9 | C15H30O |
| 27 | 50.1 | 2-Pentadecanone, 6,10,14-trimethyl- | 502-69-2 | C18H36O |
| 28 | 50.6 | octadecanoic acid | 57-11-4 | C18H36O2 |
| 29 | 50.7 | Unknown 3 | | |
| 30 | 51.2 | 1-Hexadecanol | 36653-82-4 | C16H34O |
| 31 | 51.8 | octadecane | 593-45-3 | C18H38 |
| 32 | 52.5 | Hexadecanoic acid, methyl ester | 112-39-0 | C17H34O2 |
| 33 | 53.0 | 9-Octadecenoic acid (Z)- | 112-80-1 | C18H34O2 |
| 34 | 54.1 | n-hexadecanoic acid | 57-10-3 | C16H32O2 |
| 35 | 54.8 | hexadecanoic acid, ethyl ester | 628-97-9 | C18H36O2 |
| 36 | 55.0 | Nonadecane | 629-92-5 | C19H40 |
| 37 | 56.3 | Unknown 5 | | |
| 38 | 57.2 | 9-Octadecen-1-ol, (Z)- | 143-28-2 | C18H36O |
| 39 | 57.5 | 9-Octadecen-1-ol, (E)- | 506-42-3 | C18H36O |
| 40 | 57.8 | Unknown 6 | | |
| 41 | 58.3 | 1-octadecanol | 112-92-5 | C18H38O |
| 42 | 58.8 | 9,12-Octadecadienoic acid, methyl ester, (E,E)- | 2566-97-4 | C19H34O2 |
| 43 | 59.1 | eicosane | 112-95-8 | C20H42 |
| 44 | 59.7 | phytol | 150-86-7 | C20H40O |
| 45 | 60.7 | Stearolic acid | 506-24-1 | C18H32O2 |
| 46 | 61.6 | 9,12-Octadecadienoic acid(Z,Z)- | 60-33-3 | C18H32O2 |
| 47 | 62.0 | Linoelaidic acid | 506-21-8 | C18H32O2 |
| 48 | 62.3 | 9,12-Octadecadienoic acid, methyl ester, (E,E)- | 2642-85-3 | C19H34O2 |
| 49 | 62.7 | 9,12,15-octadecatrien-1-ol | 506-44-5 | C18H32O |
| 50 | 62.9 | Octadecanoic acid | 57-11-4 | C18H36O2 |
| 51 | 63.4 | Unknown 7 | | |
| 52 | 63.8 | hexadecanoic acid, butyl ester | 111-06-8 | C20H40O2 |
| 53 | 64.3 | octadecanoic acid, ethyl ester | 111-61-5 | C20H40O2 |
| 54 | 64.7 | heneicosane | 629-94-7 | C21H44 |
| 55 | 67.1 | squalene | 7683-64-9 | C30H50 |
| 56 | 68.7 | Unknown 8 | | |
| 57 | 69.4 | 1-nonadecene | 18435-45-5 | C19H38 |
| 58 | 69.8 | 10-heneicosene | 95008-11-0 | C21H42 |
| 59 | 70.1 | 1-Eicosanol | 629-96-9 | C20H42O |
| 60 | 70.9 | Docosane | 629-97-0 | C22H46 |
| 61 | 71.9 | Hexadecyl pentanoate | 125164-54-7 | C21H42O2 |
| 62 | 73.1 | 4,8,12,16-tetramethylheptadecan-4-olide | 96168-15-9 | C21H40O2 |
| 63 | 74.7 | octadecanoic acid, butyl ester | 123-95-5 | C22H44O2 |
| 64 | 75.0 | Eicosanoic acid, butyl ester | 18281-05-5 | C22H44O2 |
| 65 | 75.2 | 8-heptyl-pentadecane | 71005-15-7 | C22H46 |
| 66 | 76.4 | 1-tricosene | 18835-32-0 | C23H46 |
| 67 | 76.8 | lauric acid, 2-butoxyethyl ester | 109-37-5 | C18H36O3 |

| Peak # | Mw | structure | category |
|---|---|---|---|
| 1 | 112 | 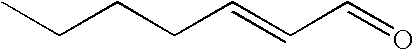 | C7 aldehyde |
| 2 | 112 | 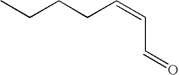 | C7 aldehyde |
| 3 | 110 | 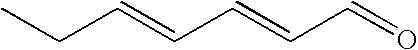 | C7 aldehyde |
| 4 | 142 | 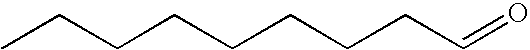 | C9 aldehyde |
| 5 | 190 | 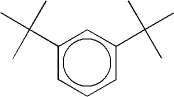 | aromatic |
| 6 | 182 | 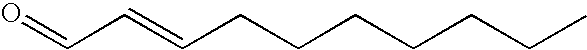 | C12 aldehyde |
| 7 | 132 | 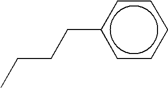 | aromatic |
| 8 | 152 | 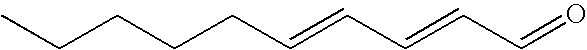 | C10 aldehyde |
| 9 | 168 | 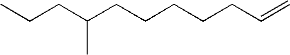 | C12 alkene |
| 10 | 152 | 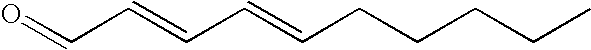 | C10 aldehyde |
| 11 | 214 | 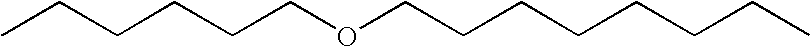 | ethaer |
| 12 | 172 | 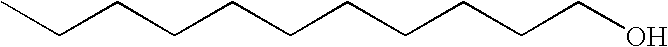 | C11 alcohol |
| 13 | 156 | 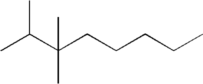 | C11 alkane |
| 14 | | | |
| 15 | 204 | 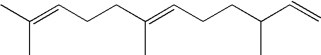 | C15 alkene |
| 16 | | | |
| 17 | 200 | 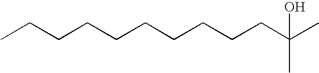 | C13 alcohol |
| 18 | 186 | 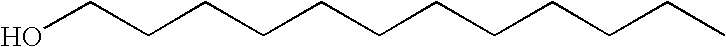 | C12 alcohol |
| 19 | 254 | 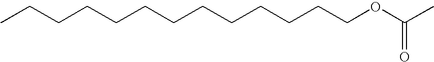 | C13 alcohol ester |
| 20 | 184 | 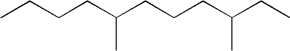 | C13 alkane |
| 21 | 228 | 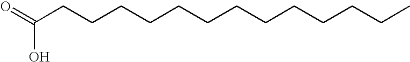 | C14 acid |
| 22 | | | |
| 23 | 212 | 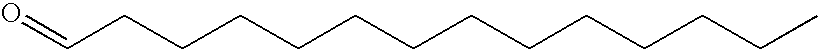 | C14 aldehyde |
| 24 | 174 | 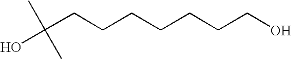 | C10 alcohol |
| 25 | 194 | 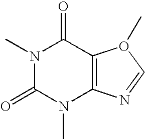 | |
| 26 | 226 | 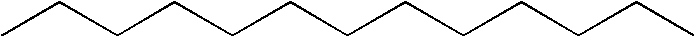 | C15 aldehyde |
| 27 | 268 | 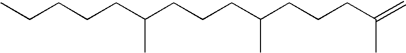 | C18 ketone |
| 28 | 284 | 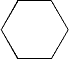 | C18 acid |
| 29 | | | |
| 30 | 242 | 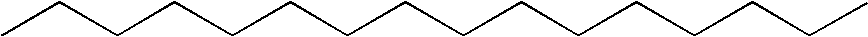 | C16 alcohol |
| 31 | 254 | 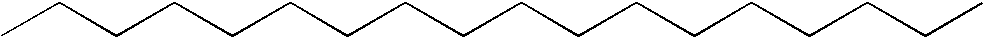 | C18 alkane |
| 32 | 270 | 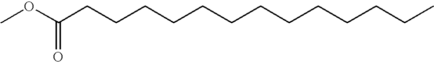 | C16 acid ester |
| 33 | 282 | 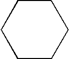 | C18 acid |
| 34 | 256 | 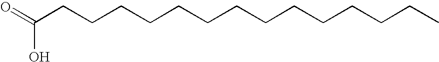 | C16 acid |
| 35 | 284 | 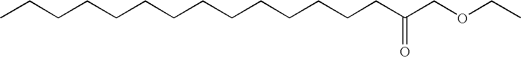 | C16 acid ester |
| 36 | 268 | 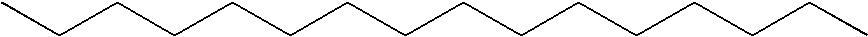 | C19 alkane |
| 37 | | | |
| 38 | 268 | 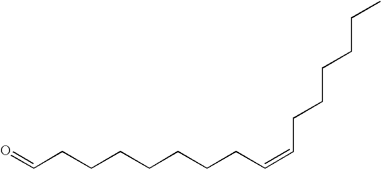 | C18 alcohol |
| 39 | 268 | 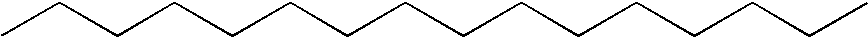 | C18 alcohol |
| 40 | | | |
| 41 | 270 | 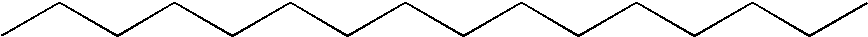 | C18 alcohol |
| 42 | 294 | 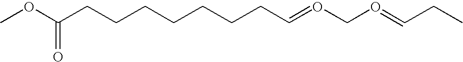 | C18 acid ester |
| 43 | 282 | 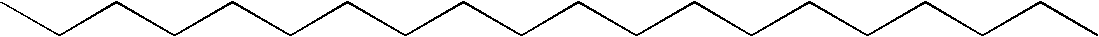 | C20 alkene |
| 44 | 296 | 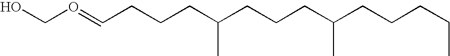 | |
| 45 | 280 | 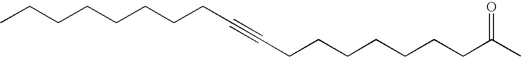 | |
| 46 | 280 | 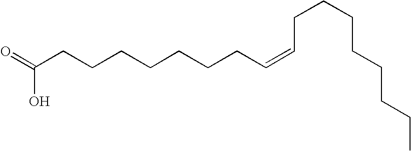 | C18 acid |
| 47 | 280 | 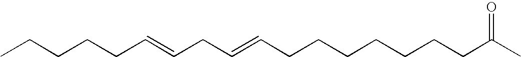 | C18 acid |
| 48 | 294 | 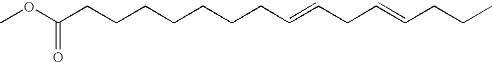 | C18 acid ester |
| 49 | 264 | 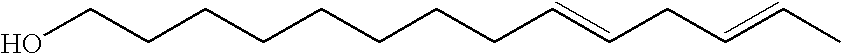 | C18 alcohol |
| 50 | 284 | 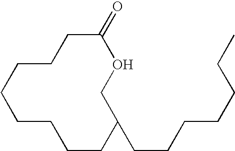 | C18 acid |
| 51 | | | |
| 52 | 312 | 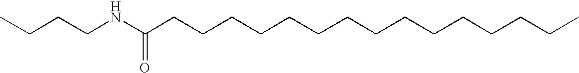 | C16 acid ester |
| 53 | 312 | 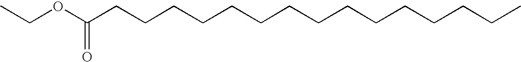 | C18 ester |
| 54 | 296 | 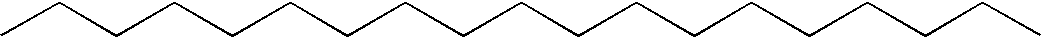 | C21 alkane |
| 55 | 410 | 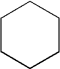 | |
| 56 | | | |
| 57 | 266 | 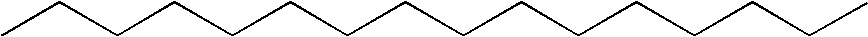 | C19 alkene |
| 58 | 294 | 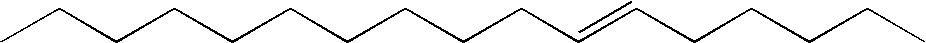 | C21 alkene |
| 59 | 298 | 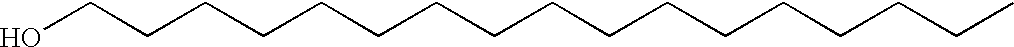 | C20 alcohol |
| 60 | 310 | 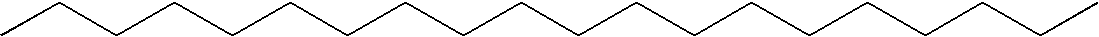 | C22 alkane |
| 61 | 326 | 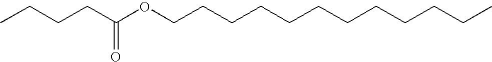 | ester |
| 62 | 324 | 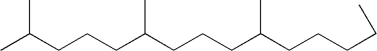 | ester |
| 63 | 340 | 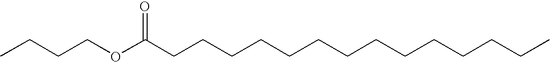 | Ester |
| 64 | 340 | 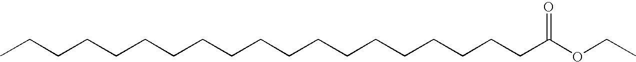 | Ester |
| 65 | 310 | 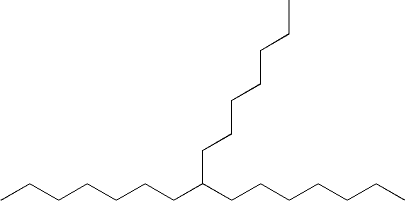 | C22 alkane |
| 66 | 322 | 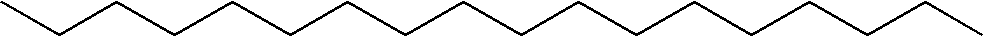 | C23 alkene |
| 67 | 300 | 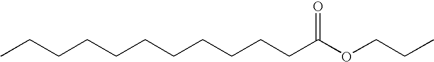 | ester |

These results demonstrate the effect of pressure on the kinetics of extraction.

Higher extraction pressures result in the system reaching equilibrium at shorter times with less amount of CO₂ consumed. The total extraction yield increases with increasing extraction pressure due to the density increase associated with pressure increase. Interestingly, a lower pressures such as 100-300 bar, the lower the temperature, the higher the yield again related to a higher density. At higher pressures such as 300-500 bar, temperature has far less effect of the extraction yield. Although a higher yield and greater efficiency of extraction may be achieved with pressures greater than 300 bar, 95% purity of the essential oil chemical constituents can be achieved with pressures less than 300 bar and temperatures of about 40-60° C.

In the experiment range investigated, it can be clearly noted that there is a competition effect between temperature and density. This aspect is well defined and documented in the literature, where an increase in pressure, at constant temperature, leads to an increase in the yield due to the enhancement in the solvency power of the supercritical and near critical fluid. An increase in temperature promotes an enhancement in vapor pressure of the compounds favoring the extraction. Additionally, the increase in diffusion coefficient and the decrease in solvent viscosity also help the compounds extraction from the herbaceous porous matrix as the temperature is increased to higher value. On the other hand, an increase in temperature, at constant system pressure, leads to a decrease in the solvent density.

Sixty-seven compounds were separated and identified in elderberry essential oil using GC-MS analysis according to the mass spectrum of each compound (Tables 9 and 10). The compounds varied from 7 carbon compounds (C7) to 23 carbon compounds (C23) including: 9 aldehydes (C7-C15) having retention times of 7-50 min, the principal ones being the unsaturated C7 and C10 aldehydes (compounds #1, 2, 6, &8 of Table 5); 111 alcohols (C13-C20); 12 esters (C13-C22); 7 fatty acids (C14-C22); and other aromatic and aliphatic compounds. Based on known bioactivity, the most important compounds appear to be the C16 and C18 saturated and unsaturated fatty acid, alcohol, and its ester. For example, hexadecanol (#30), hexadecanoic acid (#34), hexadecanoic acid methyl ester (#32), hexadecanoic acid ethyl ester (#35), and hexadecanoic acid butyl ester (#52) all belong to the C16 compounds. Saturated octadecanoic acid and its esters octadecanoic acid ethyl ester (#53) and octadecanoic acid butyl ester, mono-unsaturated fatty acids 9-octadecen-1-ol isomers (#38, 39), poly-unsaturated fatty acids 9,12-octaecanienoic acid isomers (#46, 48) belong to the C18 compounds. The common names of C16 and C18 fatty acids are called palmatic acid and stearic acid.

In Table 9, the highlighted compounds are the higher concentration compounds found in the essential oil fractions. It should be noted that the ratios of the compounds vary with different SCCO₂ extraction conditions. For example, at low pressures such as 100 bar, C16 and C18 fatty acids are in higher concentration with a low total extraction yield. In contrast, C16 and C18 fatty acid esters are found in higher concentration at high extraction temperatures.

Interestingly, squalene is extracted in high concentrations of about 23% in the 40° C. and 300 bar essential oil fractions and lower concentration of about 8% in the 40° C. and 500 bar fraction. Squalene has been investigated as an adjunctive therapy for some human cancers. In animal models it has proved to be effective in inhibiting lung cancer. It has also been shown to have chemopreventive effects against colon cancer in animal models. Supplementation with squalene in animal models has been shown to enhance immune function and reduce cholesterol levels.

In conclusion, the concentration of certain elder species essential oil chemical constituents can be altered using different SFE conditions. Such differential SFE extraction properties can be used to further enhance or decrease the concentration of certain compounds in purified essential oil sub-fractions by using sequential multi-stage SCCO₂ fractionation as illustrated in Step 1B, FIG. 1 or a multi-collector fractionation system.

Step 2. Hydroalcoholic Leaching Process for Extraction of Crude Phenolic Acid Fraction

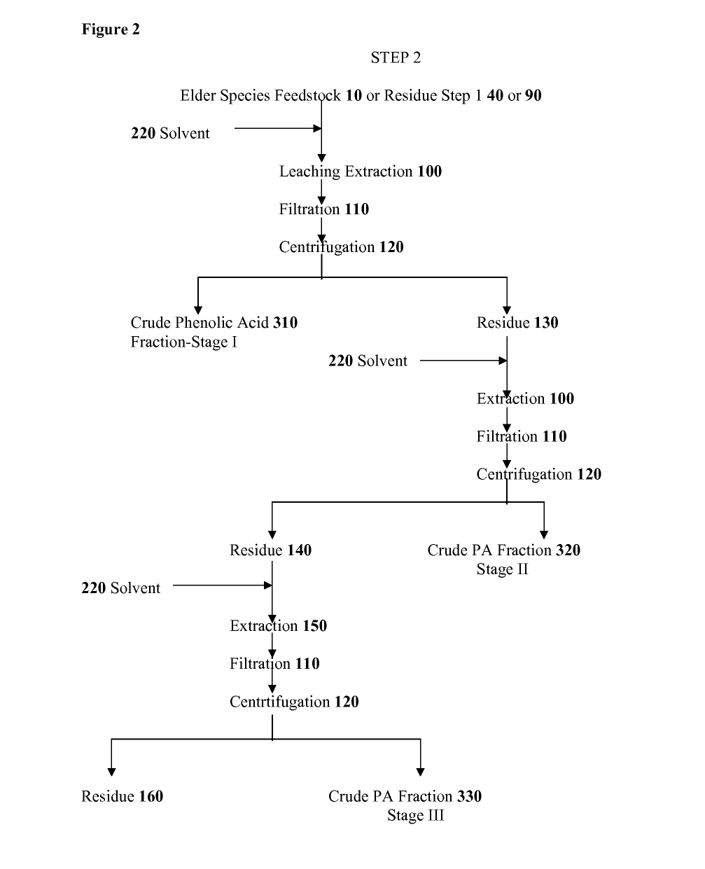

In one aspect, the present invention comprises extraction and concentration of the bio-active phenolic acid chemical constituents while preserving the lectins and polysaccharides in the residue for separate extraction and purification (Step 4). A generalized description of this step is diagrammed in FIG. 2. This Step 2 extraction process is a solvent leaching process. The feedstock for this extraction is either elder species ground dry plant material 10 or the residue 40 from the Step 1 SCCO₂ extraction of the essential oil chemical constituents. The extraction solvent 220 is aqueous ethanol. The extraction solvent may be 10-95% aqueous alcohol, 80% aqueous ethanol is preferred. In this method, the elder feedstock material and the extraction solvent are loaded into an extraction vessel 100, 150 that is heated and stirred. It may be heated to 100° C., to about 90° C., to about 80° C., to about 70° C., or to about 60-90° C. The extraction is carried out for about 1-10 hours, for about 1-5 hours, for about 2 hours. The resultant fluid-extract is filtered 110 and centrifuged 120. The filtrate (supernatant) 310, 320, 330 is collected as product, measured for volume and solid content dry mass after evaporation of the solvent. The extraction residue material 160 is retained and saved for further processing (see Step 4). The extraction may be repeated as many times as is necessary or desired. It may be repeated 1 or more times, 2 or more times, 3 or more times, etc. For example, FIG. 2 shows a three-stage process, where the second stage and the third stage use the same methods and conditions. An example of this extraction step is found in Example 2. The results are shown in Table 11.

TABLE 11
Leaching extraction crude phenolic acid yield and purity of elderberry.

| | | | | | | Yield (%) | | | |
|---|---|---|---|---|---|---|---|---|---|
| | | Purity (%) | | | | Total | | | |
| | Yield | Total phenolic | | | | phenolic | | | |
| | (%) | acids | Total* anthocyanidin | CY3glu | Rutin | acids | Total anthocyanidin | CY3glu | Rutin |
| Elderberry | 35.6 | 4.34 | 0.178 | 0.107 | 0.762 | 1.55 | 0.06 | 0.04 | 0.27 |

The total crude phenolic acid extraction yield was about 35% by mass weight of the original native elderberry feedstock with a total phenolic acid extraction yield of 1.6% and phenolic acid purity of 4.3% by mass weight of the fraction. The anthocyanidin extraction yield in the crude phenolic acid fraction was 0.06% by mass weight of the original elderberry feedstock with a purity (concentration) of 0.18 by mass weight of the fraction. The principal phenolic acid was rutin and the principal anthocyanidin was cyaniding-3-glucoside. These data are all consistent with the literature. This crude phenolic acid composition may be used either as a final product or as a feedstock for further processing to purify the desirable phenolic acid chemical constituents (Step 3).

Step 3. Affinity Adsorbent Extraction Process

As taught herein, a purified phenolic acid fraction extract from elder and related species may be obtained by contacting a hydroalcoholic extract of elder feedstock with a solid affinity polymer adsorbent resin so as to adsorb the active phenolic acids contained in the hydroalcolholic extract onto the affinity adsorbent. The bound chemical constituents are subsequently eluted by the methods taught herein. Prior to eluting the phenolic acid fraction chemical constituents, the affinity adsorbent with the desired chemical constituents adsorbed thereon may be separated from the remainder of the extract in any convenient manner, preferably, the process of contacting with the adsorbent and the separation is effected by passing the aqueous extract through an extraction column or bed of the adsorbent material.

A variety of affinity adsorbents can be utilized to purify the phenolic acid chemical constituents of elder species, such as, but not limited to “Amberlite XAD-2” (Rohm & Hass), “Duolite S-30” (Diamond Alkai Co.), “SP207” (Mitsubishi Chemical), ADS-5 (Nankai University, Tianjin, China), ADS-17 (Nankai University, Tianjin, China), Dialon HP 20 (Mitsubishi, Japan), and Amberlite XAD7 HP (Rohm & Hass). Amberlite XAD7 HP is preferably used due to the high affinity for the phenolic acid chemical constituents of elder and related species.

Although various eluants may be employed to recover the phenolic acid chemical constituents from the adsorbent, in one aspect of the present invention, the eluant comprises low molecular weight alcohols, including, but not limited to, methanol, ethanol, or propanol. In a second aspect, the eluant comprises low molecular alcohol in an admixture with water. In another aspect, the eluant comprises low molecular weight alcohol, a second organic solvent, and water.

Preferably, the elder species feedstock has undergone a one or more preliminary purification process such as, but not limited to, the processes described in Step 1 and 2 prior to contacting the aqueous phenolic acid chemical constituent containing extract with the affinity adsorbent material.

Using affinity adsorbents as taught in the present invention results in highly purified phenolic acid chemical constituents of the elder species that are remarkably free of other chemical constituents which are normally present in natural plant material or in available commercial extraction products. For example, the processes taught in the present invention can result in purified phenolic acid extracts that contain total phenolic acid chemical constituents in excess of 40% and total anthocyanidins in excess of 2% by dry mass weight.

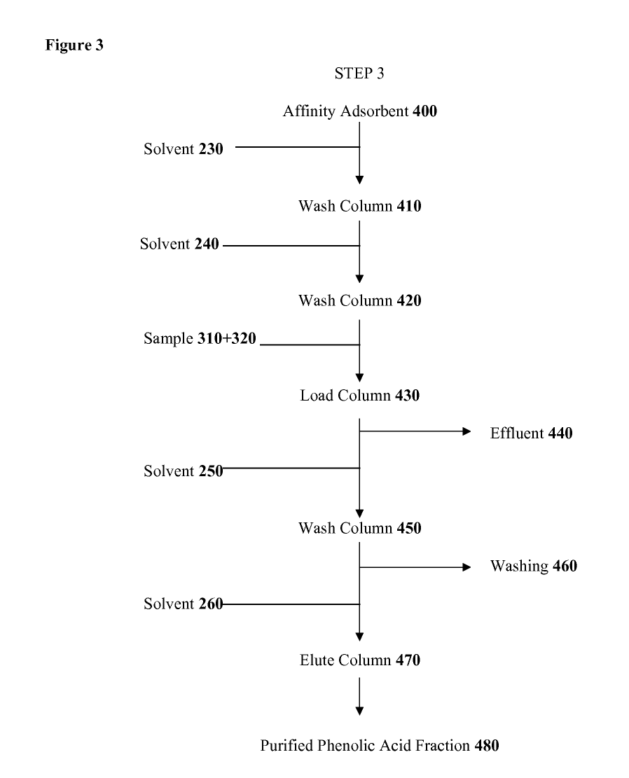

A generalized description of the extraction and purification of the phenolic acids from the leaves of the elder species using polymer affinity adsorbent resin beads is diagrammed in FIG. 3. The feedstock for this extraction process may be the aqueous ethanol solution containing the phenolic acids from Step 2 Water Leaching Extraction 310+/−320+/−330. The appropriate weight of adsorbent resin beads (5 mg of phenolic acids per gm of adsorbent resin) is washed with 4-5 BV ethanol 230 and 4-5 BV distilled water 240 before and after being loaded into a column 410, 420. The phenolic acid containing aqueous solution 310+320 is then loaded onto the column 430 at a flow rate of 3 to 5 bed volume (BV)/hour. Once the column is fully loaded, the column is washed 450 with distilled water 250 at a flow rate of 2-3 BV/hour to remove any impurities from the adsorbed phenolic acids. The effluent residue 440 and washing residue 460 were collected, measured for mass content, phenolic acid content, and discarded. Elution of the adsorbed phenolic acids 470 is accomplished in an isocratic fashion with 40 or 80% ethanol/water as an eluting solution 260 at a flow rate of 3-4 BV/hour and the elution curve was recorded for the eluant extract (extracts) 480. Elution volumes 480 may be collected about every 25 minutes and these samples are analyzed using HPLC and tested for solids content and purity. An example of this extraction process is found in Example 3. The results are shown in Tables 12 and 13.

TABLE 12
Mass balance and HPLC analysis results on different fractions eluted from XAD
7HP column.

| | Purity (%) | | | | Weight of each compound (mg) | | | | |
|---|---|---|---|---|---|---|---|---|---|
| | Total | | | | Total | | | | Total |
| | phenolic | Total | CY3 | | phenolic | Total | CY3 | | solid |
| Sample | acids | anthocyanidin | glu | Rutin | acids | anthocyanidin | glu | Rutin | (g) |
| XAD | 5.39 | 0.24 | 0.12 | 0.75 | 78.27 | 4.02 | 1.95 | 12.55 | 1.45 |
| 7HP | | | | | | | | | |
| loading | | | | | | | | | |
| effluent | 0.00 | 0.01 | 0.00 | 0.00 | 0.00 | 0.04 | 0.00 | 0.00 | 0.73 |
| washing | 0.00 | 0.06 | 0.00 | 0.00 | 0.00 | 0.06 | 0.00 | 0.00 | 0.61 |
| F1 | 3.91 | 0.01 | 0.00 | 0.00 | 0.78 | 0.09 | 0.00 | 0.00 | 0.02 |
| (20 ml) | | | | | | | | | |
| F2 | 27.81 | 2.43 | 1.01 | 0.23 | 12.12 | 1.06 | 0.44 | 0.10 | 0.05 |
| (20 ml) | | | | | | | | | |
| F3 | 31.02 | 2.99 | 1.67 | 2.60 | 9.38 | 0.90 | 0.51 | 0.78 | 0.03 |
| (18 ml) | | | | | | | | | |
| F4 | 40.49 | 2.92 | 1.91 | 5.74 | 4.26 | 0.31 | 0.20 | 0.60 | 0.01 |
| (10 ml) | | | | | | | | | |
| F5 | 31.87 | 1.29 | 1.01 | 16.28 | 13.47 | 0.55 | 0.43 | 6.88 | 0.04 |
| (17 ml) | | | | | | | | | |
| F6 | 36.78 | 0.80 | 0.59 | 17.01 | 8.30 | 0.18 | 0.13 | 3.84 | 0.02 |
| (27 ml) | | | | | | | | | |
| F2-F6 | 28.4 | 1.8 | 1.0 | 7.2 | 48.31 | 3.09 | 1.71 | 12.2 | 0.17 |

TABLE 13
Mass balance and HPLC analysis results on different fractions eluted from ADS5
column.

| | Purity (%) | | | | Weight of each compound (mg) | | | | |
|---|---|---|---|---|---|---|---|---|---|
| | Total | | | | Total | | | | Total |
| | phenolic | Total | CY3 | | phenolic | Total | CY3 | | solid |
| Sample | acids | anthocyanidin | glu | Rutin | acids | anthocyanidin | glu | Rutin | (g) |
| ADS5 | 6.2 | 0.19 | 0.09 | 0.76 | 87.21 | 2.65 | 1.26 | 10.67 | 1.41 |
| loading | | | | | | | | | |
| effluent | 0 | 0.00 | 0.00 | 0.00 | 0.00 | 0.00 | 0.00 | 0.00 | 0.73 |
| washing | 0 | 0.00 | 0.00 | 0.00 | 0.00 | 0.00 | 0.00 | 0.00 | 0.49 |
| F1 | 0.0 | 0.00 | 0.00 | 0.00 | 0.00 | 0.00 | 0.00 | 0.00 | 0.00 |
| (20 ml) | | | | | | | | | |
| F2 | 41.8 | 2.20 | 1.01 | 0.41 | 57.13 | 3.01 | 1.46 | 0.56 | 0.14 |
| (20 ml) | | | | | | | | | |
| F3 | 34.3 | 0.04 | 0.03 | 29.12 | 7.04 | 0.01 | 0.005 | 5.98 | 0.02 |
| (17 ml) | | | | | | | | | |
| F4 | 20.7 | 0.003 | 0 | 11.20 | 7.17 | 0.005 | 0.005 | 3.89 | 0.03 |
| (17 ml) | | | | | | | | | |
| F3-F4 | 28.42 | 0.03 | 0.02 | 19.74 | 14.21 | 0.015 | 0.01 | 9.87 | 0.05 |

As taught herein, the affinity adsorbents XAD7HP and ADS5 can further purify (concentrate) the flavanoid and anthocyanidin phenolic acids of elder species plant material. The purity of total phenolic acids of greater than 40%, total anthocyanidins of greater than 2.8%, and rutin of greater than 29% by mass weight of the respective eluate sub-fraction. These represent a greater 10-fold increase in concentration over than that found in elder species native plant material or known and greater than 5-fold increase in concentration over that found in available elder species extraction products. Greater than 60% yield by mass weight of the phenolic acid chemical constituents of the loading solutions are retrieved in the eluant. Based on the original elder feedstock, to total phenolic acid yield is about 4.2% by mass weight of the original feedstock material. In fact, almost no rutin or anthocyanidins could be detected in the effluent or washing solutions. Interestingly, ADS5 has a rather unique advantage in that it is possible to separate the anthocyanidins from rutin in different sub-fractions by using the different concentrations of ethanol solutions. For example, the ADS5 40% ethanol elution fraction (F2) concentrates the anthocyanidins greater than 10-fold whereas the combined sub-fractions (F3+F4) concentrates rutin greater than 25-fold with little or no concentration of the anthocyanidins. Therefore, the Step 3 affinity adsorbent process can yield novel purified phenolic acid sub-fractions with novel chemical constituent profiles.

Step 4. Lectin-Polysaccharide Fraction Extraction Processes

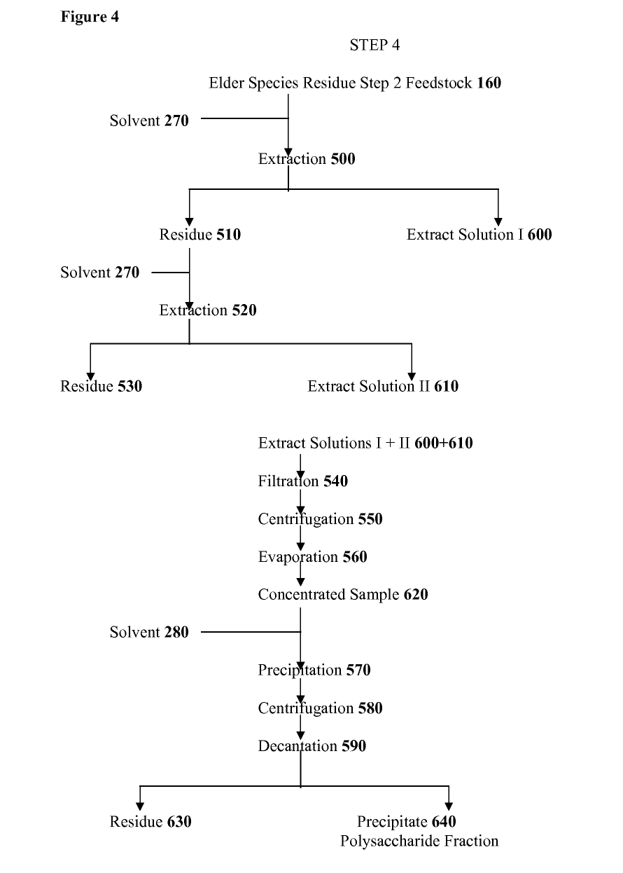

The lectin-polysaccharide extract fraction of the chemical constituents of elder species has been defined in the scientific literature as the “water soluble, ethanol insoluble extraction fraction”. A generalized description of the extraction of the polysaccharide fraction from extracts of elder species using water solvent leaching and ethanol precipitation processes is diagrammed in FIG. 4. The feedstock 160 is the solid residue from the hydroalcoholic leaching extraction process of Step 2. This feedstock is leaching extracted in two stages. The solvent is distilled water 270. In this method, the elder species residue 160 and the extraction solvent 270 are loaded into an extraction vessel 500, 520 and heated and stirred. It may be heated to 100° C., to about 80° C., or to about 70-90° C. The extraction is carried out for about 1-5 hours, for about 2-4 hours, or for about 2 hours. The two stage extraction solutions 600+610 are combined and the slurry is filtered 540, centrifuged 550, and evaporated 560 to remove water until an about 8-fold increase in concentration of the chemicals in solution 620. Anhydrous ethanol 280 is then used to reconstitute the original volume of solution making the final ethanol concentration at 60-80%. A large precipitate 570 is observed. The solution is centrifuged 580, decanted 590 and the supernatant residue 730 is discarded. The precipitate product 640 is the purified lectin-polysaccharide fraction that may be analyzed for polysaccharides using the colormetric method by using Dextran 5,000-410,000 molecular weight as reference standards and for protein using Bradford protein analysis method. The purity of the extracted polysaccharide fraction is about 100-170 mg/g dextran standard equivalents with a total yield of 2.4-3.5% by % mass weight of the original native elder plant material feedstock. The purity of the extracted lectin proteins is about 16% by mass weight of the lectin-polysaccharide fraction with a total yield of 0.56% by % mass weight of the original native elder plant material. An example of this process is given in Example 4. The results are shown in Tables 14 and 15. Moreover, AccuTOF-DART mass spectrometry (see Exemplification section) was used to further profile the molecular weights of the compounds comprising the purified polysaccharide fraction.

TABLE 14
Polysaccharide analysis of elderberry lectin-polysaccharide fractions

| | | Elderberry |
|---|---|---|
| Total yield (%) | | 10.5 |
| 60% precipitate yield (%) | | 2.43 |
| 80% precipitate yield (%) | | 3.45 |
| 60% | Dextran 5K (g/g pcp) | 0.15 |
| precipitate | Dextran 50K (g/g pcp) | 0.16 |
| | Dextran 410K (g/g pcp) | 0.10 |
| 80% | Dextran 5K (g/g pcp) | 0.16 |
| precipitate | Dextran 50K (g/g pcp) | 0.17 |
| | Dextran 410K (g/g pcp) | 0.14 |

TABLE 15
Protein analysis of elderberry lectin-polysaccharide fractions.

| | Purity of | Yield of |
|---|---|---|
| sample | protein (%) | protein (%) |
| Elderberry water crude extracts | 5.63 | 0.59 |
| 60% precipitates from | 4.81 | 0.12 |
| Elderberry | | |
| 80% precipitates from | 16.17 | 0.56 |
| Elderberry | | |

The total elder lectin-polysaccharide yield was 2.43% with 60% ethanol precipitation and 3.45% with 80% ethanol precipitation by % mass weight based on the original native elderberry feedstock material. Based on multiple experiments with elder species plant material as well as other botanicals and the scientific literature, it would appear that the 3.5% yield of the lectin-polysaccharide fraction is very close to the concentration of water soluble-ethanol insoluble polysaccharide and lectin proteins present in the raw elder species plant material.

The purity of the polysaccharides was from 100 to 170 mg/gm of dextran equivalents. Although the dextran equivalents of the polysaccharide fractions appear somewhat lower than that found with purified polysaccharide fractions from other botanicals, the molecular weights of the polysaccharides in elder species plant material are not known. Hence, the purity of the polysaccharide chemical constituents may be much greater in the elder species purified polysaccharide fraction than that estimated using the colormetic assay with dextran equivalents.

The purity of the lectin protein in the elder lectin-polysaccharide fractions was 4.8% with 60% ethanol precipitation and 16.2% with 80% ethanol precipitation by % mass weight of the fraction. The total lectin protein yield with 80% ethanol precipitation was 0.56% by mass weight based on the original native elder species feedstock and about 95% by mass weight based on the crude water leaching extract. The total lectin yield with 60% ethanol precipitation is only about 20% by mass weight based on the crude water leaching extract. The 60% ethanol precipitation results in a higher purity of polysaccharide chemical constituents and lower purity of lectin proteins. Therefore, using the two-stage ethanol precipitation, it is possible to have a high polysaccharide concentration low lectin protein concentration profile (˜0/1) sub-fraction using 60% ethanol followed by a second stage precipitation using 80% ethanol to yield a low polysaccharide/high lectin protein concentration profile (˜2/1) sub-fraction.

Many methods are known in the art for removal of alcohol from solution. If it is desired to keep the alcohol for recycling, the alcohol can be removed from the solutions, after extraction, by distillation under normal or reduced atmospheric pressures. The alcohol can be reused. Furthermore, there are also many methods known in the art for removal of water from solutions, either aqueous solutions or solutions from which alcohol was removed. Such methods include, but not limited to, spray drying the aqueous solutions onto a suitable carrier such as, but not limited to, magnesium carbonate or maltodextrin, or alternatively, the liquid can be taken to dryness by freeze drying or refractive window drying.

Food and Medicaments

As a form of foods of the present invention, there may be formulated to any optional forms, for example, a granule state, a grain state, a paste state, a gel state, a solid state, or a liquid state. In these forms, various kinds of substances conventionally known for those skilled in the art which have been allowed to add to foods, for example, a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller, etc. may be optionally contained. An amount of the elderberry extract to be added to foods is not specifically limited, and for example, it may be about 10 mg to 5 g, preferably 50 mg to 2 g per day as an amount of take-in by an adult weighing about 60 kg.

In particular, when it is utilized as foods for preservation of health, functional foods, etc., it is preferred to contain the effective ingredient of the present invention in such an amount that the predetermined effects of the present invention are shown sufficiently.

The medicaments of the present invention can be optionally prepared according to the conventionally known methods, for example, as a solid agent such as a tablet, a granule, powder, a capsule, etc., or as a liquid agent such as an injection, etc. To these medicaments, there may be formulated any materials generally used, for example, such as a binder, a disintegrant, a thickener, a dispersant, a reabsorption promoting agent, a tasting agent, a buffer, a surfactant, a dissolution aid, a preservative, an emulsifier, an isotonicity agent, a stabilizer or a pH controller.

An administration amount of the effective ingredient (elderberry extract) in the medicaments may vary depending on a kind, an agent form, an age, a body weight or a symptom to be applied of a patient, and the like, for example, when it is administrated orally, it is administered one or several times per day for an adult weighing about 60 kg, and administered in an amount of about 10 mg to 5 g, preferably about 50 mg to 2 g per day. The effective ingredient may be one or several components of the elder extract.

Delivery Systems

Administration modes useful for the delivery of the compositions of the present invention to a subject include administration modes commonly known to one of ordinary skill in the art, such as, for example, powders, sprays, ointments, pastes, creams, lotions, gels, solutions, patches and inhalants.

In one embodiment, the administration mode is an inhalant which may include timed-release or controlled release inhalant forms, such as, for example, liposomal formulations. Such a delivery system would be useful for treating a subject for SARS, bird flu, and the like. In this embodiment, the formulations of the present invention may be used in any dosage dispensing device adapted for intranasal administration. The device should be constructed with a view to ascertaining optimum metering accuracy and compatibility of its constructive elements, such as container, valve and actuator with the nasal formulation and could be based on a mechanical pump system, e.g., that of a metered-dose nebulizer, dry powder inhaler, soft mist inhaler, or a nebulizer. Due to the large administered dose, preferred devices include jet nebulizers (e.g., PARI LC Star, AKITA), soft mist inhalers (e.g., PARI e-Flow), and capsule-based dry powder inhalers (e.g., PH&T Turbospin). Suitable propellants may be selected among such gases as fluorocarbons, hydrocarbons, nitrogen and dinitrogen oxide or mixtures thereof.

The inhalation delivery device can be a nebulizer or a metered dose inhaler (MDI), or any other suitable inhalation delivery device known to one of ordinary skill in the art. The device can contain and be used to deliver a single dose of the formulations or the device can contain and be used to deliver multi-doses of the compositions of the present invention.

A nebulizer type inhalation delivery device can contain the compositions of the present invention as a solution, usually aqueous, or a suspension. In generating the nebulized spray of the compositions for inhalation, the nebulizer type delivery device may be driven ultrasonically, by compressed air, by other gases, electronically or mechanically. The ultrasonic nebulizer device usually works by imposing a rapidly oscillating waveform onto the liquid film of the formulation via an electrochemical vibrating surface. At a given amplitude the waveform becomes unstable, whereby it disintegrates the liquids film, and it produces small droplets of the formulation. The nebulizer device driven by air or other gases operates on the basis that a high pressure gas stream produces a local pressure drop that draws the liquid formulation into the stream of gases via capillary action. This fine liquid stream is then disintegrated by shear forces. The nebulizer may be portable and hand held in design, and may be equipped with a self contained electrical unit. The nebulizer device may comprise a nozzle that has two coincident outlet channels of defined aperture size through which the liquid formulation can be accelerated. This results in impaction of the two streams and atomization of the formulation. The nebulizer may use a mechanical actuator to force the liquid formulation through a multiorifice nozzle of defined aperture size(s) to produce an aerosol of the formulation for inhalation. In the design of single dose nebulizers, blister packs containing single doses of the formulation may be employed.

In the present invention the nebulizer may be employed to ensure the sizing of particles is optimal for positioning of the particle within, for example, the pulmonary membrane.

A metered dose inhalator (MDI) may be employed as the inhalation delivery device for the compositions of the present invention. This device is pressurized (pMDI) and its basic structure comprises a metering valve, an actuator and a container. A propellant is used to discharge the formulation from the device. The composition may consist of particles of a defined size suspended in the pressurized propellant(s) liquid, or the composition can be in a solution or suspension of pressurized liquid propellant(s). The propellants used are primarily atmospheric friendly hydrofluorocarbons (HFCs) such as 134a and 227. Traditional chlorofluorocarbons like CFC-11, 12 and 114 are used only when essential. The device of the inhalation system may deliver a single dose via, e.g., a blister pack, or it may be multi dose in design. The pressurized metered dose inhalator of the inhalation system can be breath actuated to deliver an accurate dose of the lipid-containing formulation. To insure accuracy of dosing, the delivery of the formulation may be programmed via a microprocessor to occur at a certain point in the inhalation cycle. The MDI may be portable and hand held.

In another embodiment, the delivery system may be a transdermal delivery system, such as, for example, a hydrogel, cream, lotion, ointment, or patch. A patch in particular may be used when a timed delivery of weeks or even months is desired.

In another embodiment, parenteral routes of administration may be used. Parenteral routes involve injections into various compartments of the body. Parenteral routes include intravenous (iv), i.e. administration directly into the vascular system through a vein; intra-arterial (ia), i.e. administration directly into the vascular system through an artery; intraperitoneal (ip), i.e. administration into the abdominal cavity; subcutaneous (sc), i.e. administration under the skin; intramuscular (im), i.e. administration into a muscle; and intradermal (id), i.e. administration between layers of skin. The parenteral route is sometimes preferred over oral ones when part of the formulation administered would partially or totally degrade in the gastrointestinal tract. Similarly, where there is need for rapid response in emergency cases, parenteral administration is usually preferred over oral.

Method of Treating Influenza

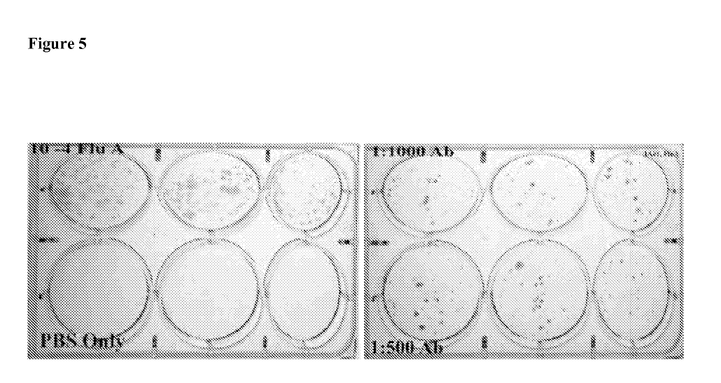

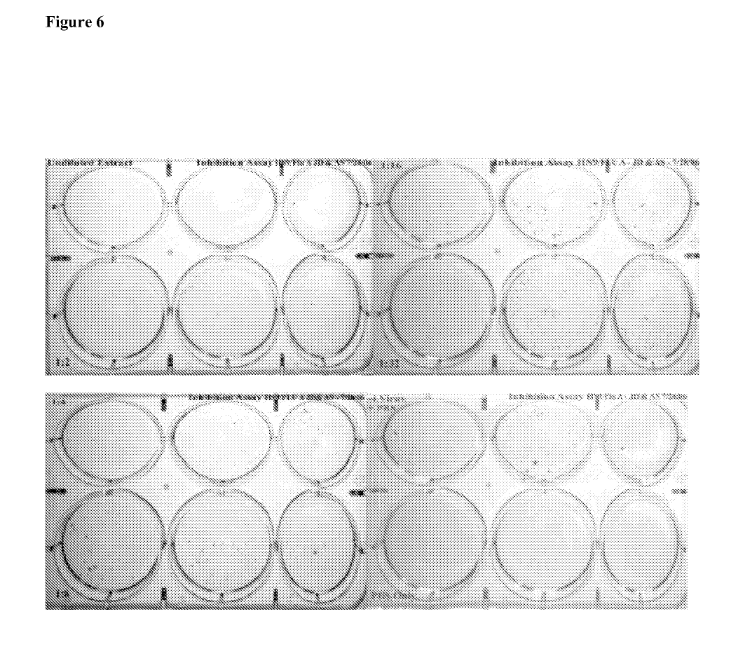

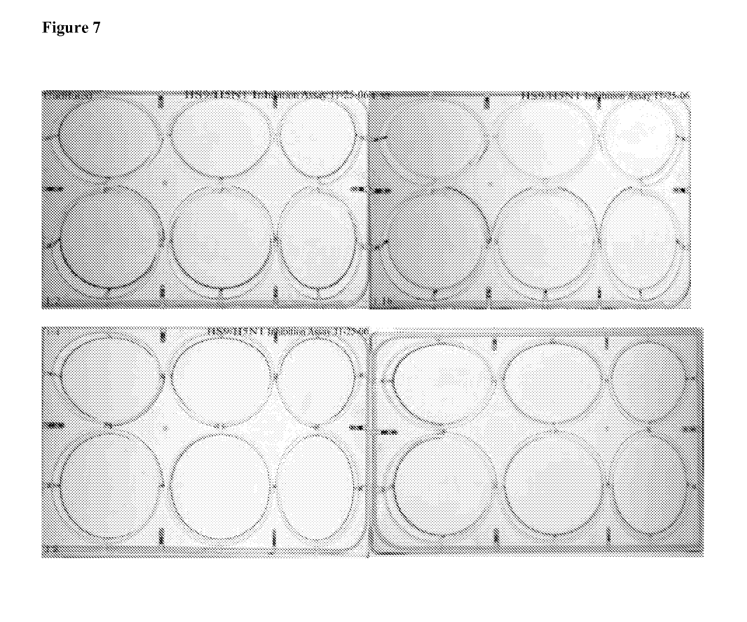

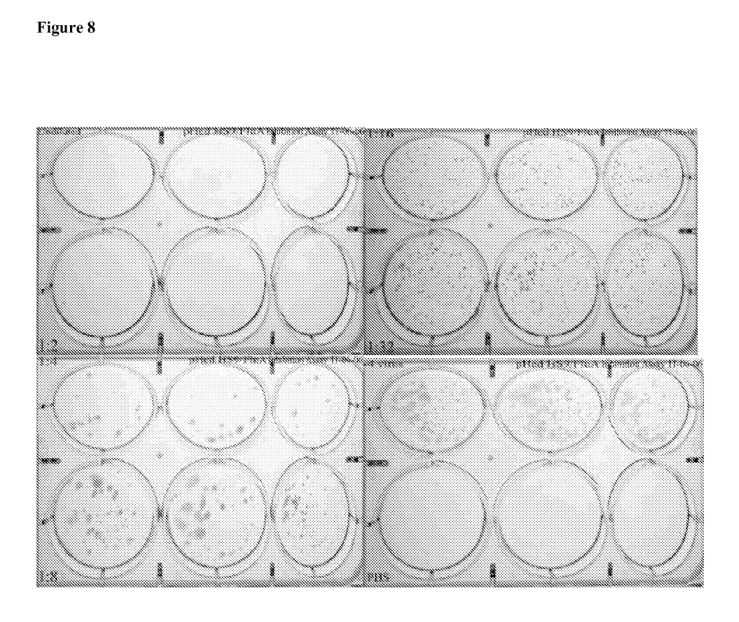

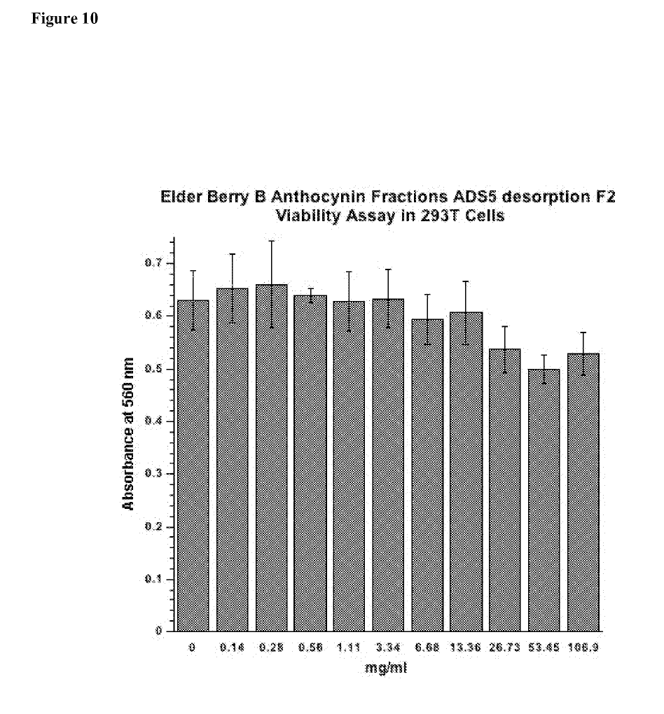

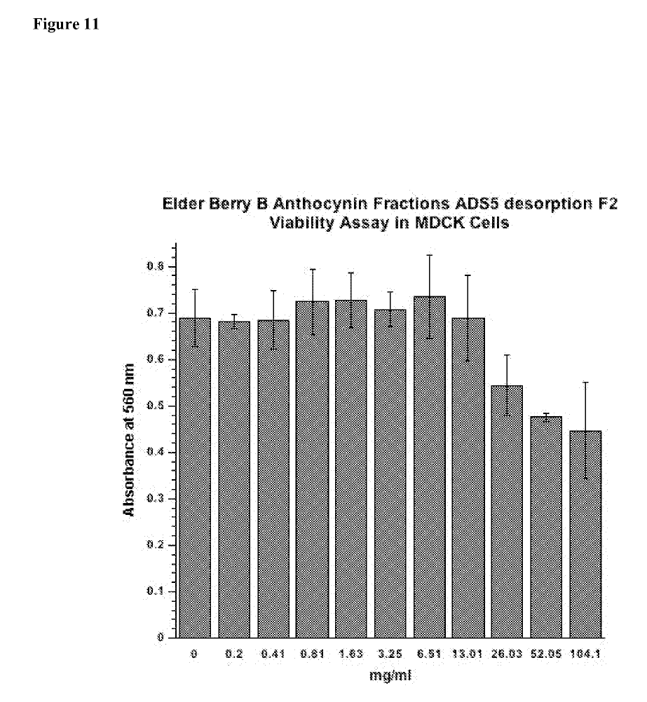

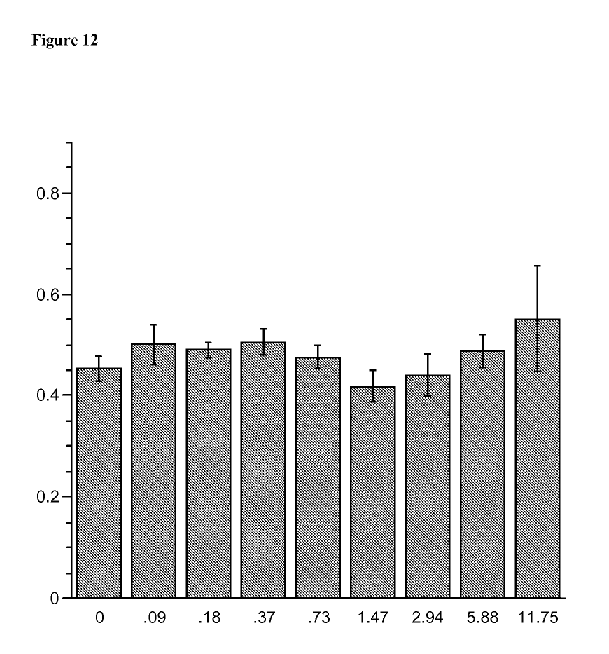

Inhibitory activity of elderberry fractions was quantified for influenza virus type A H1N1. Serial dilution of fractions were incubated with known quantities of virus and delivered to cell culture monolayers (see FIG. 5). Dose response curves were plotted and 50% inhibitory concentrations (IC₅₀) were determined for each fraction against human type A H1N1 virus. See FIGS. 6-11 and Table 16 below for IC₅₀ values. It has also been determined that the elderberry B anthocynin fractions ADS5 desorption F2 inhibits dengue virus as well as human influenza virus type A H1N1 (see FIG. 12). See Example 9 for the experimental protocol.

TABLE 16
Summary of inhibition analyses results
using human influenza type A H1N1 virus.

| Elderberry Fraction | IC₅₀ (μg/mL) |
|---|---|
| Elderberry B anthocyanin fraction ADS5 desorption F2 | 333 |
| Elderberry B anthocyanin fraction ADS5 desorption F3 | 521 |
| Elderberry B anthocyanin fraction ADS5 desorption F4 | 195 |
| Elder flower XAD 7HP desorption F2 | 1,592 |
| Elder flower XAD 7HP desorption F3 | 582 |

Method of Treating HIV

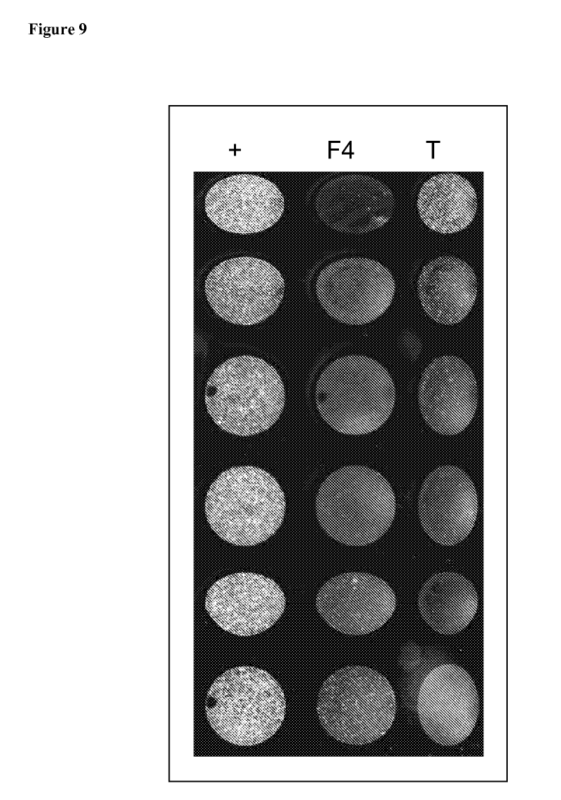

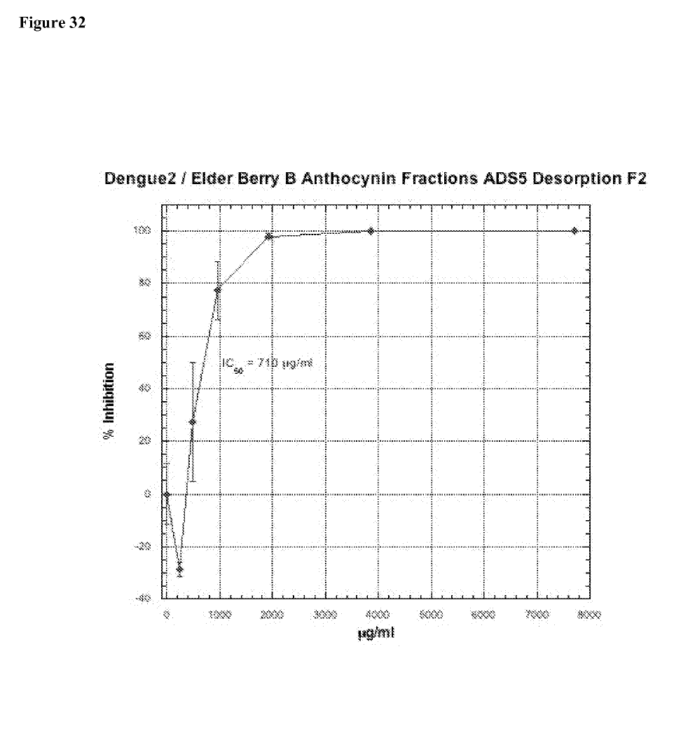

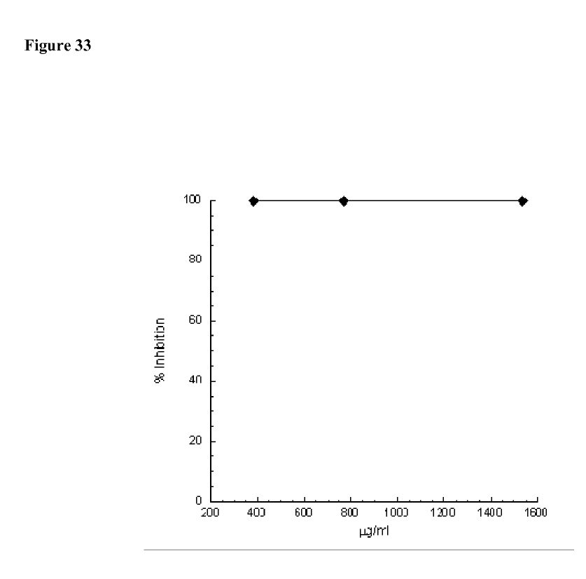

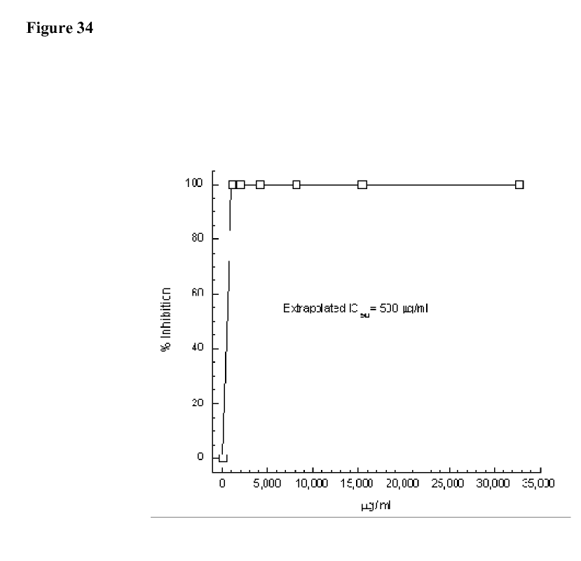

Inhibitory activity of elderberry fractions was quantified for HIV-1 virus. A known dilution of extraction was incubated with a known quantity of chimeric HIV-1 SG3 (genome) subtype C (envelope) virus. See FIG. 9. Dose response curves were plotted and extrapolated 50% inhibitory concentrations (IC₅₀) were determined. See FIGS. 32-34 and Table 17 below. See Example 10 for the experimental protocol.

TABLE 17
Summary of inhibition analyses results using HIV-1 virus.

| Trial | Cytoxicity observed at | IC₅₀ (μg/mL) |
|---|---|---|
| 1 | 8,182 μg/mL | 500 |
| 2 | 6,550 μg/mL | 153 |

### Exemplification

Materials

Botanicals: Wild crafted Sambucus nigra L. (elder) berries (Product #: 724, Lot #: L10379w, Hungary) and Sambucus nigra L. (elder) flowers (Product #: 725, Lot#: L01258W, Poland) were purchased from Blessed Herbs, Inc. Elder (Cincinnati).

Organic solvents: Acetone (67-64-1), >99.5%, ACS reagent (179124); Acetonitrile (75-05-8) for HPLC, gradient grade≧99.9% (GC) (000687); Hexane (110-54-3), 95+%, spectrophotometric grade (248878); Ethyl acetate (141-78-6), 99.5+%, ACS grade (319902); Ethanol, denatured with 4.8% isopropanol (02853); Ethanol (64-17-5), absolute, (02883); Methanol (67-56-1), 99.93%, ACS HPLC grade, (4391993); and Water (7732-18-5), HPLC grade, (95304). All were purchased from Sigma-Aldrich.

Acids and bases: Formic acid (64-18-6), 50% solution (09676); Acetic acid (64-19-7), 99.7+%, ACS reagent (320099); Hydrochloric acid (7647-01-0), volumetric standard 1.0N solution in water (318949); Folin-Ciocalteu phenol reagent (2N) (47641); Phenol (108-95-2) (P3653); Sulfuric acid (7664-93-9), ACS reagent, 95-97% (44719); and Sodium carbonate (S263-1, Lot #: 037406) were purchased from Fisher Co.
Chemical reference standards: Serum albumin (9048-46-8), Albumin Bovine Fraction V powder cell culture tested (A9418); Rutin (CAS# 153-18-4); and Cyanidin 3-glucoside chloride (CAS# 7084-24-4) were purchased from Chromadex. Dextran standards [5000 (00269), 50,000 (00891) and 410,000 (00895)] certified according to DIN were purchased from Fluka Co. The structures of the HPLC chemical reference standards are shown below. 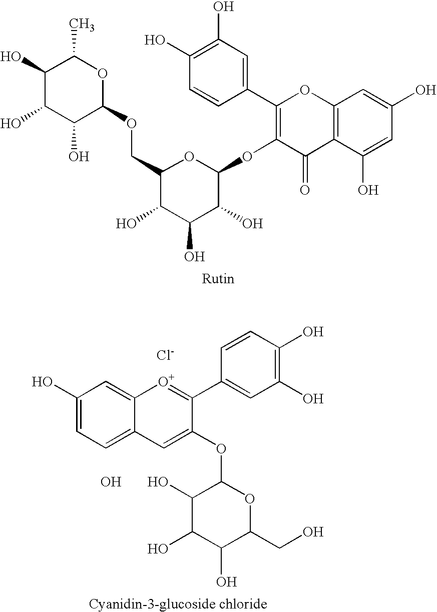
Polymer Affinity Adsorbents: Amberlite XAD 7HP (Rohm & Haas, France), macroreticular aliphatic acrylic cross-linked polymer used as white translucent beads with particle size of 560-710 nm and surface area is 380 m²/g. ADS-5 (Nankai University, China), ester group modified polystyrene with particle size of 300-1200 nm and surface area is 500-600 m²/g.
Methods
High Performance Liquid Chromatography (HPLC) Methods

Chromatographic system: Shimadzu high Performance Liquid Chromatographic LC-10AVP system equipped with LC10ADVP pump with SPD-M 10AVP photo diode array detector.

The ethanol extraction products of the present invention were measured on a reversed phase Jupiter C18 column (250×4.6 mm I. D., 5μ, 300 Å) (Phenomenex, Part #: 00G-4053-E0, serial No: 2217520-3, Batch No.: 5243-17). The injection volume was 10 μl and the flow rate of mobile phase was 1 ml/min. The column temperature was 25° C. The mobile phase consisted of A (5% formic acetic acid, v/v) and B (methanol). The gradient was programmed as follows: with the first 2 minutes, B maintains at 5%, 2-10 min, solvent B increased linearly from 5% to 24%, 10-15 min, B maintains at 24%, 15-30 min, B linearly from 24% to 35%, and 30-35 min, B maintains at 35%, 35-50 min, B linearly from 35% to 45%, held at this composition for five minutes, then 55-65 min, B linearly from 45% to 5%, 65-68 min, B maintains at 5%. Detection wavelengths were 350 nm for flavonoids and 520 nm for anthocyanidins.

Methanol stock solutions of the two reference standards were prepared by dissolving weighted quantities of standard compounds into ethanol at 5 mg/ml. The mixed reference standard solution was then diluted step by step to yield a series of solutions at final concentrations of 1.0, 0.5, 0.25, 0.1, and 0.05 mg/ml, respectively. All of the stock solutions and working solution were used within 7 days, stored in +4° C., and brought to room temperature before use. The solutions were used to identify and quantify the compounds in both elderberry and elder flower. Retention times of cyanidin-3-glucoside (CY3glu) at 520 nm and Rutin at 350 nm were about 13.27 and 20.20 min, respectively. A linear fit ranging from 0.01 to 20 μg was found. The regression equations and correlation coefficients were as follows: Anthocyanidin-3-glucoside: Area/100=20888*×C (μg)+502.21, R²=0.9994 (N=5); and Rutin: Area/100=11573×C (μg)+584.57, R²=0.9996 (N=5). HPLC results are shown in Table 18. The contents of the reference standards in each sample were calculated by interpolation from the corresponding calibration curves based on the peak area.

TABLE 18
HPLC analysis results of elder reference standards at concentration of 0.1 mg/ml in
methanol.

| | Retention | | | | Start | Stop | |
|---|---|---|---|---|---|---|---|
| | time | Area | Height | Width | time | time | Theoretical |
| ID | (min) | (mAu · min) | (mAu) | (min) | (min) | (min) | plate* |
| Cyanidin-3- | 13.312 | 1391742 | 104526 | 1.37 | 12.46 | 13.82 | 1510 |
| glucoside | | | | | | | |
| Rutin | 20.181 | 768924 | 21934 | 3.69 | 19.32 | 23.01 | 479 |

*Theoretical plates was calculated by: N = 16 × (t_{R}/w)² . t_{R} is retention time and w is width of the peak, http://www.mn-net.com/web%5CMN-WEB HPLCKatalog.nsf/WebE/GRUNDLAGEN

Gas Chromatography-Mass Spectroscopy (GC-MS) Methods

GC-MS analysis was performed using a Shimadzu GCMS-QP2010 system. The system includes high-performance gas chromatograph, direct coupled GC/MS interface, electro impact (E1) ion source with independent temperature control, and quadrupole mass filter. The system is controlled with GCMS solution Ver. 2 software for data acquisition and post run analysis. Separation was carried out on a Agilent J&W DB-5 fused silica capillary column (30 m×0.25 mm i.d., 0.25 μm film (5% phenyl, 95% dimethylsiloxane) thickness) (catalog: 1225032, serial No: U.S. Pat. No. 5,285,774H) using the following temperature program. The initial temperature was 60<c, held for 2 min, then it increased to 120° C. at rate of 4° C./min, held for 15 min, then it increased to 200<C at rate of 4° C./min, held for 15 min, then it increased to 240° C. at rate of 4° C./min, held another 15 min. The total run time was approximately 92 minutes. The sample injection temperature was 250° C. 1 μl of sample was injected by an auto injector at splitless mode in 1 minute. The carrier gas was helium and flowrate was controlled by pressure at 60 KPa. Under such pressure, the flow rate was 1.03 ml/min and linear velocity was 37.1 cm/min and total flow was 35 ml/min. MS ion source temperature was 230° C., and GC/MS interface temperature was 250° C. MS detector was scanned between m/z of 50 and 500 at scan speed of 1000 AMU/second with an ionizing voltage at 70 eV. Solvent cutoff temperature was 3.5 min.

Total phenolic acid concentration by Folin-Ciocalteu method (Markar, H. P. S., Bluemmel M. Borowy, N, K. and Becker, K., 1993, J. Sci. Food Agric. 61: 161-165)

Instruments: Shimadzu UV-V is spectrophotometer (UV 1700 with UV probe: S/N: A1102421982LP).

Reference Standards Make stock gallic acid/water solution at concentration of 1 mg/ml. Load suitable amounts of gallic acid solution into test tubes, make up the volume to 0.5 ml with distilled water, add 0.25 ml of the Folin Ciocalteu reagent, and then 1.25 ml of the 20 wt % sodium carbonate solution. Shake the tube well in an ultra-sonic bath for 40 min and record absorbance at 725 nm. The reference standard data are shown in Table 19.

TABLE 19
Calibration curve data for gallic acid reference standard use in
Folin-Ciocalteu method.

| | Gallic acid | | | | Sodium | Absorb- |
|---|---|---|---|---|---|---|
| | solution | Gallic | Distilled | Folin | carbonate | ance |
| | (0.1 mg/ml) | acid | water | reagent | solution | at 725 |
| Tube | (ml) | (μg) | (ml) | (ml) | (ml) | mm* |
| Blank | 0.00 | 0 | 0.50 | 0.25 | 1.25 | 0.000 |
| 1 | 0.02* | 2 | 0.48* | 0.25 | 1.25 | 0.111 |
| 2 | 0.04 | 4 | 0.46 | 0.25 | 1.25 | 0.226 |
| 3 | 0.06 | 6 | 0.44 | 0.25 | 1.25 | 0.324 |
| 4 | 0.08 | 8 | 0.42 | 0.25 | 1.25 | 0.464 |
| 5 | 0.1 | 10 | 0.40 | 0.25 | 1.25 | 0.608 |

*amount of gallic acid solution is depending on the absorption information.

Unknown sample: Take suitable aliquots of the tannin-containing extract in test tubes, make up the volume to 0.5 ml with distilled water, add 0.25 ml of the Folin-Ciocalteu reagent and then 1.25 ml of the sodium carbonate solution. Vortex the tubes and record absorbance at 725 nm after 40 min. Calculate the amount of total phenols as gallic acid equivalent from the above calibration curve.
Protein Content Determination by Bradford Reagent Method
Instrument: Shimadzu UV-V is spectrophotometer (UV 1700 with UV probe: S/N: A1102421982LP)

Standard calibration curve: Prepare protein standards of appropriate concentrations in the same buffer as the unknown samples. In the present invention, deionized water may be substituted for the buffer. Make the BSA standards ranging from 0.1-1.4 mg/ml by serially diluting the 2 mg/ml BSA protein standard solution. Then, mix 0.1 ml BSA standard with 3 ml Bradford reagent. Vortex the mixture and let the samples incubate at room temperature for 5-45 minutes. Record the absorbance at 595 nm. The absorbance of the samples must be recorded before the 60 minutes time limit and within 10 minute of each other. The results are shown in Table 20.

TABLE 20
Standard calibration data for Bradford protein assay.

| | BSA | | | | | |
|---|---|---|---|---|---|---|
| | standard | BSA | | | | Absorb |
| | concen- | solution | Distilled | Bradford | BSA | ance |
| Tube | tration | (2 mg/ml) | water | reagent | amount | at 595 |
| No. | (mg/ml) | (μl) | (μl) | (ml) | (μg) | nm |
| 0 | 0 | 0 | 100 | 3 | 0 | 0.415 |
| 1 | 0.1 | 5 | 95 | 3 | 10 | 0.497 |
| 2 | 0.3 | 15 | 85 | 3 | 30 | 0.672 |
| 3 | 0.5 | 25 | 75 | 3 | 50 | 0.818 |
| 4 | 1.0 | 50 | 50 | 3 | 100 | 1.169 |

Analysis of unknown samples: Take suitable aliquots of the protein-containing test samples in test tubes; make up the volume to 0.1 ml with distilled water. Then add 3 ml Bradford reagent. Shake the tube and record absorbance at 595 nm within 5-45 minutes. Calculate the amounts of protein as BSA standard equivalent from above calibration curve.
Polysaccharide analysis using colormetric method (Dubois, M., Gilles, K. A., Hamilton, J. K., Rebers, P. A. and Smith, F., 1956, Analytical Chemistry 28(3): 350-356).

Spectrophotometer system: Shimadzu UV-1700 ultraviolet visible spectrophotometer (190-1100 nm, 1 mm resolution) has been used in this study.

Colorimetric method has been used for polysaccharide analysis. Make 0.1 mg/ml stock dextran (Mw=5000, 50,000 and 410,000) solutions. Take 0.08, 0.16, 0.24, 0.32, 0.40 ml of stock solution and make up volume to 0.4 ml with distilled water. Then add in 0.2 ml 5% phenol solution and 1 ml concentrated sulfuric acid. The mixtures were allowed to stand for 10 minutes prior to performing UV scanning. The maximum absorbance was found at 488 nm. Then set the wavelength at 488 nm and measure absorbance for each sample. The results are shown in Table 21. The standard calibration curves were obtained for each of the dextran solutions as follows: Dextan 5000, Absorbance=0.01919+0.027782 C (μg), R²=0.97 (N=5); Dextan 50,000, Absorbance=0.0075714+0.032196 C (μg), R²=0.96 (N=5); and Dextan 410,000, Absorbance=0.03481+0.036293C (μg), R²=0.98 (N=5).

TABLE 21
Colorimetric analysis of dextran reference standards.

| | Dextran | Distill | 5% | Sulfuric | | | |
|---|---|---|---|---|---|---|---|
| | solution | water | phenol | acid | Abs | Abs | |
| Tube | (ml) | (ml) | (ml) | (ml) | (Mw = 5 K) | (Mw = 50 K) | Abs (Mw =410 K) |
| blank | 0 | 0.40 | 0.2 | 1 | 0 | 0 | 0 |
| 1 | 0.08 | 0.32 | 0.2 | 1 | 0.238 | 0.301 | 0.335 |
| 2 | 0.16 | 0.24 | 0.2 | 1 | 0.462 | 0.504 | 0.678 |
| 3 | 0.24 | 0.16 | 0.2 | 1 | 0.744 | 0.752 | 0.854 |
| 4 | 0.32 | 0.08 | 0.2 | 1 | 0.907 | 1.045 | 1.247 |
| 5 | 0.40 | 0.00 | 0.2 | 1 | 1.098 | 1.307 | 1.450 |

Direct Analysis in Real Time (DART) Mass Spectrometry for Polysaccharide Analysis.

All DART chromatograms, and in particular those for fractions F1-F6 from XAD 7HP packing material and fractions F1-F4 from ADS5 packing material, were run using the instruments and methods described below.

Instruments: JOEL AccuTOF DART LC time of flight mass spectrometer (Joel USA, Inc., Peabody, Massachusetts, USA). This Time of Flight (TOF) mass spectrometer technology does not require any sample preparation and yields masses with accuracies to 0.00001 mass units.

Methods: The instrument settings utilized to capture and analyze polysaccharide fractions are as follows: For cationic mode, the DART needle voltage is 3000 V, heating element at 250<c, Electrode 1 at 100 V, Electrode 2 at 250 V, and helium gas flow of 7.45 liters/minute (L/min). For the mass spectrometer, orifice 1 is 10 V, ring lens is 5 V, and orifice 2 is 3 V. The peaks voltage is set to 600 V in order to give resolving power starting an approximately 60 m/z, yet allowing sufficient resolution at greater mass ranges. The micro-channel plate detector (MCP) voltage is set at 2450 V. Calibrations are performed each morning prior to sample introduction using a 0.5 M caffeine solution standard (Sigma-Aldrich Co., St. Louis, USA). Calibration tolerances are held to <5 mmu.

The samples are introduced into the DART helium plasma with sterile forceps ensuring that a maximum surface area of the sample is exposed to the helium plasma beam. To introduce the sample into the beam, a sweeping motion is employed. This motion allows the sample to be exposed repeatedly on the forward and back stroke for approximately 0.5 sec/swipe and prevented pyrolysis of the sample. This motion is repeated until an appreciable Total Ion Current (TIC) signal is observed at the detector, then the sample is removed, allowing for baseline/background normalization.

For anionic mode, the DART and AccuTOF MS are switched to negative ion mode. The needle voltage is 3000 V, heating element 250<C, Electrode 1 at 100 V, Electrode 2 at 250 V, and helium gas flow at 7.45 L/min. For the mass spectrometer, orifice 1 is −20 V, ring lens is −13 V, and orifice 2 is −5 V. The peak voltage is 200 V. The MCP voltage is set at 2450V. Samples are introduced in the exact same manner as cationic mode. All data analysis is conducted using MassCenterMain Suite software provided with the instrument.

### EXAMPLE 1

Example of Step 1A: Single Step SFE Maximal Extraction and Purification of Elderberry.

All SFE extractions were performed on SFT 250 (Supercritical Fluid Technologies, Inc., Newark, Del., USA) designed for pressures and temperatures up to 690 bar and 200° C., respectively. This apparatus allows simple and efficient extractions at supercritical conditions with flexibility to operate in either dynamic or static modes. This apparatus consists of mainly three modules; an oven, a pump and control, and collection module. The oven has one preheat column and one 100 ml extraction vessel. The pump module is equipped with a compressed air-driven pump with constant flow capacity of 300 ml/min. The collection module is a glass vial of 40 ml, sealed with caps and septa for the recovery of extracted products. The equipment is provided with micrometer valves and a flow meter. The extraction vessel pressure and temperature are monitored and controlled within [symbol] 3 bar and [symbol] 1° C.

In typical experimental examples, 5 grams of either ground Sambucus Nigra L berry (elderberry) or flower (elder flower) powder with size above 105 μm sieved measured using a screen (140 mesh) was loaded into a 100 ml extraction vessels for each experiment. Glass wool was placed at the two ends of the column to avoid any possible carry over of solid material. The oven was preheated to the desired temperature before the packed vessel was loaded. After the vessel was connected into the oven, the extraction system was tested for leakage by pressurizing the system with CO₂ (˜850 psig), and purged. The system was closed and pressurized to the desired extraction pressure using the air-driven liquid pump. The system was then left for equilibrium for ˜3 min. A sampling vial (40 ml) was weighed and connected to the sampling port. The extraction was started by flowing CO₂ at a rate of ˜5 SLPM (10 g/min), which is controlled by a meter valve. The yield was defined to be the weight ratio of total exacts to the feed of raw material. The yield was defined as the weight percentage of the oil extracted with respect to the initial charge of the raw material in the extractor. A full factorial extraction design was adopted varying the temperature from 40-80° C. and from 100-500 bar. The extracts obtained at each condition were dissolved in dichloromethane at concentration of 400 ppm for Gas Chromatography-Mass Spectroscopy (GC-MS) analysis.

### EXAMPLE 2

Example of Step 2: Hydroalcoholic Leaching Extraction.

A typical example of a 2 stage solvent extraction of the phenolic acid chemical constituents of elder species is as follows: The feedstock was 17.6 gm of ground elderberry SFE residue from Step 1 SCCO₂ extraction (60<c, 300 bar, 90 min) of the essential oil. The solvent was 300 ml of 25% aqueous ethanol. In this method, the feedstock material and 80% aqueous ethanol were separately loaded into 500 ml extraction vessel and mixed in a heated water bath at 60<C for 4 hours. The extraction solution was filtered using Fisherbrand P4 filter paper having a particle retention size of 4-8 μm, centrifuged at 2000 rpm for 20 minutes, and the particulate residue used for further extraction. The filtrates (supernatants) were collected and combined for yield calculation, HPLC analysis, and production of F1-F4 and F1-F6 fractions (see Example 3 below). The residue of Stage 1 was extracted for 2 hours (Stage 2) using the aforementioned methods.

### EXAMPLE 3

Example of Step 3 Affinity Adsorbent Extraction of Phenolic Acid Fraction (Preparation of F1-F4 and F1-F6 Fractions).

In typical experiments, the working solution was the transparent hydroalcoholic solution of elder species aqueous ethanol leaching extract in Step 2. The affinity adsorbent polymer resin was XAD7HP or ADS5. 15 gm of ADS5 affinity adsorbent or 20 gm of XAD7HP affinity adsorbent was pre-washed with 95% ethanol (4-5 BV) and distilled water (4-5 BV) before and after packing into a column with an ID of 25 mm and length of 500 mm. The loading solutions were the crude 80% ethanol leaching phenolic acid solutions wherein the chemical constituents were concentrated by rotary vacuum distillation and recycling of the ethanol. The final loading solution concentration was 29.03 mg/ml for XAD7HP loading and 34.90 mg/ml for ADS5 loading. 50 ml loading solution was loaded on the XAD7HP column and 60 ml of loading solution was loaded on the ADS5 column at a flow rate of 0.3 BV/hr. The loading time was about 50-60 minutes. The loaded column washed with 2 BV of distilled water at a flow rate of 0.2 BV/hr with a washing time of 13 minutes. 40 ml of 40% and 80% aqueous ethanol was used to sequentially elute the loaded column at a flow rate of 2 ml/min for XAD7HP and 1.5 ml/min for ADS5. During the elution, 6 eluant fractions (F1-F6: F1˜20 mL, F2˜20 mL, F3˜18 mL, F4˜10 mL, F5˜17 mL, and F6˜27 mL) were collected from the XAD7HP column and 4 eluant fractions (F1-F4: F1˜20 mL, F2˜20 mL, F3˜17 mL, and F4˜17 mL) from the ADS5 column, respectively. For the XAD7HP column, F1-F3 were eluted using 40% ethanol and F4-F6 were collected using 80% ethanol. For the ADS5 column, F1-F2 were eluted using 40% ethanol and F3-F4 were eluted using 80% ethanol. Then 4-5 BV of 95% ethanol was used to clean out the remaining chemicals on the column at a flow rate of 3.6 BV/hr followed by washing with 4-5 BV distilled water at 3.8 BV/hr. The total processing time was less than 2 hours. The flow rate during whole process was controlled using a FPU 252 Omegaflex® variable speed (3-50 ml/min) peristaltic pump. Each elution fraction was collected and analyzed by DART mass balance and HPLC.

### EXAMPLE 4

Example of Step 5 Polysaccharide Fraction Extraction

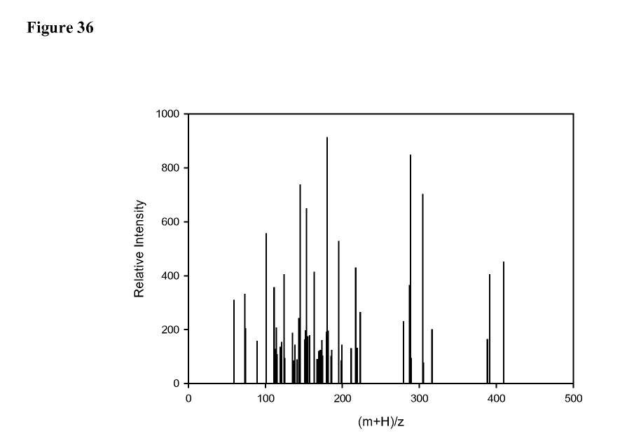

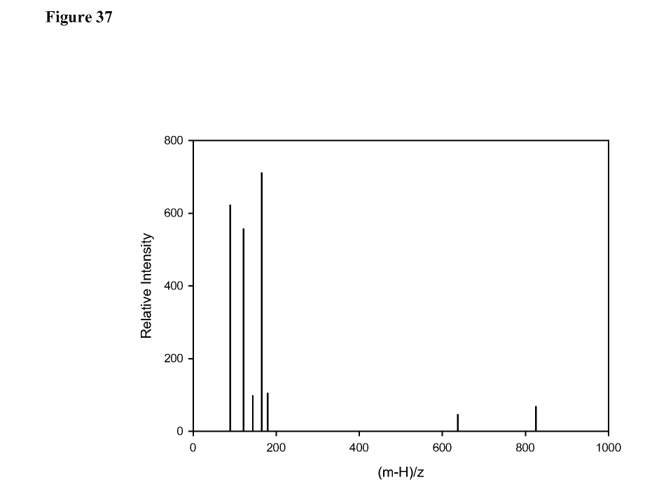

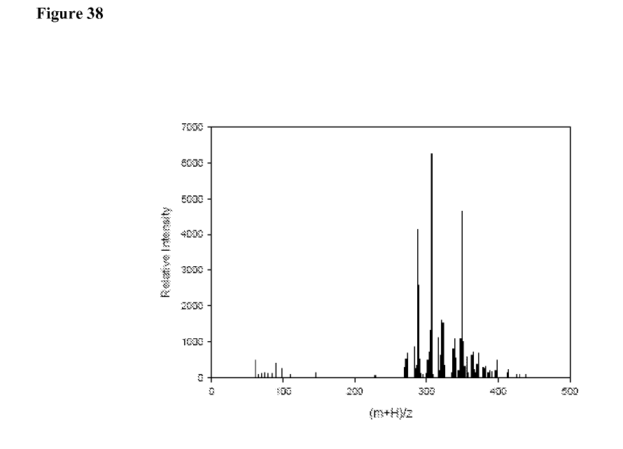

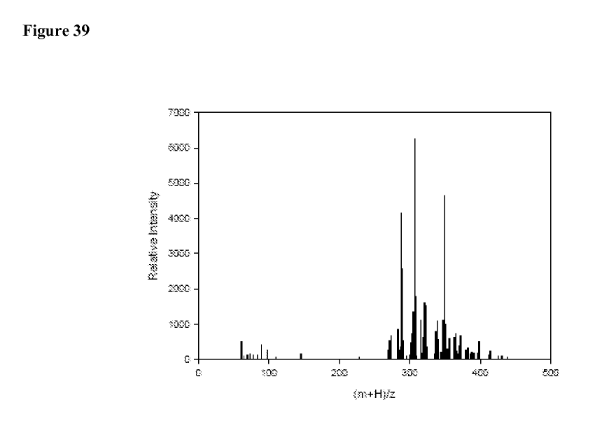

A typical experimental example of solvent extraction and precipitation of the water soluble, ethanol insoluble purified lectin-polysaccharide fraction chemical constituents of elder species is as follows: 15 gm of the solid residue from the 2 stage hydroalcoholic leaching extraction (Step 2) was extracted using 300 ml of distilled water for two hours at 80<C in two stages. The two extraction solutions were combined and the slurry was filtered using Fisherbrand P4 filter paper (pore size 4-8 μm) and centrifuged at 2,000 rpm for 20 minutes. The concentration of compounds in solution was 3.8 mg/ml. 300 ml of this solution and then, 456 ml or 1200 ml of anhydrous ethanol was added to make up a final ethanol concentration of 60% or 80%. The solutions were allowed to sit for 1 hour while precipitation occurred. The extraction solution was centrifuged at 3,000 rpm for 20 minutes and the supernatant decanted and discarded. The precipitate was collected and dried in an oven at 50° C. for 12 hours. The dried polysaccharide fraction was weighed and dissolved in water for analysis of polysaccharide purity with the colormetric method using dextran as reference standards and for analysis of lectin protein purity using the Bradford protein assay method. AccuTOF-DART mass spectrometry was used to further profile the molecular weights of the compounds comprising the purified polysaccharide fraction. The results for elderberry are shown in FIGS. 36 and 37 and Table 22. The results for elder flower are shown in FIGS. 38 and 39 and Table 22.

TABLE 20
DART analysis polysaccharide from elderberry and elder flower.

| Elderberry | | | | Elder flower | | | |
|---|---|---|---|---|---|---|---|
| positive ion | | negative ion | | positive ion | | negative ion | |
| | Relative | | Relative | | Relative | | Relative |
| (m + H)/z | Intensity | (m − H)/z | Intensity | (m + H)/z | Intensity | (m − H)/z | Intensity |
| 59.1 | 309.9 | 89.0 | 622.5 | 61.0 | 490.0 | 89.0 | 368.5 |
| 73.1 | 332.1 | 121.0 | 556.6 | 65.1 | 96.1 | 94.0 | 142.7 |
| 74.1 | 204.9 | 143.1 | 98.4 | 70.1 | 116.6 | 111.0 | 52.0 |
| 89.1 | 157.2 | 165.0 | 711.5 | 74.1 | 148.3 | 112.0 | 104.2 |
| 101.1 | 556.5 | 179.1 | 105.2 | 78.1 | 116.5 | 113.0 | 410.9 |
| 111.1 | 356.6 | 637.1 | 46.5 | 84.1 | 107.2 | 133.0 | 122.2 |
| 113.1 | 127.2 | 825.2 | 68.5 | 90.1 | 401.5 | 171.0 | 128.2 |
| 114.1 | 207.3 | | | 98.1 | 262.3 | 191.1 | 112.2 |
| 115.1 | 107.7 | | | 110.1 | 70.1 | | |
| 119.1 | 136.2 | | | 146.1 | 142.9 | | |
| 121.1 | 153.4 | | | 228.2 | 68.9 | | |
| 124.1 | 404.1 | | | 269.2 | 278.1 | | |
| 125.1 | 93.7 | | | 271.3 | 517.4 | | |
| 135.1 | 187.0 | | | 272.3 | 121.2 | | |
| 136.1 | 84.4 | | | 273.3 | 676.9 | | |
| 138.1 | 143.6 | | | 283.2 | 850.1 | | |
| 141.1 | 89.1 | | | 284.2 | 164.7 | | |
| 143.1 | 241.9 | | | 285.2 | 269.3 | | |
| 144.1 | 67.8 | | | 286.2 | 167.0 | | |
| 145.1 | 737.2 | | | 287.3 | 356.4 | | |
| 151.1 | 162.5 | | | 288.3 | 4144.0 | | |
| 152.1 | 196.1 | | | 289.3 | 2578.7 | | |
| 153.1 | 649.2 | | | 290.3 | 521.0 | | |
| 155.1 | 174.0 | | | 291.3 | 112.9 | | |
| 157.1 | 178.8 | | | 295.2 | 90.8 | | |
| 163.1 | 413.8 | | | 300.3 | 112.7 | | |
| 167.1 | 90.3 | | | 301.2 | 472.6 | | |
| 169.1 | 120.4 | | | 302.2 | 200.1 | | |
| 171.1 | 123.5 | | | 303.2 | 719.0 | | |
| 173.1 | 159.9 | | | 305.3 | 1332.0 | | |
| 174.1 | 102.4 | | | 306.3 | 361.8 | | |
| 179.1 | 191.2 | | | 307.3 | 6262.5 | | |
| 180.2 | 912.9 | | | 308.3 | 1781.9 | | |
| 181.1 | 195.4 | | | 309.3 | 95.0 | | |
| 185.1 | 102.0 | | | 316.3 | 1114.4 | | |
| 186.1 | 123.7 | | | 317.3 | 189.6 | | |
| 195.1 | 528.5 | | | 319.2 | 627.1 | | |
| 198.1 | 85.0 | | | 320.3 | 247.6 | | |
| 199.2 | 143.6 | | | 321.2 | 1612.0 | | |
| 211.1 | 130.5 | | | 322.3 | 521.6 | | |
| 217.2 | 428.7 | | | 323.3 | 1510.2 | | |
| 219.2 | 131.2 | | | 324.3 | 358.6 | | |
| 223.1 | 264.7 | | | 335.2 | 140.7 | | |
| 279.2 | 229.8 | | | 337.3 | 805.3 | | |
| 287.2 | 365.1 | | | 338.3 | 429.6 | | |
| 288.3 | 848.4 | | | 339.3 | 1079.5 | | |
| 289.3 | 93.5 | | | 340.3 | 546.7 | | |
| 304.2 | 703.1 | | | 344.3 | 192.4 | | |
| 305.2 | 77.7 | | | 347.3 | 1100.0 | | |
| 316.3 | 200.2 | | | 348.3 | 235.6 | | |
| 371.1 | 534.9 | | | 349.3 | 4638.4 | | |
| 372.1 | 130.1 | | | 350.3 | 1002.4 | | |
| 373.1 | 107.3 | | | 351.3 | 113.1 | | |
| 388.1 | 164.0 | | | 353.3 | 306.8 | | |
| 391.3 | 405.3 | | | 354.3 | 238.3 | | |
| 409.4 | 451.1 | | | 355.3 | 417.2 | | |
| | | | | 356.3 | 584.2 | | |
| | | | | 357.3 | 134.8 | | |
| | | | | 363.3 | 628.0 | | |
| | | | | 364.3 | 127.8 | | |
| | | | | 365.3 | 725.6 | | |
| | | | | 366.3 | 243.5 | | |
| | | | | 367.3 | 108.1 | | |
| | | | | 368.3 | 141.9 | | |
| | | | | 370.3 | 378.9 | | |
| | | | | 372.3 | 686.7 | | |
| | | | | 379.3 | 278.8 | | |
| | | | | 380.3 | 70.5 | | |
| | | | | 381.3 | 252.7 | | |
| | | | | 382.3 | 330.0 | | |
| | | | | 386.3 | 141.3 | | |
| | | | | 388.3 | 198.4 | | |
| | | | | 391.3 | 167.3 | | |
| | | | | 396.3 | 188.3 | | |
| | | | | 397.3 | 138.7 | | |
| | | | | 398.3 | 501.2 | | |
| | | | | 412.3 | 133.1 | | |
| | | | | 414.3 | 235.2 | | |
| | | | | 425.4 | 85.7 | | |
| | | | | 430.3 | 89.8 | | |
| | | | | 438.3 | 70.7 | | |

### EXAMPLE 5

The following Ingredients are Mixed for the Formulation:

| | |
|---|---|
| Extract of S. nigra L. berries | 150.0 mg |
| Essential Oil Fraction (10 mg, 6.6% dry weight) | |
| Polyphenolic Fraction (120 mg, 80% dry weight) | |
| Polysaccharides (40 mg, 26.6% dry weight) | |
| Stevioside (Extract of Stevia) | 12.5 mg |
| Carboxymethylcellulose | 35.5 mg |
| Lactose | 77.0 mg |
| Total | 275.0 mg |

The novel extract of elder species comprises an essential oil fraction, phenolic acid-essential oil fraction, and polysaccharide fraction by % mass weight greater than that found in the natural rhizome material or convention extraction products. The formulations can be made into any oral dosage form and administered daily or to 15 times per day as needed for the physiological and psychological effects desired (reduction of agitation and restlessness) and medical effects (viral diseases such as the common cold, influenza, herpes simplex, herpes zoster, and HIV, diabetes mellitus, cardiovascular and cerebrovascular disease prevention and treatment, anti-atherosclerosis, anti-oxidant and free radical scavenging, anti-inflammatory, anti-arthritis, anti-rheumatic, and gastro-intestinal disorders).

### EXAMPLE 6

The Following Ingredients were Mixed for the Following Formulation:

| | |
|---|---|
| Extract of S. nigra L. berries | 150.0 mg |
| Essential Oil Fraction (6 mg, 4% dry weight) | |
| Polyphenolic Fraction (30 mg, 20% dry weight) | |
| Polysaccharides (114.0 mg, 76% dry weight) | |
| Vitamin C | 15.0 mg |
| Sucralose | 35.0 mg |
| Mung Bean Powder 10:1 | 50.0 mg |
| Mocha Flavor | 40.0 mg |
| Chocolate Flavor | 10.0 mg |
| Total | 300.0 mg |

The novel extract composition of elder chuangxiong comprises an essential oil, phenolic acid-essential oil, and polysaccharide chemical constituent fractions by % mass weight greater than that found in the natural plant material or conventional extraction products. The formulation can be made into any oral dosage form and administered safely up to 15 times per day as needed for the physiological, psychological and medical effects desired (see Example 5, above).

### EXAMPLE 7

MTT Assay for Determination of Cell Number to be Used

Purpose: This is a control experiment to determine amount of cells to use in future MTT/cytotoxicity assays. It should only need to be done once per cell line used.

JD Evaluation of Bioactives for Antiviral Activity

Day One

From one confluent T-75 flask of cells (this protocol was written using MDCKs):
1. Aspirate off media and add 2 mL of trypsin to flasks. Inc. 5 min. at 37° C.
2. Hit the sides of the flasks with force and remove trypsin to a 50 cc conical tube. Add 0.5 mL growth media (DMEM+P/S+Glutamax+FBS) to this tube also.
3. Add another 2 mL trypsin to the flask. Inc. 3-5 min. at 37° C.
4. Hit the sides of the flasks with force and remove trypsin to the 50 cc tube from step 2. Add 10 mL growth medium to the flask, rinsing flask bottom 2 times. Put this 10 mL media into the same 50 cc tube. Check flask using microscope to see if cells are removed.
5. Spin down at 4° C., 1000 rpm for 5 min. Aspirate off supernatant.
6. Dislodge the pellet and resuspend the pellet in 5 mL growth medium.
7. Spin down at 4° C., 1000 rpm for 5 min. Aspirate off supernatant.
8. Dislodge the pellet and resuspend the cells in 1 mL growth medium.
9. Dilute cells 1:2 by adding 500 μl cells to 500 μl growth medium in a microfuge tube. If you started with a plate that was extremely high in cell density, you may want to dilute cells 1:4 in growth medium.
10. Check 10 μl of diluted cells on hemacytometer. Record the cell count for 3 large grids and take the average of these three numbers. This gives you the cell count: average×10⁴ cells/mL. You want to start with about 5×10⁶ cells/mL. If you have too many cells, re-count cells after another dilution.

11. Use a total of 11 microfuge tubes to set up 2-fold dilutions. Here is an example:

| Tube # | Cells/mL | Add Medium | Add Cells |
|---|---|---|---|
| 1 | 1.34 × 10⁶ | — | — |
| 2 | 6.7 × 10⁵ | 400 μl | 400 μl from tube 1 |
| 3 | 3.35 × 10⁵ | 400 μl | 400 μl from tube 2 |
| 4 | 1.68 × 10⁵ | 400 μl | 400 μl from tube 3 |
| 5 | 8.4 × 10⁴ | 400 μl | 400 μl from tube 4 |
| 6 | 4.2 × 10⁴ | 400 μl | 400 μl from tube 5 |
| 7 | 2.1 × 10⁴ | 400 μl | 400 μl from tube 6 |
| 8 | 1.05 × 10⁴ | 400 μl | 400 μl from tube 7 |
| 9 | 5.25 × 10³ | 400 μl | 400 μl from tube 8 |
| 10 | 2.63 × 10³ | 400 μl | 400 μl from tube 9 |
| 11 | Media only control | 400 μl | — |

12. This assay is done in triplicate, so add 100 μl from each tube into wells A-C in a 96-well plate, with each column number in the plate corresponding to the tube whose sample it now contains.
13. Incubate plate at 37° C. overnight w/CO₂, or as long as it takes for cells to recover and reattach (usually 12-18 hours).
   Day Two
1. Around 9:00 a.m., check the cells in the plate under the microscope to be sure that they are adherent, that they are confluent at least in column 1, and that you see less cells per well as you move across the plate. The media in the first 2-3 columns should be orange; others should be pink.
2. Add 10 μl MTT reagent (which is stored at 4° C.) per well, changing tips between each well and being careful not to contaminate the stock of MTT reagent. Incubate plate at 37° C. for 2 hours.
3. Check plate under microscope for the appearance of purple punctate, intracellular precipitate. If you don't see this, continue incubation for up to 24 hours.
4. Once you see the precipitate, add 100 μl detergent reagent (stored at room temp) per well. DO NOT SHAKE THE PLATE FROM HERE ON OUT. Cover plate with aluminum foil and leave plate at room temp overnight.
   Day Three
1. Using a Tecan plate reader, measure the absorbance of the wells at 560 nm with a reference wavelength of 620 nm. You will do this if you use any of the programs called “MTT” in XFluor4. You will need to be sure that filter slide C is in the Tecan.
2. Determine the average values from triplicate readings and subtract the average value from the average for the medium-only blank (column 11). Plot absorbance on the y-axis and cell number per mL on the x-axis.
   Select a cell number for use in future assays that yields an absorbance of 0.75 to 1.25. The cell number selected should fall in the linear portion of the curve.

### EXAMPLE 8

MTT Assay

Purpose: To determine if extract(s) have cytotoxic effects on cells.

JD Evaluation of Bioactives for Antiviral Activity

Day One

1. Using the ultra-sensitive balance by the window in WH265, measure out 0.01 g of extract and dissolve in 100 μl sterile PBS. You will drive yourself crazy trying to make this exact, so get it as close as you can and record mass in your notebook, along with extract tube label details. This is your “undiluted extract” and is in concentration of about 0.1 g/mL. If the extract is not completely soluble, spin down precipitate in microcentrifuge at 13k rpm for 30 sec., remove supernatant to a sterile microfuge tube to work with today, and store pellet at −20° C. for possible future use.

From one confluent T-75 flask of cells (this protocol was written using MDCKs):
1. Aspirate off media and add 2 mL of trypsin to flasks. Inc. 5 min. at 37° C.
2. Hit the sides of the flasks with force and remove trypsin to a 50 cc conical tube. Add 0.5 mL growth media (DMEM+P/S+Glutamax+FBS) to this tube also.
3. Add another 2 mL trypsin to the flask. Inc. 3-5 min. at 37° C.
4. Hit the sides of the flasks with force and remove trypsin to the 50 cc tube from step 2. Add 10 mL growth medium to the flask, rinsing flask bottom 2 times. Put this 10 mL media into the same 50 cc tube. Check flask using microscope to see if cells are removed.
5. Spin down at 4° C., 1000 rpm for 5 min. Aspirate off supernatant.
6. Dislodge the pellet and resuspend the pellet in 5 mL growth medium.
7. Spin down at 4° C., 1000 rpm for 5 min. Aspirate off supernatant.
8. Dislodge the pellet and resuspend the cells in 1 mL growth medium.
9. Dilute cells 1:2 by adding 500 μl cells to 500 μl growth medium in a microfuge tube. If you started with a plate that was extremely high in cell density, you may want to dilute cells 1:4 in growth medium.
10. Check 10 μl of diluted cells on hemacytometer. Record the cell count for 3 large grids and take the average of these three numbers. This gives you the cell count: average×10⁴ cells/mL. To start, there should be about 1-1.6×10⁵ MDCK cells/mL or 1.3-2.1×10⁵ 293T cells/mL; this can be achieved by the following:
   For MDCKs:
   a. Dilute 1:4
   b. Count cells. You'll usually get about 360 cells per big grid.
   c. Dilute your 1:4 1:3. Then dilute that 1:10 (400 μl cells in 3.6 mL media).
   d. Count cells. You want 10-16 cells per big grid.
   For 293Ts:
   a. Dilute 1:8.
   b. Count cells. You'll usually get about 300 cells per big grid.
   c. Dilute your 1:8 1:2. Then dilute that 1:10 (400 μl cells in 3.6 mL media).
   d. Count cells. You want 13-21 cells per big grid.

11. Use a total of 9 microfuge tubes to set up 2-fold dilutions of extract as follows:

| Tube # | Extract Dilution | Add PBS | Add Extract |
|---|---|---|---|
| 1 | Undiluted | — | — |
| 2 | 1:2 | 50 μl | 50 μl from tube 1 |
| 3 | 1:4 | 50 μl | 50 μl from tube 2 |
| 4 | 1:8 | 50 μl | 50 μl from tube 3 |
| 5 | 1:16 | 50 μl | 50 μl from tube 4 |
| 6 | 1:32 | 50 μl | 50 μl from tube 5 |
| 7 | 1:64 | 50 μl | 50 μl from tube 6 |
| 8 | 1:128 | 50 μl | 50 μl from tube 7 |
| 9 | 1:256 | 50 μl | 50 μl from tube 8 |
| 10 | 1:512 | 50 μl | 50 μl from tube 9 |

In 96-well plate, column
11=PBS/solvent only control (has cells but no extract)=
12=Medium only control (blank—no cells, no extract)
12. This assay is done in triplicate, so add 100 μl of freshly-vortexed, properly diluted cells into rows A-C of columns 1-11 in a sterile 96-well plate, vortexing cells in tube after filling 3 columns.
13. Add 100 μl media to rows A-C of column 12.
14. Next add 6 μl of extract dilution to rows A-C of columns 1-10 in the plate. (Note: Each column number in the plate should correspond to the tube # from above.)
15. Add 6 μl of solvent to rows A-C of column 11.
16. Look at plate and tap it gently to be sure that extract is in the liquid in each well and not on the sidewall of it.
17. Incubate plate at 37° C. overnight w/CO₂ for 24 hours.
18. Put 500 μl from your original microfuge tube of cells (freshly-vortexed) into 10 mL of growth medium in a T-75 flask for a 1:2 split and leave at 37° C. until ready to split again.
19. Take this time to calculate the precise μg/mL of extract in each column, based on how much you measured out and how much volume you added to each column.
   Day Two
1. Aspirate off liquid in each well. Using multi-channel pipettor, wash each well once with 200 μl sterile PBS. Add 100 μl sterile media to each well.
2. Check cells under the microscope to be sure that they're still there and that they're not purple from internalized extract.
3. Remove 400 μl MTT reagent (which is stored in the door of the 4° C. in the BSL3 room) from the bottle to a microfuge tube. Add 10 μl MTT reagent per well using the regular pipettor, changing tips between each well and being careful not to contaminate the stock of MTT reagent. Incubate plate at 37° C. for 2 hours.
4. Using the multi-channel pipettor, add 100 μl detergent reagent (stored at room temp) per well. DO NOT SHAKE THE PLATE FROM HERE ON OUT. Cover plate with aluminum foil and leave plate at 37° C. until 3:00 p.m., at which time you should read the plate on the Tecan.
   Read Plate:
1. Using the Tecan plate reader, measure the absorbance of the wells at 560 nm. Use the program called “MTT” in XFluor4. Be sure that filter slide C is in the Tecan.
   Determine the average values from triplicate readings and subtract these average values from the average for the medium-only blank (column 12). Plot absorbance on the y-axis and μg/mL extract on the x-axis.

### EXAMPLE 9

Assay for Inhibition of Influenza A Infection by Elderberry Extractions

Day 1

1. Measure out extract on super-sensitive balance by window in WH 265. Start with at least 40 mg/mL. This would be 5 mg (or 0.005 g) per 125 μl of sterile PBS.

2. Vortex to dissolve. If it doesn't go into solution, add same amount of PBS. Repeat if necessary. If after this third try, it doesn't completely go into solution, spin down at 10-13,000 rpm for 30 sec in microcentrifuge. Remove supernatant and use instead. However, label and store insoluble fraction at −20° C.

3. Repeat steps 1 & 2 and combine the measured solubilized extract to prepare 250 μl of the extract solution.

4. Label 2 sterile microfuge tubes “Ab 1:1000” and “Ab 1:500”. Add 999 μl sterile PBS and 1 μl anti-influenza primary A antibody to the “Ab 1:1000” tube. Vortex. Add 998 μl PBS and 2 μl anti-influenza A primary antibody to the “Ab 1:500” tube. Vortex.

5. Dilute the virus:

a. Label 4 microfuge tubes “UV”, “−1”, “−2”, and “−3”. Add 990 μl PBS to the “UV” tube and 900 μl PBS to the others.
b. Add 10 μl of the virus on ice to the “UV” tube. Vortex. Change tip. Take 100 μl of that and add to the “−1” tube. Vortex. Continue, adding 100 μl from each tube to the next, vortexing and changing tip between each dilution.
   6. Dilute the extract:
a. Label 5 microfuge tubes “1:2”, “1:4”, “1:8”, “1:16”, and “1:32”. Add 125 μl PBS to each.
b. Vortex the extract solution. Add 125 μl of extract solution to the “1:2” tube. Vortex and change tip. Add 125 μl of “1:2” to “1:4”. Vortex and change tip. Add 125 μl of “1:4” to “1:8”. Repeat for remaining tubes, vortexing and changing tips between dilutions.
   7. Set up assay:
a. Label 7 microfuge tubes “undiluted”, “1:2”, “1:4”, “1:8”, “1:16”, “1:32”, and “PBS”.
b. Add 600 μl PBS to all but the “PBS” tube, which gets 1000 μl PBS.
c. Add 100 μl of “−3” virus dilution (FRESHLY VORTEXED!) to all 6 tubes—not to the “PBS” tube.
d. Vortex your “1:2” extract solution. Add 100 μl of “1:2” extract solution to your new “1:2” tube. Vortex.
e. Repeat step d for the “1:4” through “1:10” tubes, adding the extract dilutions to their respectively-labeled new tubes containing PBS and virus.
f. Add 100 μl of your undiluted extract solution (FRESHLY VORTEXED!) to the “undiluted” tube containing PBS and virus. Vortex.
g. Set up another tube with 100 μl of −3 virus and 700 μl of PBS and label it “−4 virus”. Vortex.
h. Immediately discard 300 μl from “Ab 1:1000” and “Ab 1:500” tubes and add 100 μl of the “−3” virus dilution (FRESHLY VORTEXED!) to each of the “Ab 1:1000” and “Ab 1:500” tubes. Vortex.
i. Set timer for 1 hour.
j. Turn off the light in the hood during this pre-incubation stage.
k. Label your plates with each triplicate column labeled “Undiluted extract”, “1:2”, “1:4”, “1:8”, “1:16”, “1:32”, “1:1000” and “1:500” for antibody controls, “−4 virus+PBS only” and “PBS only”.
l. About 50 minutes into the pre-incubation, wash the cells 3 times in PBS, leaving the wells empty for the next step.
m. AFTER THE HOUR of pre-incubation is over, vortex each tube just before you add 200 μl from it to each respectively-labeled well.
n. Incubate at room temp on Belly Dancer for 30 min., rotating 900 after 15 min and making agar overlay at this point, too.
   8. When you have about 15 minutes left in your infection, set up agar overlay:
a. Add DMEM bottle to water bath to warm it up.
b. Microwave 5% SeaPlaque stock for 1.5-2 minutes.

c. Mix the following in a sterile glass bottle that holds at least 100 mL:

AGAR OVERLAY

| | For 16-well plate: | | For 56-well plates: | |
|---|---|---|---|---|
| DMEM, warmed to 50° C. | 11.56 | mL | 57.8 | mL |
| Antibiotic-antimycotic | 150 | μl | 750 | μl |
| 7.5% BSA^{‡} | 0.576 | μl | 2.88 | mL |
| Glutamax | 150 | μl | 750 | μl |
| Trypsin (1 mg/mL)* | 14.4 | μl | 72 | μl |
| 5% Sea Plaque Agarose^{†} | 2.55 | mL | 12.75 | mL |
| | 15 | mL total vol. | 75 | mL total vol. |

^{‡}To make BSA, add 0.75 g BSA to 10 mL CaMg-PBS and filter-sterilize in the hood. Aliquot into 1.5-mL aliquots and store at −20° C.
*Trypsin is made up in 8.5 g/L NaCl—H₂O solution, filter sterilized in the hood, aliquotted into 1 mL aliquots, and stored at −20° C.
^{†}Add 5 g agarose to 100 mL H₂O and autoclave. Store at RT.

d. Remove inoculum and replace with 2 mL agar overlay per well. Leave plates right-side-up at 4° C. for about 20 minutes.
e. Remove plates from fridge and place right-side-up in 37° C. incubator for 27 hours post infection (after you added virus to your cells in step m).
   Day 2
   27 hours after infecting, add 0.5-1 mL Formafresh to each well. Leave plates at 4° C. overnight.
   Day 3
1. Aspirate off Formafresh.
2. Remove agar plugs with spatula.
3. Add 0.5 mL of 70% EtOH and incubate at room temp. for at least 20 min. Meanwhile, make up primary antibody at 1:1000 in Blotto in a 50 cc conical tube upstairs, vortexing to mix ingredients:
   15.5 mL PBS
   0.775 g dry milk
   15.5 μl Tween 20
   15.5 μl anti-influenza A antibody (kept at 4° C.)
4. Aspirate off EtOH. Rinse once with PBS.
5. Add 500 μl freshly-vortexed primary antibody in Blotto to each well. Rock upstairs at 4° C. on Belly Dancer overnight.
   Day 4
   1. Upstairs, mix up secondary antibody 1:500 in Blotto. (So, make up Blotto as before, only add 62 μl of secondary antibody (that has been frozen in glycerol, aliquotted, and stored at −20° C.) instead of primary antibody.)
   2. Take plates downstairs and aspirate off primary antibody.
   3. Wash once in PBS.
   4. Add 500 μl per well of your secondary antibody in Blotto and incubate for 5 hours at room temp on Belly Dancer.
   5. Aspirate off secondary antibody. Rinse once with PBS.
   6. Add 6 drops per well of Dakko substrate (kept in door of 4° C. downstairs in P3)
   7. Immediately put on Belly Dancer and incubate room temp. for 10-15 min. or until you see foci.
   8. Aspirate off substrate and wash once with PBS. Store in PBS.
   9. Photograph on light box and count foci.

### EXAMPLE 10

HIV Inhibition Protocol to Assay Elderberry Extract Activity

Pseudotyped HIV-1 Production

Pseudotyped HIV-1 virions were produced by co-transfecting 293T cells in T75 cell culture flasks with 6 μg of pSG₃^{Eenv}, a plasmid containing an envelope-deficient copy of the genome of HIV-1 strain SG3, and 2 μg of the envelope clone ZM53M.PB12, coding for the envelope of a subtype C HIV-1 strain from Zambia. Effectene Transfection Reagent (Qiagen, Valencia, Calif.) was used to transfect the cells. After 18 h the culture and medium with Effectene Transfection Reagent was replaced. Supernatants were collected 48 h post-transfection, clarified by low-speed centrifugation, aliquoted, and frozen at −18° C. The titers of he viral stocks were determined by infecting GHOST cells, seeded on a 96-wells plate, for 2 h at 37° C. with serial ten-fold dilutions. After 2 h incubation the medium with the virus was replaced with fresh Dulbecco modified Eagle medium containing 10% fetal bovine serum and incubated for 48 h at 37° C. The plate was scanned and foci counted using a Typhoon phosphorimager with ImageQuant software (Amersham Bioscience, Piscataway, N.J.).

Elderberries Extract Preparation (F4 Fraction) and Infection Inhibition Assays

Elderberries extract (F4) was prepared by re-suspending 40 mg of lyophilized elderberries extract in 1 mL of PBS (pH 7.2) and bringing it completely into solution by adjusting its pH to 7.0 with 40 μL of NaOH 0.625M. To assay F4 antiviral activity against HIV-1, 5×10⁴ GHOST cells were plated in each well of a 96-well tissue culture plate. The following day, ˜1,000 p.f.u. of psuedotyped virus were added to each well in the presence or absence of 6.55, 3.28, 1.64, 0.82, 0.41, and 0.20 μg of F4/mL. After 2 h of incubation at 37° C., the virus containing medium was removed and 200 μL of Dulbecco modified Eagle medium containing 10% fetal bovine serum was added per well and 37° C., incubation was continued for 48 h. Subsequently, the plate was scanned and foci counted using a Typhoon phosphorimager with ImageQuant software (Amersham Bioscience).

HIV-1 Subtype C Inhibition Assay

Inhibition assay for chimeric HIV-1 SG3 (genome) subtype C (envelope). This specific envelope protein comes from envelope clone ZM135M.PB12, GeneBank accession number AY423984, originated in Zambia, mode of transmission Female to Male, provided by Drs. E. Hunter and C. Derdeyn. The bright, white spots (see FIG. 9) are the foci on a slightly milky background. The background is caused by a slight fluorescence of the host cells and can not be further decreased. +, Positive infection control; F4, elderberry extract fraction F4; T titration of the virus used in the assay.

### INCORPORATION BY REFERENCE

All of the U.S. patents and U.S. patent application publications cited herein are hereby incorporated by reference.

### EQUIVALENTS

Those skilled in the art will recognize, or be able to ascertain using no more than routine experimentation, many equivalents to the specific embodiments of the invention described herein. Such equivalents are intended to be encompassed by the following claims.

We claim:

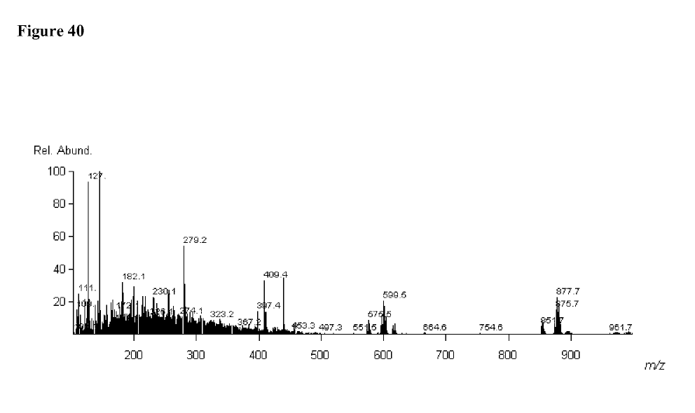

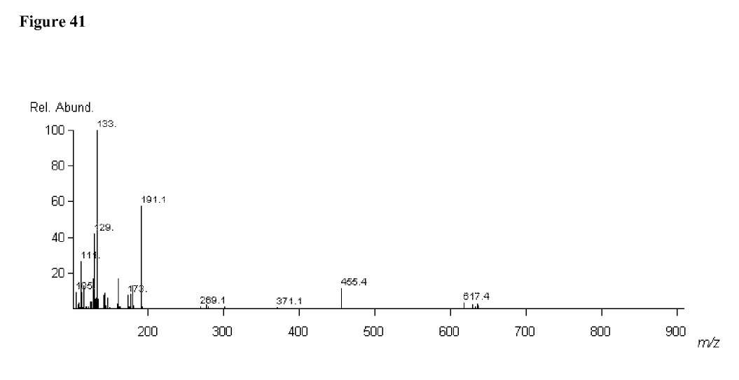

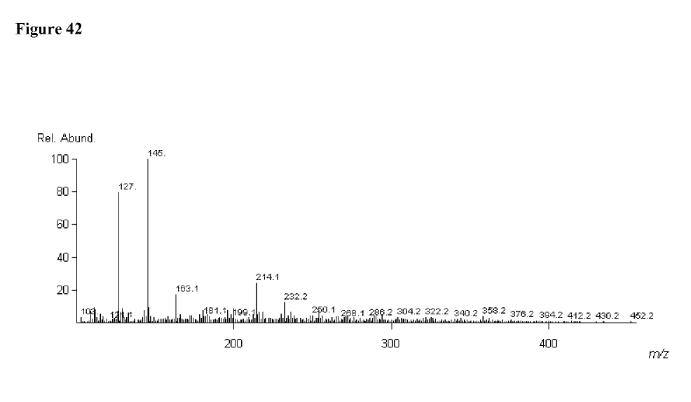

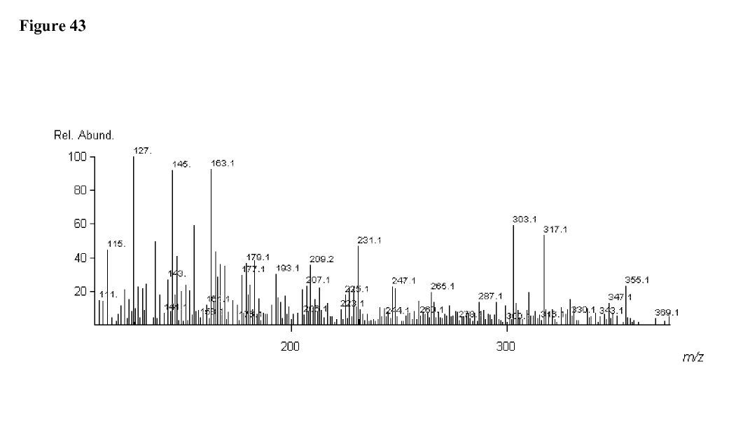

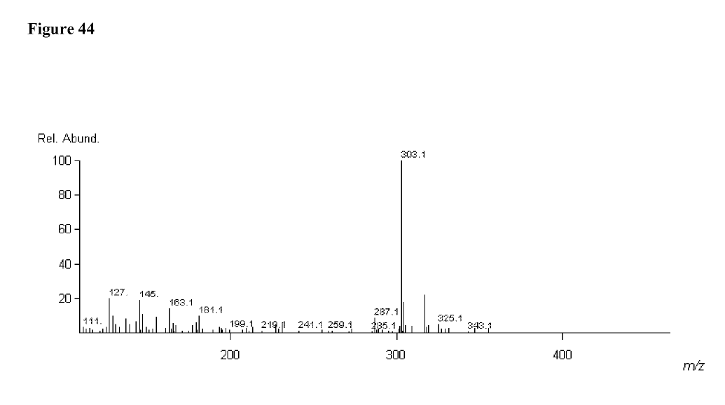

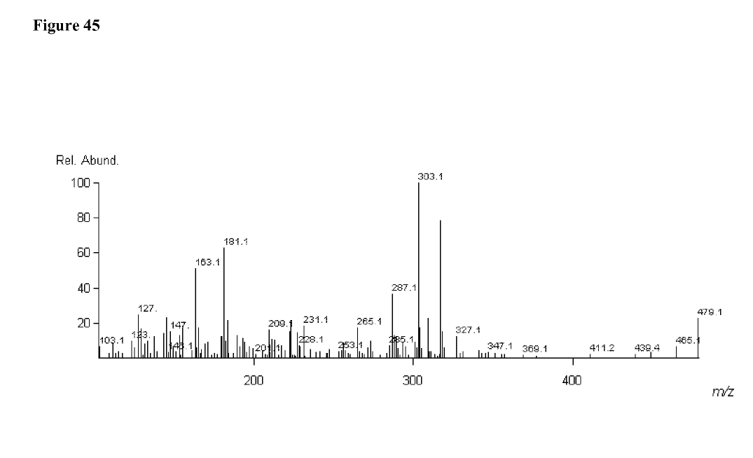

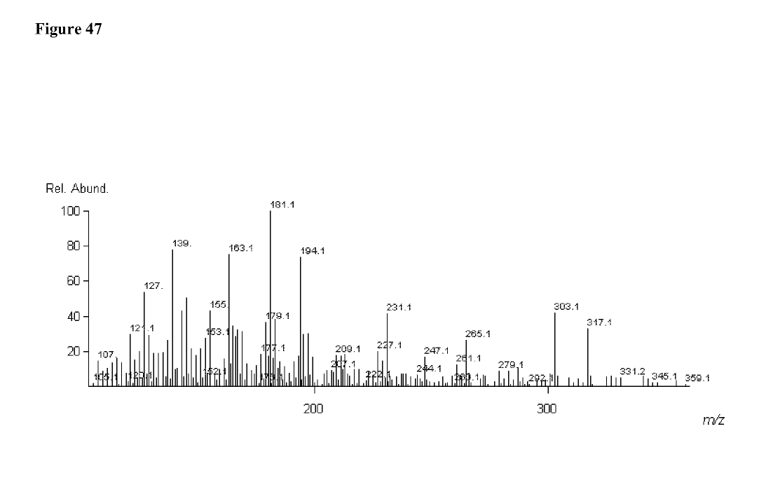

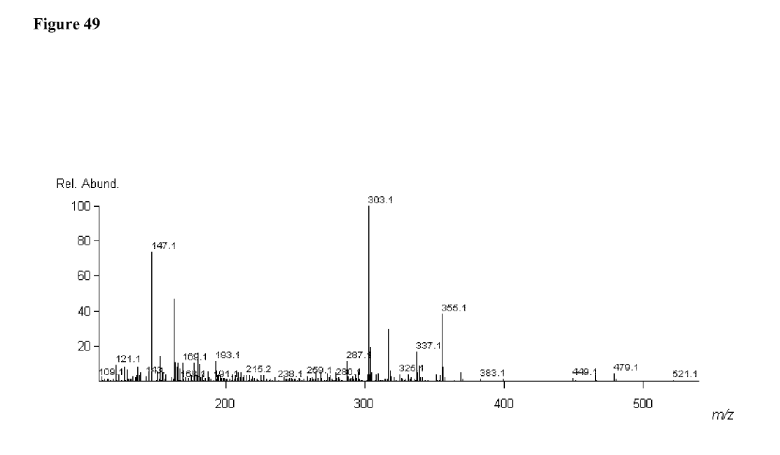

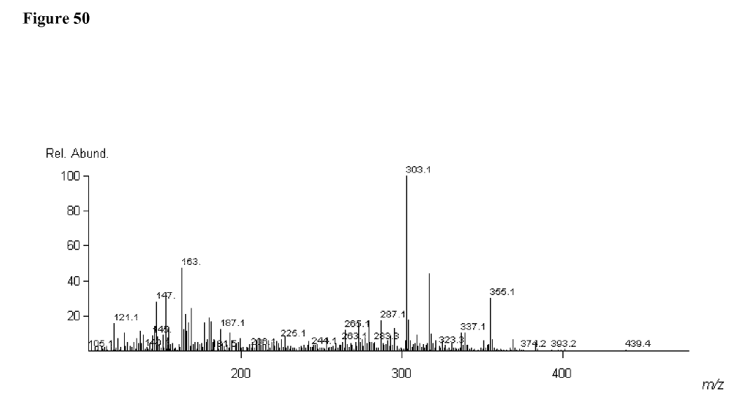

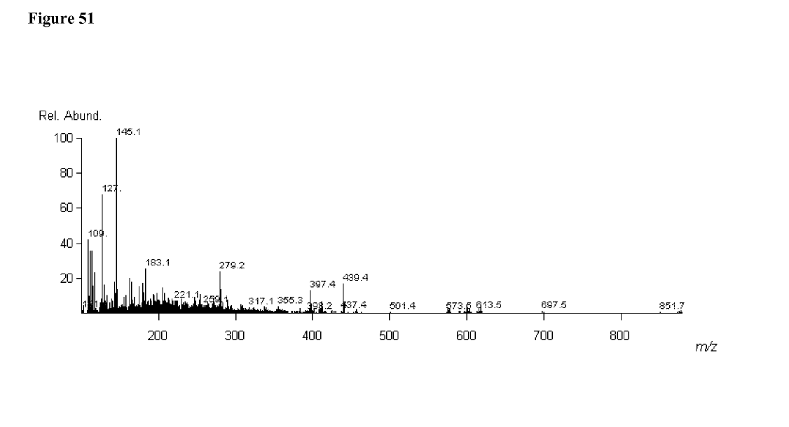

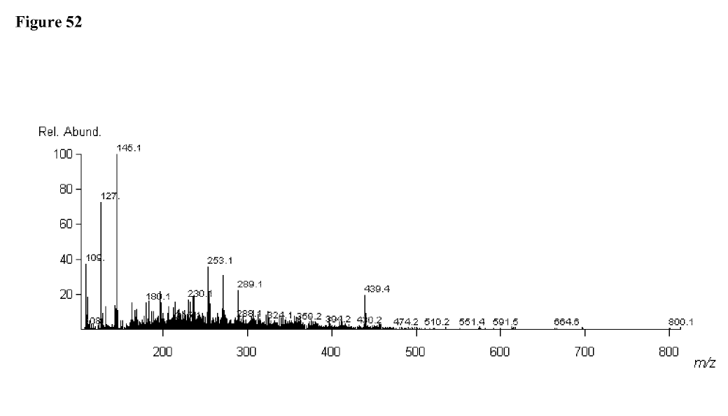

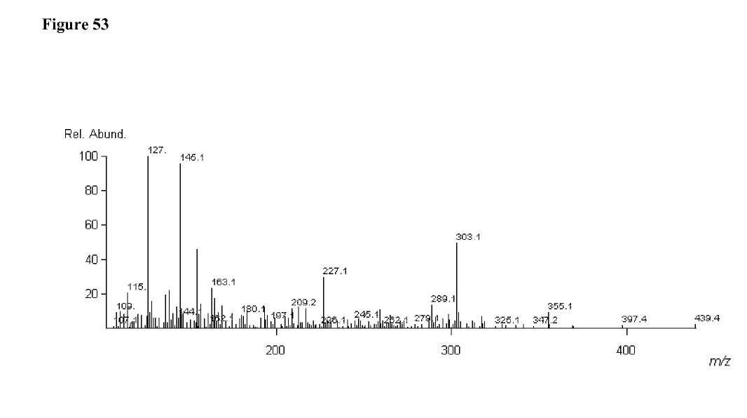

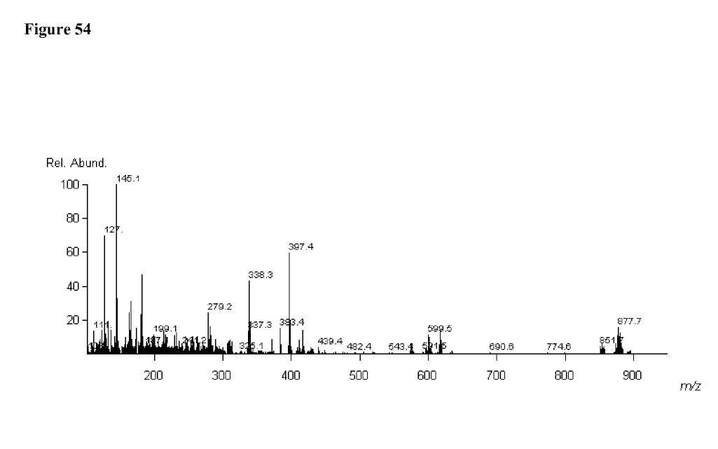

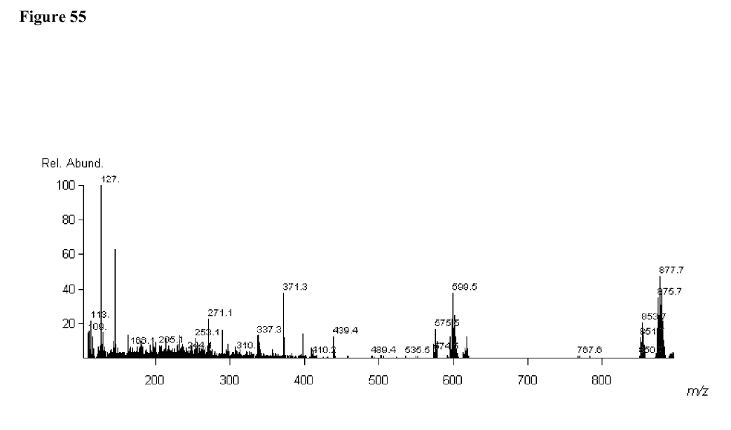

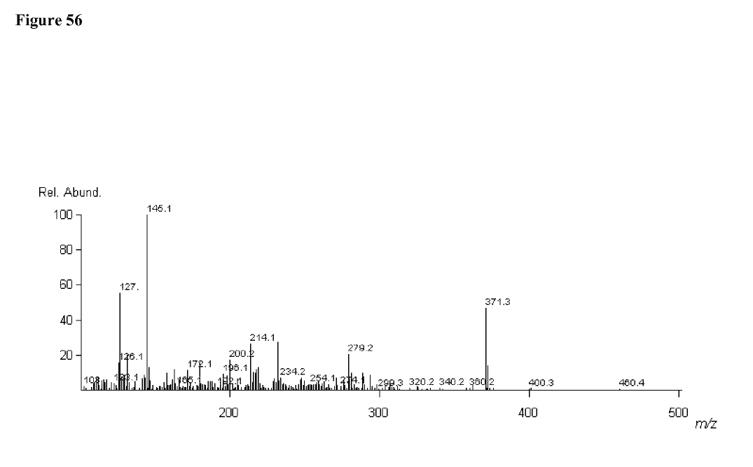

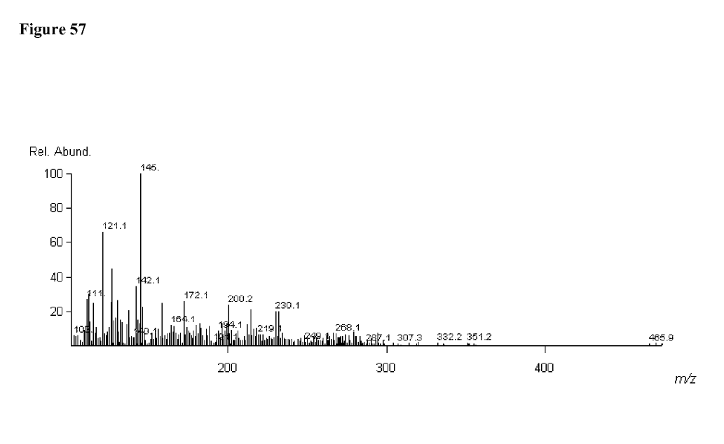

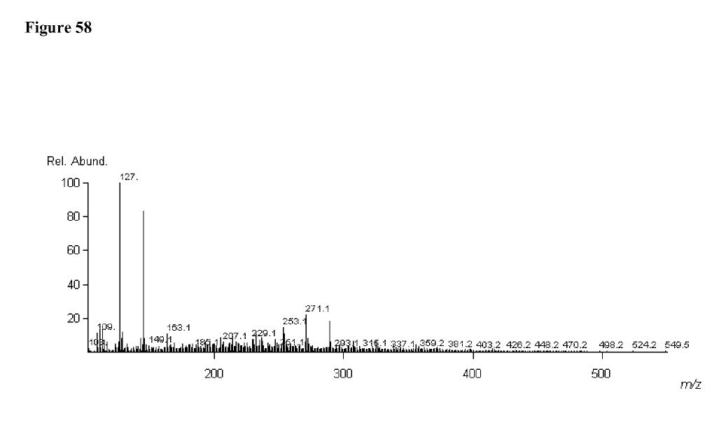

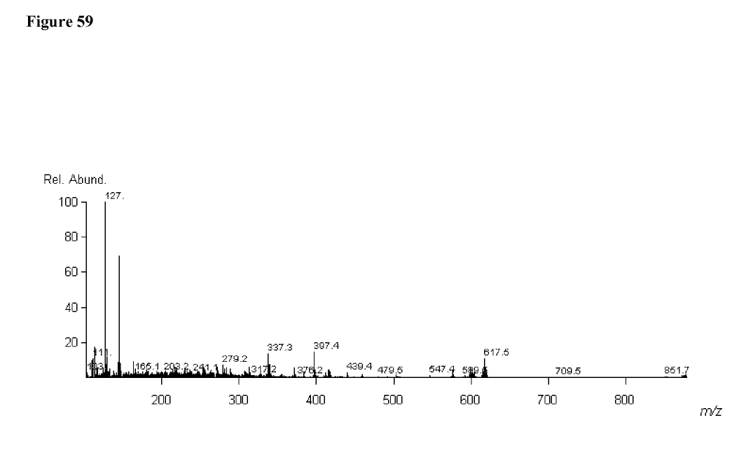

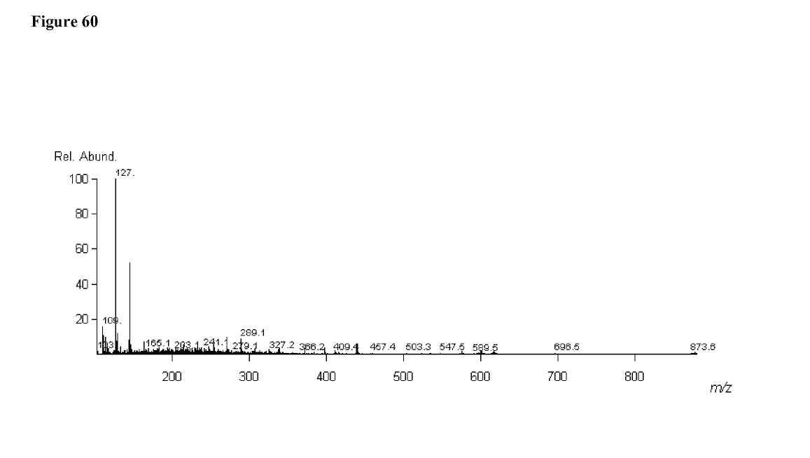

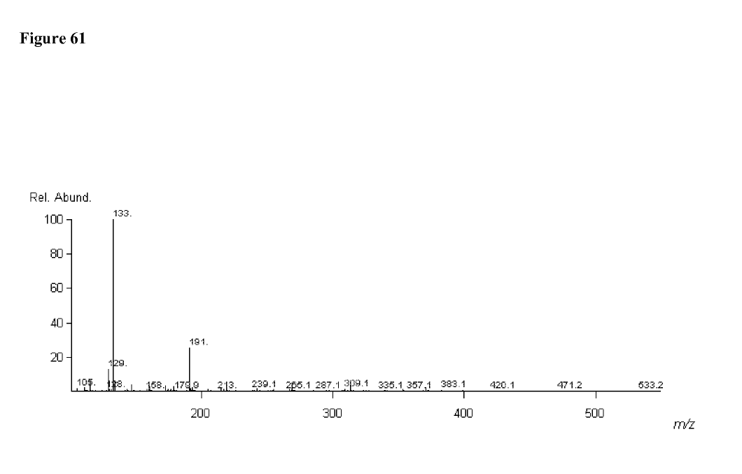

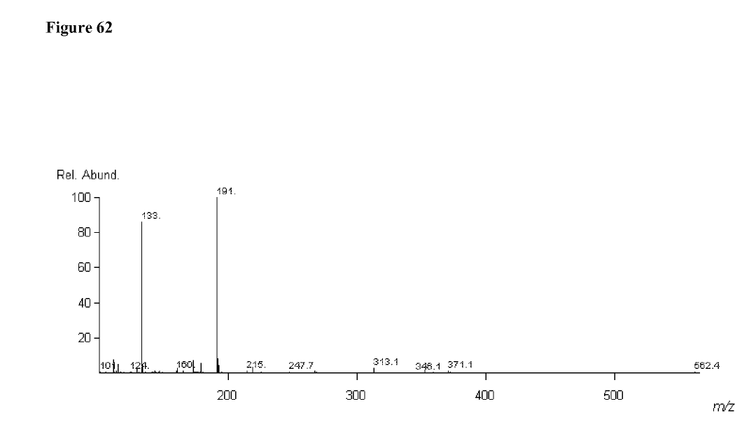

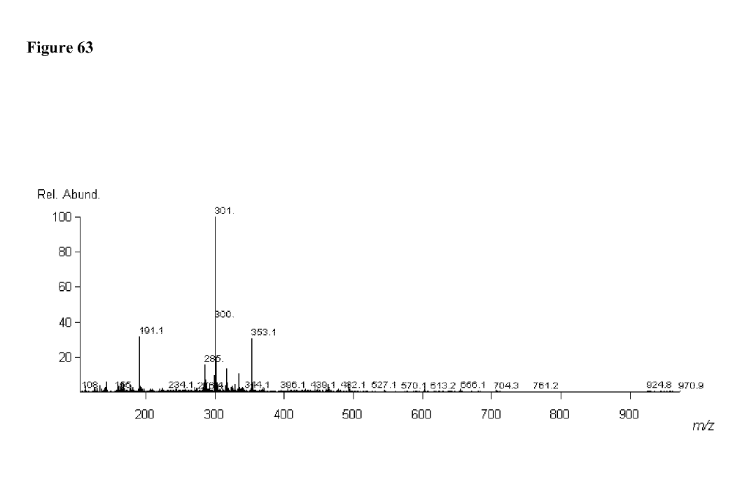

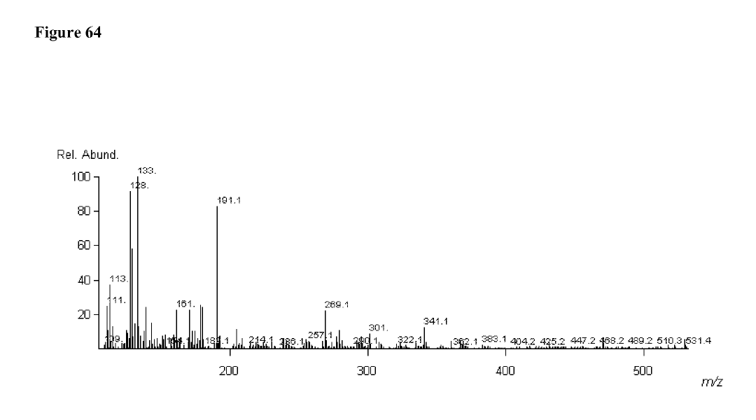

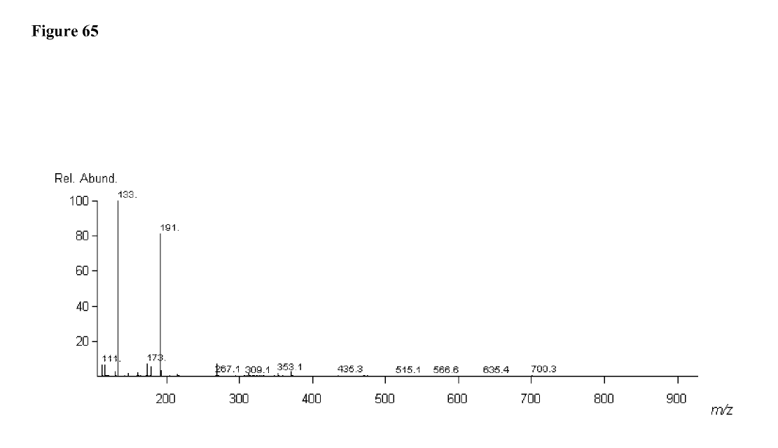

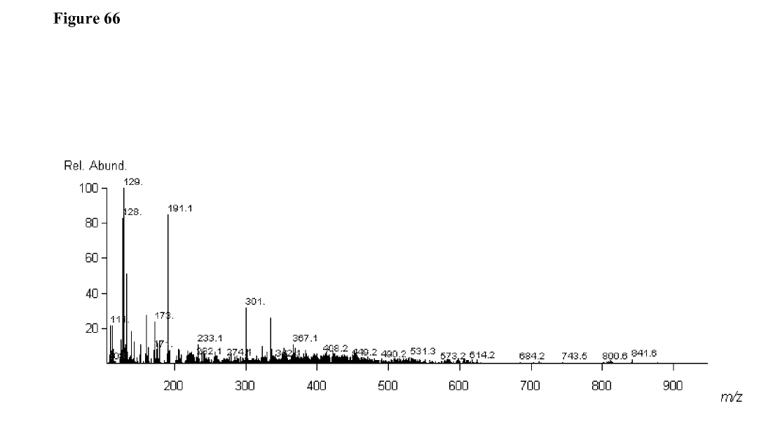

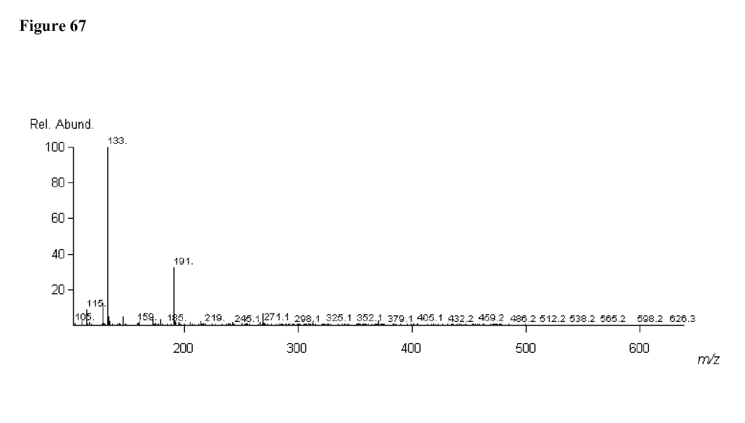

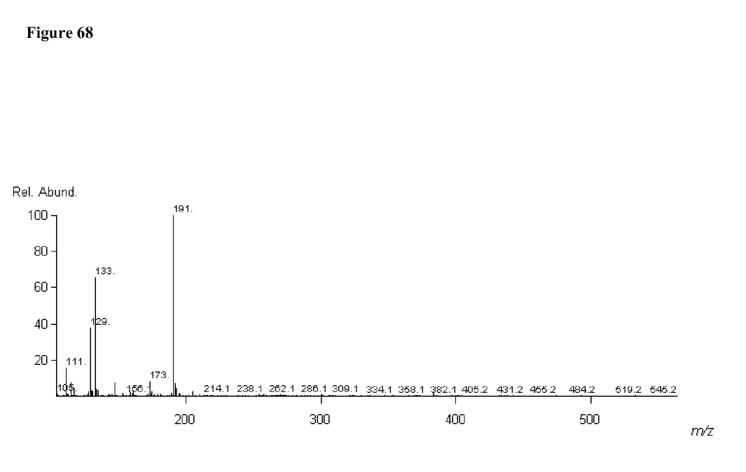

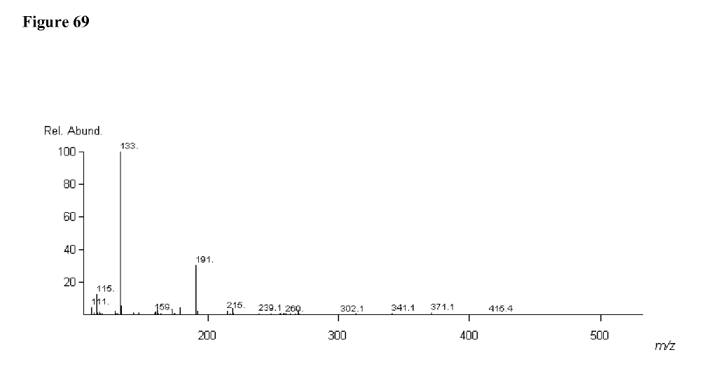

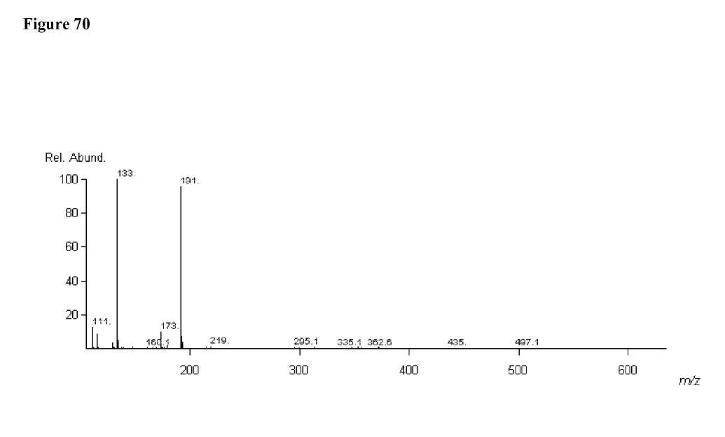

1. An elder species extract comprising a fraction having a Direct Analysis in Real Time (DART) mass spectrometry chromatogram of any of FIGS. 36 to 70.

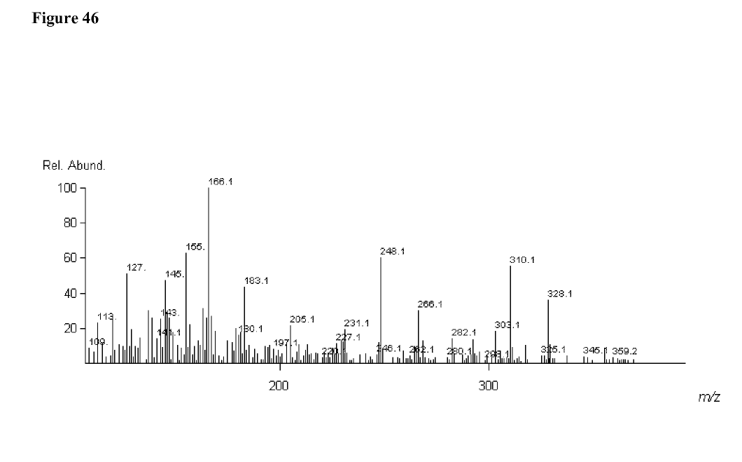

2. The elder species extract of claim 1, wherein the fraction has a DART mass spectrometry chromatogram of any of FIGS. 46 to 50.

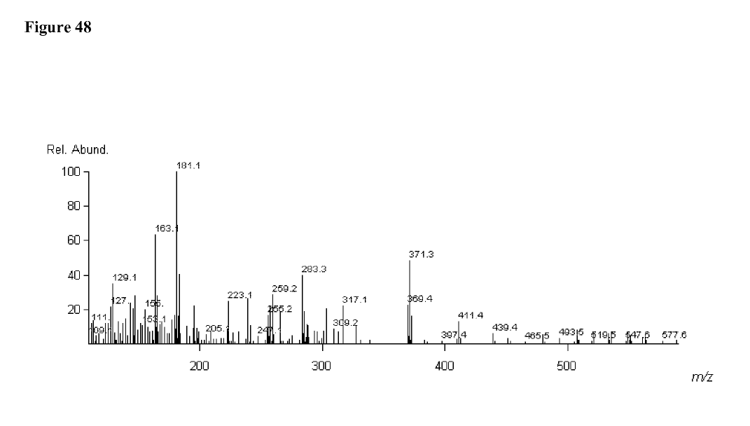

3. The elder species extract of claim 1, wherein the fraction has a DART mass spectrometry chromatogram of FIG. 48.

4. An elder species extract comprising a fraction having an IC₅₀ of 150 to 1500 μg/mL as measured in a H1N1 influenza inhibition assay.

5. The elder species extract of claim 4, wherein the fraction has an IC₅₀ of 150 to 750 μg/mL as measured in a H1N1 influenza inhibition assay.

6. The elder species extract of claim 4, wherein the fraction has an IC₅₀ of 150 to 300 μg/mL.

7. The elder species extract of claim 4, wherein the fraction has an IC₅₀ of at least about 195 μg/mL.

8. The elder species extract of claim 1 or 4, wherein the fraction comprises an anthocyanin; flavonoid; C16 or C18 saturated or unsaturated fatty acid, alcohol, or ester; and/or a polysaccharide.

9. The elder species extract of claim 8, wherein the anthocyanin is selected from the group consisting of cyanidin-3-glucoside and cyanidin-3-sambucioside.

10. The elder species extract of claim 8, wherein the amount of anthocyanins is greater than 10% by weight.

11. The elder species extract of claim 8, wherein the flavonoid is rutin.

12. The elder species extract of claim 8, wherein the C16 or C18 saturated or unsaturated fatty acid, alcohol, or ester is selected from the group consisting of hexadecanol, hexadecanoic acid, hexadecanoic acid methyl ester, hexadecanoic acid ethyl ester, hexadecanoic acid butyl ester, octadecanoic acid, octadecanoic acid ethyl ester, octadecanoic acid butyl ester, 9-octadecen-1-ol, 9,12-octadecanienoic acid, and combinations thereof.

13. The elder species extract of claim 8, wherein the amount of the C16 or C18 saturated or unsaturated fatty acid, alcohol, or ester is at least about 2% by weight.

14. The elder species extract of claim 8, wherein the polsaccharide is selected from the group consisting of dextran, glucose, arabinose, galactose, rhamnose, xylose, uronic acid, and combinations thereof.

15. The elder species extract of claim 8, wherein the amount of polysaccharide is at least about 10% by weight.

16. The elder species extract of claim 8 comprising an anthocyanin; C16 or C18 saturated or unsaturated fatty acid, alcohol, or ester; and a polysaccharide.

17. The elder species extract of claim 16, wherein the anthocyanin is selected from the group consisting of cyanidin-3-glucoside and cyanidin-3-sambucioside.

18. The elder species extract of claim 16, wherein the amount of anthocyanin is greater than 10% by weight.

19. The elder species extract of claim 16, wherein the C16 or C18 saturated or unsaturated fatty acid, alcohol, or ester is selected from the group consisting of hexadecanol, hexadecanoic acid, hexadecanoic acid methyl ester, hexadecanoic acid ethyl ester, hexadecanoic acid butyl ester, octadecanoic acid, octadecanoic acid ethyl ester, octadecanoic acid butyl ester, 9-octadecen-1-ol, 9,12-octadecanienoic acid, and combinations thereof.

20. The elder species extract of claim 16, wherein the amount of the C16 or C18 saturated or unsaturated fatty acid, alcohol, or ester is at least about 2% by weight.

21. The elder species extract of claim 16, wherein the polysaccharide is selected from the group consisting of dextran, glucose, arabinose, galactose, rhamnose, xylose, uronic acid, and combinations thereof.

22. The elder species extract of claim 16, wherein the amount of polysaccharide is at least about 10% by weight.

23. Food or medicament comprising the elder species extract of claim 1 or 4.

24. A method of treating a subject infected with a virus comprising administering to the subject in need thereof an effective amount of the elder species extract of claim 1 or 4.

25. The method of claim 24, wherein the virus is an envelope virus.

26. The method of claim 25, wherein the envelope virus is a flavie virus.

27. The method of claim 24, wherein the virus is a non-envelope virus.

28. The method of claim 24, wherein the virus is selected from the group consisting of influenza viruses, human flu viruses A and B, avian flu viruses, H1N1, H5N1, human immunodeficiency virus (HIV), SARs, herpes simplex viruses (HSV), flaviviruses, dengue, yellow fever, West Nile, and encephalitis viruses.

29. The method of claim 24, wherein the virus is selected from the group consisting of Norwalk virus, hepatitis A, polio, andoviruses and rhinoviruses.

30. The method of claim 24, wherein the subject is a primate, aviary, bovine, ovine, equine, porcine, rodent, feline, or canine.

31. The method of claim 24, wherein the subject is a human.

32. A method of inhibiting virus infection of cells comprising contacting the cells with the elder species extract of claim 1 or 4.

33. The method of claim 32, wherein the virus is an envelope virus.

34. The method of claim 33, wherein the envelope virus is a flavie virus.

35. The method of claim 32, wherein the virus is a non-envelope virus.

36. The method of claim 32, wherein the virus is selected from the group consisting of influenza viruses, human flu viruses A and B, avian flu viruses, H1N1, H5N1, human immunodeficiency virus (HIV), SARs, herpes simplex viruses (HSV), flaviviruses, dengue, yellow fever, West Nile, and encephalitis viruses.

37. The method of claim 32, wherein the virus is selected from the group consisting of Norwalk virus, hepatitis A, polio, andoviruses and rhinoviruses.

38. A method of preparing an elder species extract having at least one predetermined characteristic comprising: sequentially extracting an elder species plant material to yield an essential oil fraction, a polyphenolic fraction and a polysaccharide fraction by
a) extracting an elder species plant material by supercritical carbon dioxide extraction to yield the essential oil fraction and a first residue;
b) extracting either an elder species plant material or the first residue from step a) with water at about 40° C. to about 70° C. or with a hydro-alcoholic extraction to yield the polyphenolic fraction and a second residue; and
c) extracting the second residue from step b) by water at about 70° C. to about 90° C. extraction to yield the polysaccharide fraction.

39. The method of claim 38, wherein step a) comprises:
1) loading in an extraction vessel ground elder species plant material;
2) adding carbon dioxide under supercritical conditions;
3) contacting the elder species plant material and the carbon dioxide for a time; and
4) collecting an essential oil fraction in a collection vessel.

40. The method of claim 39, further comprising the step of altering the essential oil chemical constituent compound ratios by fractionating the essential oil extraction with a supercritical carbon dioxide fractional separation system.

41. The method of claim 38, wherein step b) comprises:
1) contacting ground elder species plant material or the residue from step a) with water at about 40° C. to about 70° C. or a hydro-alcoholic solution for a time sufficient to extract polyphenolic chemical constituents;
2) passing the water or hydro-alcoholic solution of extracted polyphenolic chemical constituents from step a) through an affinity adsorbent resin column wherein the polyphenolic acids including the anthocyanidins, are adsorbed; and
3) eluting the purified polyphenolic chemical constituent fraction(s) from the affinity adsorbent resin.

42. The method of claim 38, wherein the method for polysaccharide fraction extraction comprises:
1) contacting the second residue from step b) with water at about 70° C. to about 90° C. for a time sufficient to extract polysaccharides; and
2) precipitating the polysaccharides from the water solution by ethanol precipitation.

43. An elder species extract prepared by the method of any of claims 38 to 42.

44. An elder species extract comprising pyrogallol, methyl cinnamic acid at 15 to 25% by weight of the pyrogallol, cinnamide at 1 to 4% by weight of the pyrogallol, 2-methoxyphenol at 5 to 10% by weight of the pyrogallol, benzaldehyde at 1 to 2% by weight of the pyrogallol, cinnamaldehyde at 5 to 10% by weight of the pyrogallol, and cinnamyl acetate at 5 to 15% by weight of the pyrogallol.

45. An elder species extract comprising rutin, ferulic acid at 20 to 30% by weight of the rutin, cinnamic acid at 25 to 35% by weight of the rutin, shikimic acid at 15 to 25% by weight of the rutin, and phenyllacetic acid at 55 to 65% by weight of the rutin.

46. An elder species extract comprising rutin, taxifolin at 1 to 10% by weight of the rutin, ferulic acid at 1 to 5% by weight of the rutin, cinnamic acid at 1 to 5% by weight of the rutin, shikimic acid at 0.5 to 5% by weight of the rutin, phenyllacetic acid at 1 to 5% by weight of the rutin, cyanidin at 5 to 15% by weight of the rutin, and petunidin at 15 to 25% by weight of the rutin.

47. An elder species extract comprising rutin, cyanidin at 30 to 40% by weight of the rutin, petunidin at 75 to 85% by weight of the rutin, vanillic acid at 5 to 10% by weight of the rutin, ferulic acid at 1 to 5% by weight of the rutin, and cinnamic acid at 1 to 10% by weight of the rutin.

48. An elder species extract comprising p-coumaric acid/phenylpyruvic acid, rutin at 65 to 75% by weight of the p-coumaric acid/phenylpyruvic acid, vanillic acid at 65 to 75% by weight of the p-coumaric acid/phenylpyruvic acid, ferulic acid at 35 to 45% by weight of the p-coumaric acid/phenylpyruvic acid, cinnamic acid at 65 to 75% by weight of the p-coumaric acid/phenylpyruvic acid, and shikimic acid at 45 to 55% by weight of the p-coumaric acid/phenylpyruvic acid.

49. An elder species extract comprising rutin, hesperidin at 20 to 30% by weight of the rutin, vanillic acid at 70 to 80% by weight of the rutin, and cinnamic acid at 40 to 50% by weight of the rutin.

50. An elder species extract comprising petunidin, rutin at 85 to 95% by weight of the petunidin, vanillic acid at 55 to 65% by weight of the petunidin, and cinnamic acid at 30 to 40% by weight of the petunidin.

51. An elder species extract comprising rutin, cyanidin at 5 to 15% by weight of the rutin, taxifolin at 1 to 10% by weight of the rutin, caffeic acid at 5 to 15% by weight of the rutin, ferulic acid at 1 to 10% by weight of the rutin, shikimic acid at 1 to 10% by weight of the rutin, petunidin at 25 to 35% by weight of the rutin, and eriodictyol or fustin at 1 to 5% by weight of the rutin.

52. An elder species extract comprising rutin, cyanidin at 10 to 20% by weight of the rutin, eriodictyol or fustin at 1 to 5% by weight of the rutin, naringenin at 10 to 20% by weight of the rutin, and taxifolin at 1 to 10% by weight of the rutin.
